(19) United States
(12) Patent Application Publication
(10) Pub. No.: US 2026/0176634 A1
(43) Pub. Date: Jun. 25, 2026

(54) COMPOSITIONS AND METHODS FOR DECREASING TAU EXPRESSION

(71) Applicant: Novartis AG, Basel (CH)

(72) Inventors: Manuela POLYDORO OFENGEIM, Dedham, MA (US); Jan WEILER, Loerrach (DE)

(21) Appl. No.: 19/429,138

(22) Filed: Dec. 22, 2025

Related U.S. Application Data
(63) Continuation of application No. 17/813,843, filed on Jul. 20, 2022.
Application No. 17/813,843 is a continuation of application No. 15/383,452, filed on Dec. 19, 2016, now Pat. No. 11,473,083.
(60) Provisional application No. 62/270,165, filed on Dec. 21, 2015.

(51) Int. Cl. C12N 15/113; A61K 31/7125

(52) U.S. Cl. CPC C12N 15/113; A61K 31/7125; C12N 2310/11; C12N 2310/315; C12N 2310/321; C12N 2310/322; C12N 2310/3341; C12N 2310/341; C12N 2310/346; C12N 2320/30

(57) ABSTRACT

Provided herein are compositions and methods for decreasing tau mRNA and protein expression. These compositions and methods are useful in treating tau-related diseases and disorders.

### CROSS-REFERENCE TO RELATED APPLICATIONS

This application is a continuation of U.S. application Ser. No. 17/813,843 filed on Jul. 22, 2022, which is a continuation of U.S. application Ser. No. 15/383,452 filed on Dec. 19, 2016, which claims the benefit of U.S. Provisional Application No. 62/270,165, filed Dec. 21, 2015, which are incorporated by reference herein in their entirety.

### REFERENCE TO AN ELECTRONIC SEQUENCE LISTING

The contents of the electronic sequence listing (PAT057142-US-CNT02_ST26_SQL.xml; Size: 815,257 bytes; and Date of Creation: Dec. 22, 2025) are hereby incorporated by reference in their entirety.

### TECHNICAL FIELD

The present invention provides compositions and methods for decreasing tau mRNA and protein expression. These compositions and methods are useful for treating tau-associated diseases.

### BACKGROUND

Tau is a microtubule-associated protein that stabilizes microtubules and facilitates axonal transport. Tau protein interacts with tubulin to stabilize the microtubules and promote tubulin assembly into microtubules. The microtubule network is involved in many important cellular processes, including forming cytoskeleton and maintaining the structure and morphology of the cell, and providing platforms for intracellular transport of vesicles, organelles and macromolecules. Since binding of tau to microtubules stabilizes the microtubules, tau is a key mediator of these cellular processes.

At least six tau isoforms exist in human brain, ranging from 352-441 amino acid residues long. The tau isoforms are derived from a single gene MAPT (microtubule-associated protein tau) located on chromosome 17. MAPT transcript undergoes complex, regulated alternative splicing, giving rise to multiple mRNA species. Exons 2 and 3 of MAPT encode a 29- or 58-amino acid sequence respectively, and thus alternative splicing of exons 2 and/or 3 leads to inclusion of zero, one, or two copies of the N-terminal 29 amino acid acidic domain, which are referred to as 0N, 1N, or 2N tau, respectively. Exon 10 of MAPT encodes a microtubule-binding domain, thus inclusion of exon 10 leads to the presence of an additional microtubule-binding domain. Since there are three microtubule-binding domains elsewhere in tau, the tau isoforms that include exon 10 are referred to as “4R tau,” which means the tau protein with four repeats of microtubule-binding domain. The tau isoforms without exon 10 are referred to as “3R tau”, which means the tau protein with three repeats of microtubule-binding domain. The 4R tau isoforms presumably bind microtubules better than the 3R tau isoforms since they have one more microtubule-binding domain. The ratio of 3R tau to 4R tau is developmentally regulated, with fetal tissues expressing exclusively 3R tau and adult human tissues expressing approximately equal levels of 3R tau and 4R tau.

Tau is a phosphoprotein with approximately 85 potential phosphorylation sites (Ser, Thr, or Tyr) on the longest tau isoform (Pedersen and Sigurdsson, Trends in Molecular Medicine 2015, 21 (6): 394). Phosphorylation has been reported on approximately half of these sites in normal tau proteins. Tau is dynamically phosphorylated and dephosphorylated during the cell cycle. Tau can only associate with microtubules in its dephosphorylated form, and thus phosphorylation of tau acts as a direct microtubule association-dissociation switch within the neuron. Under pathological conditions, tau protein becomes hyperphosphorylated, resulting in a loss of tubulin binding and destabilization of microtubules, followed by the aggregation and deposition of tau in pathogenic neurofibrillary tangles. Protease cleavage fragments of tau (Asp13, Glu391, and Asp421) have also been identified in neurofibrillary tangles.

### SUMMARY OF THE INVENTION

Provided herein are antisense oligonucleotides targeting human microtubule-associated protein tau (MAPT), compositions comprising the antisense oligonucleotides, and methods for decreasing tau mRNA and protein expression using these antisense oligonucleotides. The compositions and methods provided herein are useful in treating tau-associated diseases.

In one aspect, provided herein are oligonucleotides comprising a nucleobase sequence that has at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any of the nucleobase sequences provided in Tables 2-17, wherein C in any of the nucleobase sequences is either cytosine or 5-methylcytosine, and wherein at least one nucleotide of the oligonucleotide has a 2′-modification. These oligonucleotides are antisense oligonucleotides targeting human MAPT. The 2′-modification can be selected from the group consisting of 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In some embodiments, the 2′-modification is 2′-O-methoxyethyl (2′-O-MOE). In some embodiments, each C in any of the nucleobase sequences is 5-methylcytosine.

In some embodiments, the antisense oligonucleotides provided herein are 12 to 30 nucleobases in length. For example, an antisense oligonucleotide targeting MAPT can comprise 12, 13, 14, 15, 16, 17, 18, 19, 20, 21, 22, 23, 24, 25, 26, 27, 28, 29, or 30 nucleobases. In some embodiments, the antisense oligonucleotides targeting MAPT are 12 to 25 nucleobases in length. For example, an antisense oligonucleotide targeting MAPT can comprise 12, 13, 14, 15, 16, 17, 18, 19, 20, 21, 22, 23, 24, or 25 nucleobases. In some embodiments, the antisense oligonucleotides targeting MAPT are 15 to 20 nucleobases in length. For example, an antisense oligonucleotide targeting MAPT can comprise 15, 16, 17, 18, 19, or 20 nucleobases.

In some embodiments, the antisense oligonucleotides targeting MAPT provided herein are steric blockers. Such antisense oligonucleotides decrease tau mRNA and/or protein expression independent of RNAse H. The internucleoside linkage of steric blockers can be either phosphodiester or phosphorothioate linkages. In some embodiments, the antisense oligonucleotides targeting MAPT provided herein are steric blockers comprising a nucleobase sequence that has at least 70% (e.g., 70%, 75%, 80%, 81%, 82%, 83%, 84%, 85%, 86%, 87%, 88%, 89%, 90%9, 91%, 92%, 93%, 94%, 95%, 96%, 97%, 98%, 99%, or 100%) sequence identity to any of the sequences provided in Tables 2-8, wherein C in any of the nucleobase sequences is either cytosine or 5-methylcytosine, and wherein each nucleotide of the oligonucleotide has a 2′-modification. In some embodiments, an antisense oligonucleotide targeting MAPT comprises a nucleobase sequence that has at least 80% sequence identity to any of the sequences provided in Tables 2-8. In some embodiments, an antisense oligonucleotide targeting MAPT comprises a nucleobase sequence that has at least 90% sequence identity to any of the nucleobase sequences provided in Tables 2-8. In some embodiments, an antisense oligonucleotide targeting MAPT comprises any of the nucleobase sequences provided in Tables 2-8. In some embodiments, an antisense oligonucleotide targeting MAPT consists of any of the nucleobase sequences provided in Tables 2-8. In some embodiments, each C in any of the nucleobase sequences is 5-methylcytosine.

In some embodiments, an antisense oligonucleotide targeting MAPT has 2′-O-MOE modification at each nucleotide subunit.

In some embodiments, an antisense oligonucleotide targeting MAPT comprises a linker attached to the 3′ end of the oligonucleotide through a phosphate bridge, and the oligonucleotide has any of the following structures:

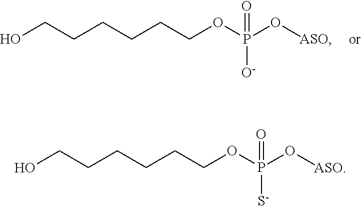

In some embodiments, the antisense oligonucleotides targeting MAPT provided herein are gapmers, which have a central gap segment of contiguous 2′-deoxyribonucleotides, positioned between two wing segments on the 5′ and 3′ ends (also called 5′ wing and 3′ wing respectively). Such antisense oligonucleotides decrease tau mRNA and/or protein expression by activating RNAse H. The internucleoside linkage of gapmers can be phosphorothioate or phosphodiester linkages. In some embodiments, the gapmers comprise a stretch of at least five (e.g., 5, 6, 7, 8, 9, 10, 11, 12) contiguous 2′-deoxyribonucleotides and the 5′ and 3′ wing segments comprise one or more 2′-modified nucleotides. In some embodiments, such an oligonucleotide comprises at least seven (e.g., 7, 8, 9, 10, 11, 12) contiguous 2′-deoxyribonucleotides. In some embodiments, such an oligonucleotide comprises ten contiguous 2′-deoxyribonucleotides. The 2′-modification can be selected from the group consisting of 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In some embodiments, the gapmers comprise 2′-O-MOE modified nucleotide in the 5′ wing and 3′ wing.

In some embodiments, the gapmers targeting tau are 5-10-5 gapmers that are 20 nucleosides in length, wherein the central gap segment comprises ten contiguous 2′-deoxynucleosides, flanked by 5′ wing and 3′ wing, each wing comprising five nucleosides each with a 2′-O-MOE modification.

In some embodiments, the antisense oligonucleotides targeting MAPT provided herein are gapmers comprising a nucleobase sequence that has at least 70% (e.g., 70%, 75%, 80%, 81%, 82%, 83%, 84%, 85%, 86%, 87%, 88%, 89%9, 90%, 91%, 92%, 93%, 94%, 95%, 96%, 97%, 98%, 99%, or 100%) sequence identity to any of the sequences provided in Tables 9-15 and 17, wherein C in any of the nucleobase sequences is either cytosine or 5-methylcytosine, and wherein at least one nucleotide of the oligonucleotide has a 2′-modification. In some embodiments, an antisense oligonucleotide targeting MAPT comprises a nucleobase sequence that has at least 80% sequence identity to any of the sequences provided in Tables 9-15 and 17. In some embodiments, an antisense oligonucleotide targeting MAPT comprises a nucleobase sequence that has at least 90% sequence identity to any of the nucleobase sequences provided in Tables 9-15 and 17. In some embodiments, an antisense oligonucleotide targeting MAPT comprises any of the nucleobase sequences provided in Tables 9-15 and 17. In some embodiments, an antisense oligonucleotide targeting MAPT consists of any of the nucleobase sequences provided in Tables 9-15 and 17. In some embodiments, the antisense oligonucleotides targeting MAPT provided herein are 5-10-5 gapmers that comprise any of the nucleobase sequences provided in any of Tables 9-15 and 17, wherein the first to fifth nucleotides each comprise a 2′-O-MOE modified nucleoside, wherein the sixth to fifteenth nucleotides each comprise a 2′-deoxynucleoside, and wherein the sixteenth to twentieth nucleotides each comprise a 2′-O-MOE modified nucleoside. In some embodiments, each C in any of the nucleobase sequences is 5-methylcytosine.

In some embodiments, the antisense oligonucleotide targeting MAPT comprises a nucleobase sequence selected from any one of SEQ ID NOs: 208, 284, 285, 313, 329, 335, 366, 384, 386, 405, 473, and 474. In some embodiments, the antisense oligonucleotide targeting MAPT comprises a nucleobase sequence that has at least 90% (e.g., 90%, 91%, 92%, 93%, 94%, 95%, 96%, 97%, 98%, 99%, or 100%) sequence identity to SEQ ID NO: 284. In some embodiments, the antisense oligonucleotide targeting MAPT comprises SEQ ID NO: 284. In some embodiments, the antisense oligonucleotide targeting MAPT comprises a nucleobase sequence that has at least 90% (e.g., 90%, 91%, 92%, 93%, 94%, 95%, 96%, 97%, 98%, 99%, or 100%) sequence identity to SEQ ID NO: 285 or 208. In some embodiments, the antisense oligonucleotide targeting MAPT comprises SEQ ID NO: 285 or 208.

In another aspect, provided herein are oligonucleotides comprising a nucleobase sequence that is complementary to at least 12 contiguous nucleobases of any one of SEQ ID NOs: 487-506, with 1, 2, or 3 mismatches, wherein at least one nucleotide of the oligonucleotide has a 2′-modification. These oligonucleotides are antisense oligonucleotides targeting MAPT. In some embodiments, such an oligonucleotide comprises a nucleobase sequence that is 100% complementary to at least 12 contiguous nucleobases of any one of SEQ ID NOs: 487-506. In some embodiments, such an oligonucleotide comprises one or more 5-methylcytosines. In some embodiments, such an oligonucleotide has a 2′-modification. The 2′-modification can be selected from the group consisting of 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In some embodiments, the 2′-modification is 2′-O-methoxyethyl (2′-O-MOE). In some embodiments, such an oligonucleotide comprises at least five (e.g., 5, 6, 7, 8, 9, 10, 11, 12) contiguous 2′-deoxyribonucleotides. In some embodiments, such an oligonucleotide comprises at least seven (e.g., 7, 8, 9, 10, 11, 12) contiguous 2′-deoxyribonucleotides. In some embodiments, such an oligonucleotide comprises ten contiguous 2′-deoxyribonucleotides.

In some embodiments, the antisense oligonucleotides targeting MAPT provided herein are capable of decreasing tau mRNA or protein expression level by at least 30% in vitro.

In some embodiments, the antisense oligonucleotides targeting MAPT provided herein are capable of decreasing tau mRNA or protein expression level by at least 30% in vivo.

In another aspect, provided herein are compositions comprising any of the antisense oligonucleotides described herein and a pharmaceutically acceptable carrier.

In a further aspect, provided herein are methods of decreasing tau expression level in a subject, e.g., a subject afflicted with or susceptible to a tau-associated disease, by administering to the subject a therapeutically effective amount of any of the antisense oligonucleotides described herein. In some embodiments, such methods can include administering a second agent to the subject. In some embodiments, the antisense oligonucleotide targeting MAPT can be administered to the subject through an intrathecal, intracranial, intranasal, oral, intravenous, or subcutaneous route. In some embodiments, the subject is a human.

Also provided are antisense oligonucleotides as described herein for use in treating a tau-associated disease in a subject in need thereof, e.g., a subject afflicted with or susceptible to a tau-associated disease. Use of the antisense oligonucleotides or pharmaceutical composition described herein to treat a tau-associated disease in a subject in need thereof is also included. The present disclosure also includes use of the antisense oligonucleotides described herein in the manufacture of a medicament for use in the treatment of a tau-associated disease in a subject in need thereof.

The tau-associated disease can be selected from Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, or tuberous sclerosis.

### BRIEF DESCRIPTION OF THE DRAWINGS

FIGS. 1 and 1A-1E show physical characterization of antisense oligonucleotide targeting MAPT. FIG. 1 shows the structure of antisense oligonucleotide (ASO) comprising SEQ ID NO: 284, with formula C230H321N72O120P19S19 and expected molecular weight of 7212.3 Da. FIG. 1A, FIG. 1A-1, FIG. 1A-2, FIG. 1A-3, and FIG. 1A-4 show enlarged view of FIG. 1. FIG. 1B shows the liquid chromatography-mass spectrometry (LC-MS) data of ASO comprising SEQ ID NO: 284, with a measured peak mass of 7214.3. FIG. 1C shows the deconvolution peak report of LC-MS for ASO comprising SEQ ID NO: 284. FIG. 1D shows the LC-MS data of ASO comprising SEQ ID NO: 285, with a measured peak mass of 7232.5. FIG. 1E shows the deconvolution peak report of LC-MS for ASO comprising SEQ ID NO: 285.

FIGS. 2A-2E show the expression level of human tau mRNA and protein in a representative hTau BAC transgenic mouse line before and after antisense oligonucleotide treatment. FIG. 2A is representative RT-PCR results showing that all six human tau transcripts were found in the forebrain of the hTau BAC transgenic mice (transgenic line 510, two month old female mice). Exons 2, 3 and 10 are alternatively spliced, resulting in six tau isoforms: 2−3−10−; 2+3−10−; 2+3+10−; 2−3−10+; 2+3−10+; 2+3+10+. 4R represents tau isoforms with exon 10, 3R represents tau isoforms without exon 10; 0N represents tau isoforms with neither exon 2 nor exon 3; 1N represents tau isoforms with either exon 2 or exon 3; 2N represents tau isoforms with both exon 2 and exon 3. FIG. 2B is a representative Western blot showing six tau protein isoforms ranging from 352-441 amino acids with molecular weights of 48-67 kD. They differ in (1) inclusion of zero, one or two inserts of a 29 amino acid N-terminal part (0N, 1N, or 2N), or (2) inclusion of three or four microtubule binding domain (3R or 4R). FIG. 2C is a representative immunohistochemistry image showing normal axonal distribution of human tau in the brain of hTau BAC transgenic mouse, as stained by a human tau specific antibody. FIG. 2D is a bar graph showing tau mRNA knockdown in the cortex of hTau BAC transgenic mouse 4 weeks after a single treatment of an antisense oligonucleotide comprising SEQ ID NO: 285. FIG. 2E is a representative Western blot showing tau protein knockdown in the hippocampus of hTau BAC transgenic mouse 4 weeks after a single treatment of an antisense oligonucleotide comprising SEQ ID NO: 285.

FIG. 3 is a set of in situ hybridization images showing wide brain distribution of the antisense oligonucleotide of SEQ ID NO: 285 in hTau BAC transgenic mice.

FIGS. 4A and 4B are dot plots showing dose-dependent inhibition of human tau mRNA (FIG. 4A) and protein (FIG. 4B) expression in hTau BAC transgenic mouse at 4 weeks or 12 weeks after a single ICV injection of 1, 10, 50, 200, or 400 ug of the antisense oligonucleotide of SEQ ID NO: 285.

FIGS. 5A and 5B are dot plots showing the time course of human tau mRNA (FIG. 5A) and protein (FIG. 5B) expression level in hTau BAC transgenic mouse after a single ICV injection of 200 ug of the antisense oligonucleotide of SEQ ID NO: 285.

### DETAILED DESCRIPTION

Provided herein are antisense oligonucleotides targeting microtubule-associated protein tau (MAPT), compositions comprising the antisense oligonucleotides, and methods for decreasing tau expression using these antisense oligonucleotides. The compositions and methods provided herein are useful in treating tau-associated diseases.

### Definitions

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely examples and that equivalents of such are known in the art.

The term “2′-modification” refers to substitution of H or OH at the 2′-position of the furanose ring of a nucleoside or nucleotide with another group.

As used herein, “2′-O-methoxyethyl,” “2′-MOE,” or “2′-OCH2CH2-OCH3” refers to an O-methoxyethyl modification of the 2′ position of a furanose ring. A 2′-O-methoxyethyl modified sugar is a modified sugar. A “2′-MOE nucleoside/nucleotide” or “2′-O-methoxyethyl nucleoside/nucleotide” refers to a nucleoside/nucleotide comprising a 2′-MOE modified sugar moiety.

A “5-methylcytosine” refers to a cytosine modified with a methyl group attached to the 5′ position.

The term “antisense oligonucleotide” as used herein refers to a single-stranded oligonucleotide having a nucleobase sequence that is complementary to a corresponding segment of a target nucleic acid, e.g., a target genomic sequence, pre-mRNA, or mRNA molecule. In some embodiments, an antisense oligonucleotide is 12 to 30 nucleobases in length.

The term “complementarity” or “complementary” refers to the capacity of base pairing between the nucleobases of a first nucleic acid strand and the nucleobases of a second nucleic acid strand, mediated by hydrogen binding (e.g., Watson-Crick, Hoogsteen or reversed Hoogsteen hydrogen bonding) between corresponding nucleobases. For example, in DNA, adenine (A) is complementary to thymine (T); and guanosine (G) is complementary to cytosine (C). For example, in RNA, adenine (A) is complementary to uracil (U); and guanosine (G) is complementary to cytosine (C). In certain embodiments, complementary nucleobase means a nucleobase of an antisense oligonucleotide that is capable of base pairing with a nucleobase of its target nucleic acid. For example, if a nucleobase at a certain position of an antisense oligonucleotide is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but are not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, the term “gapmer” refers to a chimeric antisense oligonucleotide comprising a central gap segment consisting of contiguous 2′-deoxyribonucleotides, which is capable of activating RNAse H, flanked by two wing segments on the 5′ and 3′ ends, each comprising one or more modified nucleotides, which confer increased resistance to nuclease degradation.

The term “hybridization” refers to the base pairing of complementary nucleic acid strands and formation of a duplex structure. Hybridization can occur between fully complementary nucleic acid strands or between “substantially complementary” nucleic acid strands that contain minor regions of mismatch. While not limited to a particular mechanism, the most common mechanism of pairing involves hydrogen bonding, which may be Watson-Crick, Hoogsteen or reversed Hoogsteen hydrogen bonding, between complementary nucleobases of the nucleic acid strands. For example, adenine and thymine are complementary nucleobases which pair through the formation of hydrogen bonds. Hybridization can occur under varying stringent circumstances. As used herein, hybridization refers to base pairing of complementary nucleic acid strands and formation of a duplex structure at least under a relatively low stringency condition, for example, hybridization in 2×SSC (0.3 M sodium chloride, 0.03 M sodium citrate), 0.1% SDS at 37° C., followed by washing in solution containing 4×SSC, 0.1% SDS can be used at 37° C., with a final wash in 1×SSC at 45° C.

The term “inhibiting” or “inhibition” refers to a reduction or blockade of the expression or activity of a target nucleic acid or protein and does not necessarily indicate a total elimination of expression or activity of the target.

The term “internucleoside linkage” refers to the chemical bond between nucleosides.

The term “knockdown” or “expression knockdown” refers to reduced mRNA or protein expression of a gene after treatment of a reagent, e.g., an antisense oligonucleotide. Expression knockdown can occur during transcription, mRNA splicing, or translation.

The term “mismatch” refers to the case where a nucleobase of a first nucleic acid strand is not complementary to the corresponding nucleobase of a second nucleic acid strand.

The term “nucleobase sequence” refers to the order of contiguous nucleobases independent of any sugar, linkage, and/or nucleobase modification.

The term “oligonucleotide” refers to a polymer of linked deoxyribonucleotides (DNA) and/or ribonucleotides (RNA), each of which is modified or unmodified. Unless specifically limited, the term encompasses nucleic acids containing known analogues of natural nucleotides that have similar binding properties as the natural nucleic acid, and nucleic acids having alternative internucleoside linkages other than phosphodiester linkages.

The term “phosphorothioate linkage” refers to a linkage between nucleosides where the phosphodiester bond is modified by replacing one of the non-bridging oxygen atoms with a sulfur atom.

The term “sense strand” refers to the coding strand, plus strand, or non-template strand of the DNA molecule which consists of a double stranded structure. The coding strand has the same sequence as the mRNA sequence except that Thymine (T) in DNA is replaced by Uracil (U) in RNA. An “antisense strand” refers to the non-coding strand or template strand of the DNA molecule, which acts as a template for synthesis of mRNA. Therefore, the sequence of antisense strand is complementary to the sequence of sense strand and mRNA (U in RNA in place of T).

As used herein, the term “steric blocker” refers to an antisense oligonucleotide that hybridizes with a target nucleic acid (e.g., a target genomic sequence, pre-mRNA, or mRNA molecule), and interferes with the transcription, splicing, and/or translation of the target nucleic acid without activating RNAse H.

As used herein, “targeting” or “targeted” refers to design and selection of an antisense oligonucleotide that can specifically hybridize to a target nucleic acid, e.g., a target genomic sequence, pre-mRNA, or mRNA molecule, or a fragment or variant thereof, and modulate the transcription, splicing, and/or translation of the target nucleic acid.

As used herein, “tau” (also known as “microtubule-associated protein tau”, MAPT, MSTD; PPND; DDPAC; MAPTL; MTBT1; MTBT2; FTDP-17; PPP1R103) refers to a microtubule-associated protein encoded by the gene MAPT. The human MAPT gene is mapped to chromosomal location 17q21.1, and the genomic sequence of human MAPT gene can be found in GenBank at NG_007398.1 (SEQ ID NO: 304). The MAPT intron and exon sequences and branch points can be determined based on Ensembl genomes database website using Transcript: MAPT-203 ENST00000344290. Due to complicated alternative splicing, eight tau isoforms are present in the human. The term “tau” is used to refer collectively to all isoforms of tau. The protein and mRNA sequences for the longest human tau isoform are:

| Homo sapiens microtubule-associated protein tau (MAPT), transcript | |
|---|---|
| variant 6, mRNA (NM_0011230663) | |
| (SEQ ID NO: 305) | |
| 1 | agacggccga gcggcagggc gctcgcgcgc gcccactagt ggccagagga gaaggctccc |
| 61 | gcggaggccg cgctgcccgc cccctcccct ggggaggctc gcgttcccgc tgctcgcgcc |
| 121 | tgcgccgccc gccggcctca ggaacgcgcc ctcttcgccg gcgcgcgccc tcgcagtcac |
| 181 | cgccacccac cagctccggc accaacagca gcgccgctgc caccgcccac cttctgccgc |
| 241 | cgccaccaca gccaccttct cctcctccgc tgtcctctcc cgtcctcgcc tctgtcgact |
| 301 | atcaggtgaa ctttgaacca ggatggctga gccccgccag gagttcgaag tgatggaaga |
| 361 | tcacgctggg acgtacgggt tgggggacag gaaagatcag gggggctaca ccatgcacca |
| 421 | agaccaagag ggtgacacgg acgctggcct gaaagaatct cccctgcaga cccccactga |
| 481 | ggacggatct gaggaaccgg gctctgaaac ctctgatgct aagagcactc caacagcgga |
| 541 | agatgtgaca gcacccttag tggatgaggg agctcccggc aagcaggctg ccgcgcagcc |
| 601 | ccacacggag atcccagaag gaaccacagc tgaagaagca ggcattggag acacccccag |
| 661 | cctggaagac gaagctgcta gtcacgtgac ccaagagcct gaaaatggta aggtggtcca |
| 721 | ggaaggcttc ctccgagagc caggcccccc aggtctgagc caccagctca tgtccggcat |
| 781 | gcctgggact cccctcctgc ctgagggccc cagagaggcc acacgccaac cttcggggac |
| 841 | aggacctgag gacacagagg gcggccgcca cgcccctgag ctgctcaagc accagcttct |
| 901 | aggagacctg caccaggagg ggccgccact gaagggggca gggggcaaag agaggccggg |
| 961 | gagcaaggag gagatggatg aagaccgcga cgtcgatgag tcctcccccc aagactcccc |
| 1021 | tccctccaag gcctccccag cccaagatgg gcggcctccc cagacagccg ccagagaagc |
| 1081 | caccagcatc ccaggcttcc cagcggaggg tgccatcccc ctccctgtgg atttcctctc |
| 1141 | caaagtttcc acagagatcc cagcctcaga gcccgacggg cccagtgtag ggcgggccaa |
| 1201 | agggcaggat gcccccctgg agttcacgtt tcacgtagaa atcacaccca acgtacagaa |
| 1261 | ggagcaggcg cactcggagg agcatttggg aagggctgca tttccagggg cccctggaga |
| 1321 | ggggccagag acccggggcc cctctttggg agaggacaca aaagaggctg accttccaga |
| 1381 | gccctctgaa aagcagcctg ctgctgctcc gcgggggaag cccgtcagcc gggtccctca |
| 1441 | actcaaagct cgcatgatca gtaaaagcaa agacgggact ggaagcgatg acaaaaaagc |
| 1501 | caagacatcc acacgttcct ctgctaaaac cttgaaaaat aggccttgcc ttagccccaa |
| 1561 | acaccccact cctggtaact cagaccctct gatccaaccc tccaaccctg ctatgtgccc |
| 1621 | agagccacct tcctctccta aatacgtctc ttctgtcact tcccgaactg gcagttctgg |
| 1681 | agcaaaggag atgaaactca agggggctga tggtaaaacg aagatcgcca caccgcgggg |
| 1741 | agcagcccct ccaggccaga agggccaggc caacgccacc aggattccag caaaaacccc |
| 1801 | gcccgctcca aagacaccac ccagctctgc gactaagcaa gtccagagaa gaccaccccc |
| 1861 | tgcagggccc agatctgaga gaggtgaacc tccaaaatca ggggatcgca gcggctacag |
| 1921 | cagccccggc tccccaggca ctcccggcag ccgctcccgc accccgtccc ttccaacccc |
| 1981 | acccacccgg gagcccaaga aggtggcagt ggtccgtact ccacccaagt cgccgtcttc |
| 2041 | cgccaagagc cgcctgcaga cagcccccgt gcccatgcca gacctgaaga atgtcaagtc |
| 2101 | caagatcggc tccactgaga acctgaagca ccagccggga ggcgggaagg tgcagataat |
| 2161 | taataagaag ctggatctta gcaacgtcca gtccaagtgt ggctcaaagg ataatatcaa |
| 2221 | acacgtcccg ggaggcggca gtgtgcaaat agtctacaaa ccagttgacc tgagcaaggt |
| 2281 | gacctccaag tgtggctcat taggcaacat ccatcataaa ccaggaggtg gccaggtgga |
| 2341 | agtaaaatct gagaagctta acttcaagga cagagtccag tcgaagattg ggtccctgga |
| 2401 | caatatcacc cacgtccctg gcggaggaaa taaaaagatt gaaacccaca agctgacctt |
| 2461 | ccgcgagaac gccaaagcca agacagacca cggggcggag atcgtataca agtcgccagt |
| 2521 | ggtgtctggg gacacgtctc cacggcatct cagcaatgtc tcctccaccg gcagcatcga |
| 2581 | catggtagac tcgccccagc tcgccacgct agctgacgag gtgtctgcct ccctggccaa |
| 2641 | gcagggtttg tgatcaggcc cctggggcgg tcaataattg tggagaggag agaatgagag |
| 2701 | agtgtggaaa aaaaaagaat aatgacccag cccccgccct ctgcccccag ctgctcctcg |
| 2761 | cagttcggtt aattggttaa tcacttaacc tgcttttgtc actcggcttt ggctcgggac |
| 2821 | ttcaaaatca gtgatgggag taagagcaaa tttcatcttt ccaaattgat gggtaggcta |
| 2881 | gtaataaaat atttaaaaaa aaacattcaa aaacatggcc acatccaaca tttcctcagg |
| 2941 | caattccttt tgattctttt ttcttccccc tccatgtaga agagggagaa agagaggctc |
| 3001 | tgaaagctgc ttctggggga tttcaaggga ctgggggtgc caaccacctc tggccctgtt |
| 3061 | gtgggggtgt cacagaggca gtggcagcaa caaaggattt gaaacttggt gtgttcgtgg |
| 3121 | agccacaggc agacgatgtc aaccttgtgt gagtgtgacg ggggttgggg tggggcggga |
| 3181 | ggccacgggg gaggccgagg caggggctgg gcagagggga gaggaagcac aagaagtggg |
| 3241 | agtgggagag gaagccacgt gctggagagt agacatcccc ctccttgccg ctgggagagc |
| 3301 | caaggcctat gccacctgca gcgtctgagc ggccgcctgt ccttggtggc cgggggtggg |
| 3361 | ggcctgctgt gggtcagtgt gccaccctct gcagggcagc ctgtgggaga agggacagcg |
| 3421 | ggtaaaaaga gaaggcaagc tggcaggagg gtggcacttc gtggatgacc tccttagaaa |
| 3481 | agactgacct tgatgtcttg agagcgctgg cctcttcctc cctccctgca gggtaggggg |
| 3541 | cctgagttga ggggcttccc tctgctccac agaaaccctg ttttattgag ttctgaaggt |
| 3601 | tggaactgct gccatgattt tggccacttt gcagacctgg gactttaggg ctaaccagtt |
| 3661 | ctctttgtaa ggacttgtgc ctcttgggag acgtccaccc gtttccaagc ctgggccact |
| 3721 | ggcatctctg gagtgtgtgg gagtctggga ggcaggtccc gagccccctg tccttcccac |
| 3781 | ggccactgca gtcaccccgt ctgcgccgct gtgctgttgt ctgccgtgag agcccaatca |
| 3841 | ctgcctatac ccctcatcac acgtcacaat gtcccgaatt cccagcctca ccaccccttc |
| 3901 | tcagtaatga ccctggttgg ttgcaggagg tacctactcc atactgaggg tgaaattaag |
| 3961 | ggaaggcaaa gtccaggcac aagaatggga ccccagcctc tcactctcag ttccactcat |
| 4021 | ccaactggga ccctcaccac gaatctcatg atctgattcg gttccctgtc tcctcctccc |
| 4081 | gtcacagatg tgagccaagg cactgctcag ctgtgaccct aggtgtttct gccttgttga |
| 4141 | catggagaga gccctttccc ctgagaaggc ctggcccctt cctgtgctga gcccacagca |
| 4201 | gcagactgga tatcttggtt atcagcggta gcaccaggat ggaagggcaa ggcacccagg |
| 4261 | gcaggcccac agtcccgctg tcccccactt gcaccctagc ttgtagctgc caacctccca |
| 4321 | gacagcccag cccgctgctc agctccacat gcatagtatc aaccctccac acccgacaaa |
| 4381 | ggggaacaca cccccttgga aatggttctt ttcccccagt cccagctgga agccatgctg |
| 4441 | tctgttctgc tggagcagct gaacatatac atagatgttg ccctgccctc cccatctgca |
| 4501 | ccctgttgag ttgtagttgg atttgtctgt ttatgcttgg attcaccaga gtgactatga |
| 4561 | tagtgaaaag aaaaaaaaaa aaaaaaaagg acacatgtat cttaaaatgc ttgtaaagag |
| 4621 | gtttctaacc caccctcacg aggtgtctct cacccccaca ctgggactcg tgtggcctgt |
| 4681 | gtggtgccac cctgctgggg cctcccaagt tttgaaaggc tttcctcagc acctgggacc |
| 4741 | caacagagac cagcttctag cagctaagga ggccgttcag ctgtgacgaa ggcctgaagc |
| 4801 | acaggattag gactgaagcg atgatgtccc cttccctact tccccttggg gctccctgtg |
| 4861 | tcagggcaca gactaggtct tgtggctggt ctggcttgcg gcgcgaggat ggttctctct |
| 4921 | ggtcatagcc cgaagtctca tggcagtccc aaaggaggct tacaactcct gcatcacaag |
| 4981 | aaaaaggaag ccactgccag ctggggggat ctgcagctcc cagaagctcc gtgagcctca |
| 5041 | gccacccctc agactgggtt cctctccaag ctcgccctct ggaggggcag cgcagcctcc |
| 5101 | caccaagggc cctgcgacca cagcagggat tgggatgaat tgcctgtcct ggatctgctc |
| 5161 | tagaggccca agctgcctgc ctgaggaagg atgacttgac aagtcaggag acactgttcc |
| 5221 | caaagccttg accagagcac ctcagcccgc tgaccttgca caaactccat ctgctgccat |
| 5281 | gagaaaaggg aagccgcctt tgcaaaacat tgctgcctaa agaaactcag cagcctcagg |
| 5341 | cccaattcta ccacttctgg tttgggtaca gttaaaggca accctgaggg acttggcagt |
| 5401 | agaaatccag ggcctcccct ggggctggca gcttcgtgtg cagctagagc tttacctgaa |
| 5461 | aggaagtctc tggacccaga actctccacc aagagcctcc ctgccgttcg ctgagtccca |
| 5521 | gcaattctcc taagttgaag ggatctgaga aggagaagga aatgtggggt agatttggtg |
| 5581 | gtggttagag atatgccccc ctcattactg ccaacagtat cggctgcatt tcttcacgca |
| 5641 | cctcggttcc tcttcctgaa gttcttgtgc cctgctcttc agcaccatgg gccttcttat |
| 5701 | acggaaggct ctgggatctc ccccttgtgg gacaggctct tggggccagc ctaagatcat |
| 5761 | ggtttagggt gatcagtgct ggcagataaa ttgaaaaggc acgctggctt gtgatcttaa |
| 5821 | atgaggacaa tccccccagg gctgggcact cctcccctcc cctcacttct cccacctgca |
| 5881 | gagccagtgt ccttgggtgg gctagatagg atatactgta tgccggctcc ttcaagctgc |
| 5941 | tgactcactt tatcaatagt tccatttaaa ttgacttcag tggtgagact gtatcctgtt |
| 6001 | tgctattgct tgttgtgcta tggggggagg ggggaggaat gtgtaagata gttaacatgg |
| 6061 | gcaaagggag atcttggggt gcagcactta aactgcctcg taaccctttt catgatttca |
| 6121 | accacatttg ctagagggag ggagcagcca cggagttaga ggcccttggg gtttctcttt |
| 6181 | tccactgaca ggctttccca ggcagctggc tagttcattc cctccccagc caggtgcagg |
| 6241 | cgtaggaata tggacatctg gttgctttgg cctgctgccc tctttcaggg gtcctaagcc |
| 6301 | cacaatcatg cctccctaag accttggcat ccttccctct aagccgttgg cacctctgtg |
| 6361 | ccacctctca cactggctcc agacacacag cctgtgcttt tggagctgag atcactcgct |
| 6421 | tcacccacct catctttgtt ctccaagtaa agccacgagg tcggggcgag ggcagaggtg |
| 6481 | atcacctgcg tgtcccatct acagacctgc agcttcataa aacttctgat ttctcttcag |
| 6541 | ctttgaaaag ggttaccctg ggcactggcc tagagcctca cctcctaata gacttagccc |
| 6601 | ctttgagtag ccatgttgag caggactatt tctggcactt gcaaatccca tgatacttct |
| 6661 | ggtaattagt agggtggggg gagggacatg aaatcatatt agcttagctt tctgtctgtg |
| 6721 | aatgtctata taatgtattg tgttgattaa caaatgattt acactaactg ttgctgtaaa |
| 6781 | agtgaatttg gaaataaagt tattactctg attaaa |

Homo sapiens microtubule-associated protein tan isoform 6 (NP_001116538.2)
(SEQ ID NO: 306)
MAEPRQEFEV MEDHAGTYGL GDRKDQGGYT MHQDQEGDTD
AGLKESPLQT PTEDGSEEPG SETSDAKSTP TAEDVTAPLV DEGAPGKQAA
AQPHTEIPEG TTAEEAGIGD TPSLEDEAAG HVTQEPESGK VVQEGFLREP
GPPGLSHQLM SGMPGAPLLP EGPREATRQP SGTGPEDTEG GRHAPELLKH
QLLGDLHQEG PPLKGAGGKE RPGSKEEVDE DRDVDESSPQ DSPPSKASPA
QDGRPPQTAA REATSIPGFP AEGAIPLPVD FLSKVSTEIP ASEPDGPSVG
RAKGQDAPLE FTFHVEITPN VQKEQAHSEE HLGRAAFPGA PGEGPEARGP
SLGEDTKEAD LPEPSEKQPA AAPRGKPVSR VPQLKARMVS KSKDGTGSDD
KKAKTSTRSS AKTLKNRPCL SPKIHTPGSS DPLIQPSSPA VCPEPPSSPK
YVSSVTSRTG SSGAKEMKLK GADGKTKIAT PRGAAPPGQK GQANATRIPA
KTPPAPKTPP SSATKQVQRR PPPAGPRSER GEPPKSGDRS GYSSPGSPGT
PGSRSRTPSL PTPPTREPKK VAVVRTPPKS PSSAKSRLQT APVPMPDLKN
VKSKIGSTEN LKHQPGGGKV QIINKKIDLS NVQSKCGSKD NIKHVPGGGS
VQIVYKPVDL SKVTSKCGSL GNIHHKPGGG QVEVKSEKLD FKDRVQSKIG
SIDNITHVPG GONKKIETHK LTFRENAKAK IDHGAFIVYK SPVVSGDTSP
RHLSNVSSIG SIDMVDSPQL ATLADEVSAS LAKQGL

The mRNA and protein sequences of the other human tau isoforms can be found in GenBank with the following Accession Nos:
tau isoform 1: NM_016835.4 (mRNA)→NP_058519.3 (protein);
tau isoform 2: NM_005910.5 (mRNA)→NP_005901.2 (protein);
tau isoform 3: NM_016834.4 (mRNA)→NP_058518.1 (protein);
tau isoform 4: NM_016841.4 (mRNA)→NP_058525.1 (protein);
tau isoform 5: NM_001123067.3 (mRNA)→NP_001116539.1 (protein);
tau isoform 7: NM_001203251.1 (mRNA)→NP_001190180.1 (protein);
tau isoform 8: NM_001203252.1 (mRNA)→NP_001190181.1 (protein).

As used herein, human tau protein also encompasses proteins that have over its full length at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any of the tau isoforms. The sequences of murine, cyno, and other animal tau proteins are known in the art.

The term “tau associated disease” includes, but is not limited to, a disease associated with abnormal tau protein expression, secretion, phosphorylation, cleavage, and/or aggregation. Tau-associated diseases include but are not limited to Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, and tuberous sclerosis.

The term “homology” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence.

The term “isolated” refers to altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “treat” or “treatment” refers to both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### Antisense Oligonucleotides

Antisense oligonucleotides (ASOs) are powerful and versatile agents utilized in a growing number of applications including RNA reduction, translation arrest, miRNA inhibition, splicing modulation, and polyadenylation site selection. An antisense oligonucleotide binds a target nucleic acid when a sufficient number of nucleobases of the antisense oligonucleotide can form hydrogen bond with the corresponding nucleobases of the target nucleic acid, and modulates the transcription and/or translation of the target nucleic acid. Thus, the nucleobase sequence of an antisense oligonucleotide is complementary to the nucleobase sequence of a target nucleic acid, e.g., a target genomic sequence, pre-mRNA, or mRNA molecule. Hybridization occurs when hydrogen bonding (e.g., Watson-Crick, Hoogsteen or reversed Hoogsteen hydrogen bonding) forms between the complementary nucleobases of the antisense oligonucleotide and target nucleic acid. Non-complementary nucleobases between an antisense oligonucleotide and a target nucleic acid may be tolerated provided that the antisense oligonucleotide remains able to specifically hybridize to a target nucleic acid.

ASOs can be designed to decrease the expression of a target protein through either RNase H-dependent or RNase H-independent manners (see Watts J K, et al., J Pathol. 2012 January; 226(2): 365-379). When an ASO comprising a contiguous stretch of DNA hybridizes with a target RNA, the DNA-RNA heteroduplex recruit RNase H, which cleaves the target RNA in the duplex and promotes subsequent degradation of the RNA fragments by cellular nucleases. ASOs can also decrease target expression independent of RNAse H by sterically blocking pre-mRNA processing and/or translation of mRNA into protein.

Provided herein are antisense oligonucleotides targeting microtubule-associated protein tau (MAPT). In some embodiments, the antisense oligonucleotides provided herein have a nucleobase sequence complementary to a segment of MAPT genomic DNA, pre-mRNA, or mRNA, with 1, 2, 3, 4, or 5 mismatches. No mismatch is counted between an oligonucleotide and the corresponding target nucleic acid if complete base-pairing occurs (e.g., pairing between A and T, and between C and G). A mismatch occurs when a nucleobase of a first nucleic acid is not capable of pairing with the corresponding nucleobase of a second nucleic acid when the two sequences are maximally aligned. For example, if a position in a first sequence has a nucleobase A, and the corresponding position on the second sequence has a nucleobase (e.g., C or G) that cannot pair with A, it constitutes a mismatch. A mismatch is also counted if a position in one sequence has a nucleobase, and the corresponding position on the other sequence has no nucleobase. A modification to the sugar moiety of a nucleotide or internucleoside linkage is not considered a mismatch. Thus, if one sequence comprises a G, and the corresponding nucleobase of a second sequence comprises a modified C (e.g., 5-methylcytosine), no mismatch would be counted.

In the context of a stretch of nucleic acid, the antisense oligonucleotides provided herein are complementary to a segment of MAPT genomic DNA, pre-mRNA, or mRNA, at least, 70%, 80%, 85%, 86%, 87%, 88%, 89%, 90%, 91%, 92%, 93%, 94%, 95%, 96%, 97%, 98%, 99%, or 100% over the entire length of the segment. Percent complementarity of an antisense oligonucleotide with a target nucleic acid can be determined using routine methods, for example, BLAST programs (basic local alignment search tools) or PowerBLAST programs known in the art (Altschul et al., J. Mol. Biol., 1990, 215, 403 410; Zhang and Madden, Genome Res., 1997, 7, 649 656). In some embodiments, the antisense oligonucleotides provided herein have a nucleobase sequence 100% complementary (i.e., fully complementary) to a segment of MAPT genomic DNA, pre-mRNA, or mRNA. As used herein, “fully complementary” or “100% complementary” refers to each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound.

In some embodiments, antisense oligonucleotides provided herein comprise a nucleobase sequence that is complementary to at least 12 contiguous nucleobases (e.g., 12, 13, 14, 15, 16, 17, or 18 contiguous nucleobases) of any sequence provided in Table 1, with 1, 2, or 3 mismatches. In some embodiments, antisense oligonucleotides provided herein comprise a nucleobase sequence that is 100% complementary to at least 12 contiguous nucleobases (e.g., 12, 13, 14, 15, 16, 17, or 18 contiguous nucleobases) of any sequence provided in Table 1.

The antisense compounds provided herein may also have a defined percent identity to a particular nucleotide sequence, SEQ ID NO, or a portion thereof. As used herein, an antisense oligonucleotide is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense oligonucleotides described herein as well as oligonucleotides having non-identical bases relative to the antisense oligonucleotides provided herein also are contemplated. The non-identical bases may be adjacent to each other or dispersed throughout the antisense oligonucleotides. Percent sequence identity of an antisense oligonucleotide can be calculated according to the number of bases that have identical base pairing relative to the sequence to which it is being compared. Percent sequence identity, can be determined using routine methods, for example, BLAST programs (basic local alignment search tools) or PowerBLAST programs known in the art (Altschul et al., J. Mol. Biol., 1990, 215, 403 410; Zhang and Madden, Genome Res., 1997, 7, 649 656); or by the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

In some embodiments, provided herein are antisense oligonucleotides comprising a nucleobase sequence that has at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any of the nucleobase sequences provided in any of Tables 2-17, wherein C of any of the nucleobase sequences is either cytosine or 5-methylcytosine, and wherein at least one nucleotide of the oligonucleotide has a 2′-modification. In some embodiments, provided herein are antisense oligonucleotides comprising a nucleobase sequence that has at least 90% (e.g., 90%, 91%, 92%, 93%, 94%, 95%, 96%, 97%, 98%, 99%, or 100%) sequence identity to any of the nucleobase sequences provided in any of Tables 2-17. In some embodiments, antisense oligonucleotides targeting MAPT comprise any of the nucleobase sequences provided in any of Tables 2-17. In some embodiments, antisense oligonucleotides targeting MAPT consist of any of the nucleobase sequences provided in any of Tables 2-17.

In some embodiments, the antisense oligonucleotides provided herein are 12 to 30 nucleobases in length. For example, an antisense oligonucleotide targeting MAPT can comprise 12, 13, 14, 15, 16, 17, 18, 19, 20, 21, 22, 23, 24, 25, 26, 27, 28, 29, or 30 nucleobases. In some embodiments, the antisense oligonucleotides targeting MAPT are 12 to 25 nucleobases in length. For example, an antisense oligonucleotide targeting MAPT can comprise 12, 13, 14, 15, 16, 17, 18, 19, 20, 21, 22, 23, 24, or 25 nucleobases. In some embodiments, the antisense oligonucleotides targeting MAPT are 15 to 20 nucleobases in length. For example, an antisense oligonucleotide targeting MAPT can comprise 15, 16, 17, 18, 19, or 20 nucleobases. In some embodiments, the antisense oligonucleotides targeting MAPT comprise 17 nucleobases. It is possible to increase or decrease the length of an antisense oligonucleotide, and/or introduce mismatch bases (e.g., 1, 2, 3, 4, or 5 mismatches) in an antisense oligonucleotide without eliminating its activity.

### Chemical Modification of Antisense Oligonucleotides

Oligonucleotides consist of repeating nucleotide units that are linked together by internucleoside phosphodiester bonds. Each nucleotide is composed of a nucleoside, which comprises a nucleobase linked to a sugar moiety, and one or more phosphate groups covalently linked to the sugar moiety. The phosphodiester bonds are made up of a sugar residue (either ribose for RNA or deoxyribose for DNA, collectively furanose) linked via a glycosidic bond to a purine (guanine and/or adenine) and/or pyrimidine base (thymine and cytosine for DNA; and uracil and cytosine for RNA).

Antisense oligonucleotides provided herein can contain one or more modified nucleotide subunits and/or internucleoside linkages. Chemical modifications to oligonucleotides encompass changes to internucleoside linkages, sugar moieties, nucleobases, and/or backbones. Modifications can improve stability, efficacy, and/or reduce immunogenicity of the antisense oligonucleotides. For example, oligonucleotides can be modified to have increased resistance to nucleases, enhanced binding affinity for nucleic acid target, enhanced cellular uptake, and/or increased inhibitory activity when compared to the unmodified oligonucleotides.

In some embodiments, antisense oligonucleotides provided herein include naturally occurring phosphodiester internucleoside linkages. The phosphodiester linkages can be modified to other phosphorous-containing linkages such as phosphorothioate, phosphotriester, methylphosphonate, or phosphoramidate linkages, or non-phosphorous-containing linkages. In some embodiments, antisense oligonucleotides provided herein include one or more modified internucleoside linkages. In some embodiments, antisense oligonucleotides provided herein include phosphorothioate linkages. In some embodiments, each internucleoside linkage of an antisense oligonucleotide is a phosphorothioate internucleoside linkage.

In some embodiments, antisense oligonucleotides provided herein include chemically modified sugar moieties. For example, the antisense oligonucleotides can include 2′ modification on the furanose ring, bridging of non-geminal ring atoms to form bicyclic nucleic acids (BNA), replacement of the sugar ring oxygen atom with other atoms, or combinations thereof. In some embodiments, each nucleotide of an antisense oligonucleotide has a 2′-modified furanose ring. Exemplary 2′-modification include 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In some embodiments, each nucleotide of an antisense oligonucleotide has 2′-O-MOE modification to the sugar moiety.

In some embodiments, the antisense oligonucleotides provided herein can include substitution of a nucleotide at a given position with a modified version of the same nucleotide. For example, a nucleotide (A, G, C or T) can be replaced by the corresponding hypoxanthine, xanthine, 4-acetylcytosine, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, beta-D-mannosylqueosine, 2-methylthio-N6-isopentenyladenine, wybutoxosine, queosine, 2-thiocytosine, or 2,6-diaminopurine.

In some embodiments, the antisense oligonucleotides provided herein include chemically modified oligonucleotides that reduce the immunogenicity of oligonucleotides. For example, oligonucleotides containing 5-methylcytosine or 2′-O-MOE modifications have been shown to exhibit decreased immune stimulation in mice (Henry S. et al., J Pharmacol Exp Ther. 2000 February; 292(2):468-79). In some embodiments, antisense oligonucleotides provided herein include 5-methylcytosines instead of cytosines. In some embodiments, antisense oligonucleotides provided herein include 2′-O-MOE modifications. In some embodiments, antisense oligonucleotides provided herein include 5-methylcytosines and 2′ MOE modifications.

In some embodiments, the antisense oligonucleotides targeting MAPT provided herein include a C6 linker at the 3′ end with the following structure:

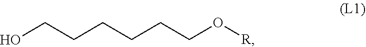

which is attached to the 3′ end of the oligonucleotide via a phosphate bridge, wherein R=PO2-O-oligonucleotide (for phosphodiester internucleoside linkages) or R=POS—O-oligonucleotide (for phosphorothioate internucleoside linkages). Such a 3′ C6 linker can block 3′-exonuclease attack and therefore enhance the stability and duration of effect of the antisense oligonucleotides (see WO 2005/021749 for similar strategy applied to siRNA). In some cases, the 3′ C6 linker can also facilitate synthesis and/or purification of the antisense oligonucleotides. In some embodiments, the antisense oligonucleotides targeting MAPT can have any of the following structures:

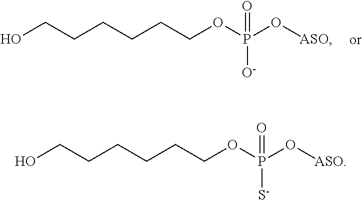

In some embodiments, antisense oligonucleotides provided herein can include an alternative backbone, for example, morpholino, locked nucleic acid (LNA), unlocked nucleic acid (UNA), threose nucleic acid (TNA), glycol nucleic acid (GNA), and/or peptide nucleic acid (PNA). In some embodiments, antisense oligonucleotides provided herein can include bicyclic nucleoside (BNA) comprising a bridge connecting two carbon atoms of the sugar ring. For example, such BNA can include “constrained ethyl” (or “cEt”), comprising a 4′-CH(CH3)-O-2′ bridge connecting the 4′-carbon and the 2′-carbon of the sugar moiety. In some embodiments, antisense oligonucleotides provided herein can include locked nucleic acid (LNA) comprising a bridge connecting two carbon atoms between the 4′ and 2′ position of the nucleoside sugar unit. Such LNA can include α-L-methyleneoxy (4′-CH2-O-2′) LNA, β-D-methyleneoxy (4′-CH₂—O-2′) LNA, ethyleneoxy (4′-(CH₂)₂—O-2′) LNA, aminooxy (4′-CH₂—O—N(R)-2′) LNA, oxyamino (4′-CH₂—N(R)—O-2′) LNA, or any other LNA described in U.S. Pat. Nos. 7,053,207; 6,268,490; 6,770,748; 6,794,499; 7,034,133; 6,525,191; 7,696,345; 7,569,575; 7,314,923; 7,217,805; 7,084,125; or 6,670,461; Patent publication Nos. WO 98/39352 or WO 99/14226. Other suitable LNA include the LNAs described in Braasch et al., Chem. Biol. 8:1-7, 2001; Elayadi et al., Curr. Opinion Invens. Drugs 2: 558-561, 2001; Frieden et al., Nucleic Acids Research, 21: 6365-6372, 2003; Koshkin et al., Tetrahedron, 54: 3607-3630, 1998; Morita et al., Bioorganic Medicinal Chemistry, 11: 2211-2226, 2003; Orum et al., Curr. Opinion Mol. Ther. 3: 239-243, 2001; Singh et al., Chem. Commun. 4: 455-456, 1998; Singh et al., J. Org. Chem., 63: 10035-10039, 1998; or Wahlestedt et al., PNAS 97: 5633-5638, 2000.

### Steric Blockers

An antisense oligonucleotide can bind a target nucleic acid and sterically block the access of DNA or RNA binding proteins, transcription factors, splicing factors, ribosome, and/or the translation machinery to the target nucleic acid, and thus decrease the target expression, without activating RNAse H. For example, such steric blockers can reduce the expression of a target protein by hybridizing to sequences surrounding the start codon of the target, blocking intronic branch point sequences, targeting splice sites, bracketing intronic and/or exonic sequences, or targeting regulatory sequences such as exon splicing enhancers. Steric blockers can be designed based on previously determined or predicted intron-exon boundaries and gene structure, and a panel of different antisense oligonucleotides can be generated for blocking the same site. BLAST analyses can be performed for each ASO to minimize off-target hybridization.

Steric blockers can achieve mRNA reduction by exploiting endogenous cellular surveillance pathways that recognize and degrade aberrant mRNAs. One such pathway is nonsense-mediated mRNA decay (NMD), which modulates gene expression and prevents producing potentially toxic proteins from mRNAs. Defects in pre-mRNA processing can result in protein loss-of-function when a premature termination codon (PTC) is introduced and disrupts the open reading frame. Such PTC-containing mRNA can be a substrate for NMD, which involves communication between the translating ribosome and components of the exon junction complex, including the essential NMD factor UPF1, to degrade the RNA by endonuclease and exonuclease activity. ASOs can be rationally designed to cause target mRNA reduction by directing the target mRNA to the NMD pathway. This can be achieved by designing the sequences of steric blockers to be complementary to specific coding exons, intron-exon junctions, or other sequences necessary for proper pre-mRNA processing, to introduce exon skipping, frameshifting, and/or introducing PTC.

In some embodiments, the antisense oligonucleotides targeting MAPT provided herein are steric blockers, e.g., oligonucleotides comprising a nucleobase sequence that has at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any of the nucleobase sequences provided in any of Tables 2-8. In some embodiments, the antisense oligonucleotides targeting MAPT provided herein are steric blockers that comprise any of the nucleobase sequences provided in any of Tables 2-8. In some embodiments, the antisense oligonucleotides targeting MAPT provided herein are steric blockers that consist of any of the nucleobase sequences provided in any of Tables 2-8. As detailed in the examples below, the steric blockers were designed to target tau constitutive exons (e.g., exons 1, 4, 5, 7, 9, 11, 12, 13), sequences bracketing the start codon of MAPT, splice acceptors and donors, splicing branch points, polypyrimidine track-related sequences, or splicing enhancer or inhibitor sequences. Targeting the start codon and exon 1 would potentially block initiation of translation. ASOs that interfere with splicing and/or induce exon skipping would result in frameshifting and/or introducing a downstream premature stop codon, leading to reduction of MAPT mRNA and/or tau protein.

Chemical modifications can be incorporated into steric blockers to improve their stability, efficacy, and/or cellular uptake. Steric blockers can have chemical modification at each nucleotide position or at some selected positions. For example, incorporation of 2′-modification of the sugar ring (such as 2′-O-methoxyethyl, MOE), inclusion of locked nucleic acid (LNA) and/or backbone modifications (such as phosphorothioate) can decrease nuclease degradation and/or increase binding affinity of the antisense oligonucleotides. Besides sugar and/or backbone modifications, steric blockers can be made from oligomers that are quite different from DNA or RNA. Peptide nucleic acid (PNAs) is an oligonucleotide mimic whose nucleobases are linked by amide bonds. Because the amide backbone is uncharged, binding is characterized by high rates of association and high affinity (see Bentin T, Biochemistry. 1996; 35:8863-8869; Smulevitch S V, Nat Biotech. 1996; 14:1700-1704). Phosphorodiamidate morpholino oligomers (commonly called PMOs or “morpholinos”) are another uncharged DNA analogue. PMOs do not bind complementary targets with the high affinities that characterize PNA binding, but have proven to be effective agents inside cells (see Summerton J, Antisense Nucleic Acid Drug Dev. 1997; 7:187-195; Corey D R, Genome Biol. 2001; 2:REVIEWS1015).

In some embodiments, the antisense oligonucleotides targeting MAPT provided herein are steric blockers comprising 2′-modified nucleotides. The 2′-modification can be selected from the group consisting of 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA). In some embodiments, the antisense oligonucleotides targeting MAPT provided herein are steric blockers that have 2′-O-MOE modification at each nucleotide subunit.

In some embodiments, the antisense oligonucleotides targeting MAPT provided herein are steric blockers that have internucleoside phosphodiester or phosphorothioate linkages.

In some embodiments, the antisense oligonucleotides targeting MAPT provided herein are steric blockers that contain backbone modifications that hinder RNase H binding. Such steric blockers can include modified internucleoside linkages, e.g., methyl phosphonate linkage, methyl phosphonothioate linkage, phosphoromorpholidate linkage, phosphoropiperazidate linkage or phosphoramidite linkage. In some embodiments, every other one of the internucleoside linkage may contain a modified phosphate with a 2′ lower alkyl moiety (e.g., C1-C4, linear or branched, saturated or unsaturated alkyl, such as methyl, ethyl, ethenyl, propyl, 1-propenyl, 2-propenyl, and isopropyl) or a combination thereof. In some embodiments, the antisense oligonucleotides targeting MAPT provided herein are steric blockers that include one or more modified internucleoside linkages described in U.S. Pat. No. 5,149,797.

In some embodiments, the steric blockers targeting MAPT provided herein include a C6 linker at the 3′ end with the following structure:

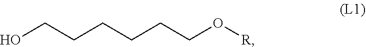

which is attached to the 3′ end of the oligonucleotide via a phosphate bridge, wherein R=PO2-O-oligonucleotide (for phosphodiester internucleoside linkages) or R=POS—O-oligonucleotide (for phosphorothioate internucleoside linkages). Accordingly, the steric blockers targeting MAPT with phosphodiester internucleoside linkages can have the following structure:

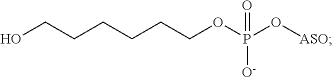

and the steric blockers targeting MAPT with phosphorothioate internucleoside linkages can have the following structure:

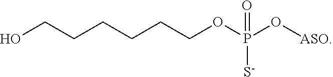

### Gapmers

Antisense oligonucleotides comprising a contiguous stretch of DNA can recruit cellular endonuclease RNase H to the target RNA:DNA heteroduplex and cleave the target RNA in the RNA:DNA duplex. Gapmers are chimeric antisense compounds. Chimeric antisense compounds typically contain at least one region modified so as to confer increased resistance to nuclease degradation, increased cellular uptake, increased binding affinity for the target nucleic acid, and/or increased inhibitory activity, and a second region having nucleotides that are chemically different from the nucleotides of the first region.

Gapmers have a central gap segment consisting of a stretch of contiguous 2′-deoxyribonucleotides, positioned between two wing segments consisting of modified nucleotides on the 5′ and 3′ ends. The gap segment serves as the substrate for endonuclease RNAse H cleavage, while the wing segments with modified nucleotides confer increased resistance to other nuclease degradation. The wing-gap-wing segment can be described as “X-Y-Z,” where “X” represents the length of the 5′ wing, “Y” represents the length of the gap, and “Z” represents the length of the 3′ wing. “X” and “Z” may comprise uniform, variant, or alternating sugar moieties.

In some embodiments, the central gap segment of a gapmer consists of at least five (e.g., 5, 6, 7, 8, 9, 10, 11, 12) contiguous 2′-deoxyribonucleotides; and the 5′ and 3′ wing segments comprise one or more 2′-modified nucleotides. It has been reported that a chimeric oligonucleotide comprising a stretch of up to four contiguous 2′-deoxyribonucleotides does not activate RNAse H. See U.S. Pat. No. 9,157,081. In some embodiments, the antisense oligonucleotides targeting MAPT provided herein are gapmers comprising at least seven (e.g., 7, 8, 9, 10, 11, 12) contiguous 2′-deoxyribonucleotides. In some embodiments, the antisense oligonucleotides targeting MAPT provided herein are gapmers comprising ten contiguous 2′-deoxyribonucleotides. The 2′-modification can be selected from the group consisting of 2′-fluoro, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O—N-methylacetamido (2′-O-NMA).

In some embodiments, the gapmers targeting MAPT are 5-10-5 gapmers that are 20 nucleosides in length, wherein the central gap segment comprises ten 2′-deoxynucleosides and is flanked by 5′ and 3′ wing segments each comprising five nucleosides each with a 2′-modification. Other suitable gapmers include, but are not limited to 5-9-5 gapmers, 5-8-5 gapmers, 4-8-6 gapmers, 6-8-4 gapmers, or 5-7-6 gapmers.

In some embodiments, the antisense oligonucleotides targeting MAPT provided herein are gapmers, e.g., oligonucleotides comprising a nucleobase sequence that has at least 70% (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any of the nucleobase sequences provided in any of Tables 9-15 and 17. In some embodiments, the antisense oligonucleotides targeting MAPT provided herein are gapmers that comprise any of the nucleobase sequences provided in any of Tables 9-15 and 17. In some embodiments, the antisense oligonucleotides targeting MAPT provided herein are gapmers that consist of any of the nucleobase sequences provided in any of Tables 9-15 and 17. As detailed in the examples below, the gapmers were designed to target sequences surrounding the start codon, exon 1, or 3′ untranslated region (UTR) of MAPT transcript. In some embodiments, the gapmers were designed to target 3′ UTR.

In some embodiments, the antisense oligonucleotides targeting MAPT provided herein are 5-10-5 gapmers that comprise any of the nucleobase sequences provided in any of Tables 9-15 and 17, wherein the first to fifth nucleotides each comprise a 2′-O-MOE modified nucleoside, wherein the sixth to fifteenth nucleotides each comprise a 2′-deoxynucleoside, and wherein the sixteenth to twentieth nucleotides each comprise a 2′-O-MOE modified nucleoside.

### MAPT Genomic Sequences Targeted by Antisense Oligonucleotides

In some embodiments, antisense oligonucleotides are designed to target a particular region of MAPT genomic sequence (GenBank Accession No. NG_007398.1; SEQ ID NO: 304) or a region of the corresponding tau mRNA or transcript (SEQ ID NO: 306). The MAPT intron and exon sequences and branch points were determined based on Ensembl genomes database website using Transcript: MAPT-203 ENST00000344290. Screening of exons, introns, and intron/exon junctions of human MAPT gene revealed that targeting some regions in MAPT gene or transcript by antisense oligonucleotides is more effective to reduce tau expression than targeting other regions. For example, Table 1 lists sequences of some preferred regions in MAPT gene or transcript that can be targeted by antisense oligonucleotides.

In some embodiments, antisense oligonucleotides provided herein comprise a nucleobase sequence that is complementary to at least 12 contiguous nucleobases (e.g., 12, 13, 14, 15, 16, 17, or 18 contiguous nucleobases) ofany one of SEQ ID NOs: 487-506, with 1, 2, or 3 mismatches. In some embodiments, antisense oligonucleotides provided herein comprise a nucleobase sequence that is 100% complementary to at least 12 contiguous nucleobases (e.g., 12, 13, 14, 15, 16, 17, or 18 contiguous nucleobases) of any one of SEQ ID NOs: 487-506.

TABLE 1
Selected MAPT genomic, mRNA or premRNA
sequences targeted by tau antisense
oligonucleotides

| Selected MAPT | | | Correspond- |
|---|---|---|---|
| genomic | | | ing MAPT |
| sequences | | | mRNA or |
| (antisense | | | pre-mRNA |
| strand) | SEQ | | sequence |
| targeted by | ID | | targeted |
| tau ASO | NO | Location | by tau ASO |
| ACGCTGGCCTGAA | 292 | Intron/ | GUCCACUAACCUUUCA |
| AGGTTAGTGGAC | | exon 1 | GGCCAGCGU |
| | | junction | (SEQ ID NO: 503) |
| AAAAGCCAAGGTA | 293 | Intron/ | GCAUCGUCAGCUUACC |
| AGCTGACGATGC | | exon 5 | UUGGCUUUU |
| | | junction | (SEQ ID NO: 504) |
| TTTTATATTTTAT | 294 | Intron/ | CCAUGCGAGCUGAUAA |
| CAGCTCGCATGG | | exon 5 | AAUAUAAAA |
| | | junction | (SEQ ID NO: 505) |
| ACCCACAAGCTGA | 295 | Exon 13 | CGGAAGGUCAGCUUGU |
| CCTTCCG | | | GGGU |
| | | | (SEQ ID NO: 487) |
| ACCAGCTGAAGAA | 296 | Exon 4 | GUGUCUCCAAUGCCUG |
| GCAGGCATTGGAG | | | CUUCUUCAGCUGGU |
| ACAC | | | (SEQ ID NO: 488) |
| CTCTCATCTCCAG | 297 | Intron/ | GACUAUUUGCACCUGG |
| GTGCAAATAGTC | | exon II | AGAUGAGAG |
| | | junction | (SEQ ID NO: 506) |
| ATAGTCTACAAAC | 298 | Exon 11 | UCAACUGGUUUGUAGA |
| CAGTTGA | | | CUAU |
| | | | (SEQ ID NO: 489) |
| ATTAGGCAACATC | 299 | Exon 11 | UAUGAUGGAUGUUGCC |
| CATCATA | | | UAAU |
| | | | (SEQ ID NO: 490) |
| GAACCAGGATGGC | 300 | Exon 1 | GGGCUCAGCCAUCCUG |
| TGAGCCC | | | GUUC |
| | | | (SEQ ID NO: 491) |
| CGTCCCTGGCGGA | 301 | Exon 12 | UUUCCUCCGCCAGGGA |
| GGAAA | | | CG |
| | | | (SEQ ID NO: 492) |
| TGGTCAGTAAAAG | 302 | Exon 5 | GUCUUUGCUUUUACUG |
| CAAAGAC | | | ACCA |
| | | | (SEQ ID NO: 493) |
| CTGGAAGCGATGA | 303 | Exon 5 | UUUUUUGUCAUCGCUUC |
| CAAAAAA | | | CAG |
| | | | (SEQ ID NO: 494) |
| CCTTGCTCAGGTC | 479 | Exon 12 | ACCAGUUGACCUGAGC |
| AACTGGT | | | AAGG |
| | | | (SEQ ID NO: 495) |
| GGTTGACATCGTC | 480 | 3′UTR | ACAGGCAGACGAUGUC |
| TGCCTGT | | | AACC |
| | | | (SEQ ID NO: 496) |
| GTCCCACTCTTGT | 481 | 3′UTR | UCCAGGCACAAGAGUG |
| GCCTGGA | | | GGAC |
| | | | (SEQ ID NO: 497) |
| GACATCGTCTGCC | 482 | 3′UTR | AGCCACAGGCAGACGA |
| TGTGGCT | | | UGUC |
| | | | (SEQ ID NO: 498) |
| CCCACTCTTGTGC | 483 | 3′UTR | AGUCCAGGCACAAGAG |
| CTGGACT | | | UGGG |
| | | | (SEQ ID NO: 499) |
| GTCCCAGGTCTGC | 484 | 3′UTR | CCACUUUGCAGACCUG |
| AAAGTGG | | | GGAC |
| | | | (SEQ ID NO: 500) |
| GTCTGCCTGTGGC | 485 | 3′UTR | UCGUGGACrCCACAGGC |
| TCCACGA | | | AGAC |
| | | | (SEQ ID NO: 501) |
| AGTCACTCTGGTG | 486 | 3′UTR | UUGGAUUCACCAGAGUG |
| AATCCAA | | | ACU |
| | | | (SEQ ID NO: 502) |

### Antisense Oligonucleotide Conjugates

Conjugation of antisense oligonucleotides with another moiety can improve the activity, cellular uptake, and/or tissue distribution of the antisense oligonucleotides. For example, antisense oligonucleotides can be covalently linked to one or more diagnostic compound, reporter group, cross-linking agent, nuclease-resistance conferring moiety, lipophilic molecule, cholesterol, lipid, lectin, linker, steroid, uvaol, hecigenin, diosgenin, terpene, triterpene, sarsasapogenin, Friedelin, epifriedelanol-derivatized lithocholic acid, vitamin, biotin, carbohydrate, dextran, dye, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, transferrin, coumarins, phenazine, folate, phenanthridine, anthraquinone, acridine, fluoresceins, and/or rhodamines.

In some embodiments, the antisense oligonucleotides provided herein are attached to a linker molecule. In some embodiments, the antisense oligonucleotides provided herein are linked to lipid or cholesterol. In some embodiments, the antisense oligonucleotides are linked to neutral liposomes (NL) or lipid nanoparticles (LNP). LNPs are self-assembling cationic lipid based systems, which can comprise, for example, a neutral lipid (the liposome base); a cationic lipid (for oligonucleotide loading); cholesterol (for stabilizing the liposomes); and PEG-lipid (for stabilizing the formulation, charge shielding and extended circulation in the bloodstream). Neutral liposomes (NL) are non-cationic lipid based particles.

In some embodiments, the antisense oligonucleotides provided herein are linked to a fatty acid, e.g., an omega-3 fatty acid or omega-6 fatty acid. Suitable omega-3 fatty acids include, e.g., alpha-linolenic acid (ALA), docosahexaenoic acid (DHA), eicosapentaenoic acid (EPA), docosapentaenoic acid (DPA), eicosatetraenoic acid (ETA), eicosatrienoic acid (ETE), eicosapentaenoic acid (EPA), hexadecatrienoic acid (HTA), heneicosapentaenoic acid (HPA), stearidonic acid (SDA), tetracosapentaenoic acid, and tetracosahexaenoic acid.

### Testing Antisense Oligonucleotide Activities

The activity of antisense oligonucleotides can be tested in vitro or in vivo. For in vitro testing, the ASOs can be introduced into cultured cells by transfection or electroporation. Following a period of treatment, MAPT (tau) expression level in the ASO-treated cells can be determined and compared to MAPT (tau) expression level in the untreated control cells.

MAPT expression level can be determined by any appropriate method, for example, by quantitation of MAPT mRNA level, by measuring the quantity of cDNA produced from reverse transcription of MAPT mRNA, or by determining the quantity of tau protein. These methods can be performed on a sample by sample basis or modified for high throughput analysis.

MAPT mRNA level can be detected and quantitated by a probe that specifically hybridizes to a segment of MAPT transcript, e.g., by Northern blot analysis. MAPT mRNA level can also be detected and quantitated by polymerase chain reaction (PCR), using a pair of primers that recognize MAPT transcript. General procedures for PCR are taught in MacPherson et al., PCR: A Practical Approach, (IRL Press at Oxford University Press (1991)). However, PCR conditions used for each application reaction are empirically determined. A number of parameters influence the success of a reaction, for example, annealing temperature and time, extension time, Mg²⁺ and/or ATP concentration, pH, and the relative concentration of primers, templates, and/or deoxyribonucleotides. After amplification, the resulting DNA fragments can be detected by agarose gel electrophoresis followed by visualization with ethidium bromide staining and ultraviolet illumination.

In some embodiments, MAPT mRNA level can be detected and quantitated by quantitative real-time PCR, which monitors the amplification of target nucleic acid by simultaneously incorporating a detectable dye or reporter during the amplification step, using any commercially available real-time PCR system.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example, nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

The activity of antisense oligonucleotides can also be assessed by measuring tau protein levels using the methods known in the art. For example, tau protein level can be quantitated by Western blot analysis (immunoblotting), enzyme-linked immunosorbent assay (ELISA), immunohistochemistry, immunoassays, immunoprecipitation, immunofluorescent assays, immunocytochemistry, fluorescence-activated cell sorting (FACS), radioimmunoassays, immunoradiometric assays, high-performance liquid chromatography (HPLC), mass spectrometry, confocal microscopy, enzymatic assays, or surface plasmon resonance (SPR).

The in vivo activity of antisense oligonucleotides can also be tested in animal models. Testing can be performed in normal animals or in experimental disease animal models. Antisense oligonucleotides can be formulated in a pharmaceutically acceptable diluent and delivered via a suitable administration route. Following a period of treatment, tissue samples, e.g., brain tissue, cerebrospinal fluid (CSF), spinal cord, can be collected and tau expression level can be measured using any of the methods described above. Histological analysis can be performed to evaluate the brain structure and/or the presence of neurofibrillary tangles. Phenotypic changes of the treated animals such as improved cognition or mobility can also be monitored and evaluated.

### Synthesis and Characterization of Oligonucleotides

Single-stranded oligonucleotides can be synthesized using any nucleic acid polymerization methods known in the art, for example, solid-phase synthesis by employing phosphoramidite methodology (S. L. Beaucage and R. P. Iyer, Tetrahedron, 1993, 49, 6123; S. L. Beaucage and R. P. Iyer, Tetrahedron, 1992, 48, 2223), H-phosphonate, phosphortriester chemistry, or enzymatic synthesis. Automated commercial synthesizers can be used, for example, synthesizers from BioAutomation (Irving, Texas), or Applied Biosystems (Foster City, California). In some embodiments, single-stranded oligonucleotides are generated using standard solid-phase phosphoramidite chemistry, such as described in Current Protocols in Nucleic Acid Chemistry, Beaucage, S. L. et al. (Edrs.), John Wiley & Sons, Inc., New York, NY, USA. Phosphorothioate linkages can be introduced using a sulfurizing reagent such as phenylacetyl disulfide or DDTT (((dimethylamino-methylidene)amino)-3H-1,2,4-dithiazaoline-3-thione). It is well known to use similar techniques and commercially available modified amidites and controlled-pore glass (CPG) products such as biotin, fluorescein, acridine or psoralin-modified amidites and/or CPG to synthesize modified oligonucleotides, or fluorescently labeled, biotin, or other conjugated oligonucleotides.

Control of the quality of the starting materials and products from each synthetic step is vital to minimize impurity levels in the final product. However, given the number of synthetic steps per coupling and the number of couplings, presence of impurities is inevitable. Purification methods can be used to exclude the unwanted impurities from the final oligonucleotide product. Commonly used purification techniques for single-stranded oligonucleotides include reverse-phase ion pair high performance liquid chromatography (RP-IP-HPLC), capillary gel electrophoresis (CGE), anion exchange HPLC (AX-HPLC), and size exclusion chromatography (SEC).

After purification, oligonucleotides can be analyzed by mass spectrometry and quantified by spectrophotometry at a wavelength of 260 nm.

### Therapeutic Uses and Methods of Treatment

Provided herein are methods of decreasing tau expression level in a subject, e.g., a human, by administering to the subject a therapeutically effective amount of any of the antisense oligonucleotides described herein. In some embodiments, the antisense oligonucleotide can be administered to the subject through an intrathecal, intracranial, intranasal, intravenous, oral, or subcutaneous route. In some embodiments, such methods further include identifying and selecting a subject who is afflicted with or susceptible to a tau-associated disease.

The antisense oligonucleotides provided herein, or pharmaceutical compositions thereof, can be used to treat or prevent a tau-associated disease in a subject. In some embodiments, the invention provides the antisense oligonucleotides as described herein, or pharmaceutical compositions thereof, for use in the treatment or prevention of a tau-associated disease in a patient. In further embodiments, the invention provides use of the antisense oligonucleotides as described herein in the manufacture of a medicament for use in treatment or prevention of a tau-associated disease in a patient.

Tau-associated diseases include, but are not limited to, Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, and tuberous sclerosis.

### Combination Therapies

The various oligonucleotides described above can be used in combination with other treatment partners. Accordingly, the methods of treating a tau-associated disease described herein can further include administering a second agent to the subject in need of treatment. For example, antisense oligonucleotides targeting microtubule-associated protein tau (MAPT) can be used in combination with an antibody that specifically binds tau protein and/or an agent targeting amyloid beta (Aβ), e.g., an antibody that binds Aβ or a beta-secretase (BACE) inhibitor. In some embodiments, antisense oligonucleotides targeting MAPT are used in combination with an antibody that specifically binds tau protein. In some embodiments, antisense oligonucleotides targeting MAPT are used in combination with a BACE inhibitor.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., an oligonucleotide of the present invention and a combination partner, are both administered to a patient simultaneously in the form of a single entity or dosage. The term “non-fixed combination” means that the therapeutic agents, e.g., an oligonucleotide of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g., synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

### Sample Preparation

Tissue samples can be obtained from a subject treated with antisense oligonucleotide using any of the methods known in the art, e.g., by biopsy or surgery. For example, a sample comprising cerebrospinal fluid can be obtained by lumbar puncture, in which a fine needle attached to a syringe is inserted into the spinal canal in the lumbar area and a vacuum is created such that cerebrospinal fluid may be sucked through the needle and collected in the syringe. CT imaging, ultrasound, or an endoscope can be used to guide this type of procedure.

The sample may be flash frozen and stored at −80° C. for later use. The sample may also be fixed with a fixative, such as formaldehyde, paraformaldehyde, or acetic acid/ethanol. RNA or protein may be extracted from a fresh, frozen or fixed sample for analysis.

### Pharmaceutical Compositions, Dosage, and Administration

Also provided herein are compositions, e.g., pharmaceutical compositions, comprising one or more antisense oligonucleotides provided herein. Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Pharmaceutical compositions are typically formulated to be compatible with its intended route of administration. Examples of routes of administration include intrathecal, intracranial, intranasal, intravenous, oral, or subcutaneous administration.

In some embodiments, the antisense oligonucleotide described herein can be conjugated with an antibody capable of crossing blood-brain barrier (e.g., an antibody that binds transferrin receptor, insulin, leptin, or insulin-like growth factor 1) and be delivered intravenously (Evers et al., Advanced Drug Delivery Reviews 87 (2015): 90-103).

Methods of formulating suitable pharmaceutical compositions are known in the art, see, e.g., Remington: The Science and Practice of Pharmacy. 21st ed., 2005; and the books in the series Drugs and the Pharmaceutical Sciences: a Series of Textbooks and Monographs (Dekker, NY). For example, solutions or suspensions used for parenteral, intradermal, intrathecal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders, for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the therapeutic compounds are prepared with carriers that will protect the therapeutic compounds against rapid elimination from the body, such as a controlled release formulation, including implants and microencapsulated delivery systems.

The pharmaceutical compositions can be included in a container, pack, or dispenser together with instructions for administration.

In non-limiting examples, the pharmaceutical composition containing at least one pharmaceutical agent is formulated as a liquid (e.g., a thermosetting liquid), as a component of a solid (e.g., a powder or a biodegradable biocompatible polymer (e.g., a cationic biodegradable biocompatible polymer)), or as a component of a gel (e.g., a biodegradable biocompatible polymer). In some embodiments, the at least composition containing at least one pharmaceutical agent is formulated as a gel selected from the group of an alginate gel (e.g., sodium alginate), a cellulose-based gel (e.g., carboxymethyl cellulose or carboxyethyl cellulose), or a chitosan-based gel (e.g., chitosan glycerophosphate). Additional, non-limiting examples of drug-eluting polymers that can be used to formulate any of the pharmaceutical compositions described herein include, carrageenan, carboxymethylcellulose, hydroxypropylcellulose, dextran in combination with polyvinyl alcohol, dextran in combination with polyacrylic acid, polygalacturonic acid, galacturonic polysaccharide, polysalactic acid, polyglycolic acid, tamarind gum, xanthum gum, cellulose gum, guar gum (carboxymethyl guar), pectin, polyacrylic acid, polymethacrylic acid, N-isopropylpolyacrylomide, polyoxyethylene, polyoxypropylene, pluronic acid, polylactic acid, cyclodextrin, cycloamylose, resilin, polybutadiene, N-(2-Hydroxypropyl)methacrylamide (HP MA) copolymer, maleic anhydrate-alkyl vinyl ether, polydepsipeptide, polyhydroxybutyrate, polycaprolactone, polydioxanone, polyethylene glycol, polyorganophosphazene, polyortho ester, polyvinylpyrrolidone, polylactic-co-glycolic acid (PLGA), polyanhydrides, polysilamine, poly N-vinyl caprolactam, and gellan.

In some embodiments, delivery of antisense oligonucleotide to a target tissue can be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacal 2002; 54(1):3-27).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but are not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population), and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In some embodiments, the antisense oligonucleotide described herein is dissolved in sterile water, saline (e.g., phosphate-buffered saline), or cerebrospinal fluid (CSF) for administration. In some embodiments, the antisense oligonucleotide described herein is administered intrathecally, e.g., by bolus injection at the L3 or L4 disk space or by infusion using an intrathecal pump.

In some embodiments, about 0.001-1000 mg (e.g., about 0.1-800 mg, about 1-600 mg, about 10-500 mg, about 50-450 mg, about 80-300 mg, about 100-200 mg) of the antisense oligonucleotide described herein is administered to a subject in need thereof.

### Kits

Also provided are kits including one or more antisense oligonucleotides described above and instructions for use. Instructions for use can include instructions for diagnosis or treatment of a tau-associated disease. Kits as provided herein can be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

One skilled in the art will recognize many methods and materials similar or equivalent to those described herein, which could be used in the practice of the present invention. Indeed, the present invention is in no way limited to the methods and materials described.

### EXAMPLES

The invention is further described in the following examples, which do not limit the scope of the invention described in the claims.

### Example 1: General Materials and Methods

### Synthesis and Purification of the Antisense Oligonucleotides

Modified antisense oligonucleotides described in this invention were prepared using standard phosphoramidite chemistry on Mermade192 Synthesizer (BioAutomation) for in vitro use and on Mermade 12 (BioAutomation) for in vivo purpose. Phosphoramidites were dissolved in acetonitrile at 0.15 M concentration (0.08 M on Mermade192); coupling was made by activation of phosphoramidites by a 0.5 M solution of 5-Ethlythiotetrazole in acetonitrile (0.25 M on Mermade192). Coupling time was usually between 3-4 minutes. Sulfurization was made by using a 0.2 M solution of Phenylacetyl Disulfide for five minutes. Oxidation was made by a 0.02 M iodine solution in Pyridine (20%)/Water (9.5%)/Tetrahydrofuran (70.5%) for two minutes. A Capping was made using standard capping reagents. Oligonucleotide growing chains were detritylated for the next coupling by 3% dichloroacetic acid in toluene. After completion of the sequences the support-bound compounds were cleaved and deprotected by liquid ammonium hydroxide at 65° C. for 2 hours. The obtained crude solutions were directly purified by HPLC (Akta Explorer). The purified fractions were analyzed by mass spectrometry and quantified by UV according to their extinction coefficient at 260 nM. The pulled fractions are desalted and lyophilized to dryness.

### In Vitro Testing of the Antisense Oligonucleotides

The antisense oligonucleotides were tested in vitro in various cell lines, including, but are not limited to, human cell lines such as Huh7 cells, Hela cells, and SH-SY5Y cells, and COS1 green monkey cell lines. Cells were obtained from commercial vendors (e.g., American Type Culture Collection (ATCC), Manassas, VA), and cultured according to the vendors' instructions.

Antisense oligonucleotides were also tested in human neurons derived from human embryonic stem cells (hESCs), which were obtained from WiCell Research Institute, Inc., located in Madison, Wisconsin, USA. hESC were converted into functional neuronal cells by forced expression of neurogenin-2 (Ngn2), a neuronal lineage-specific transcription factor. Ngn2 construct was delivered into hESCs by using lentiviral delivery for constitutive expression of rtTA and tetracycline-inducible expression of exogenous proteins driven by a tetO promoter. Samples are in compliance with the Guidelines for human Embryonic Stem Cell Research established by the National Council Institute of Medicine of the National Academies (“NAS Guidelines) and the Office for Human Research Protections, Department of Health and Human Services (“DHHS”) regulations for the protection of human subjects (45 CFR Part1Q).

The antisense oligonucleotides were introduced into cultured cells by either transfection or nucleofection, when the cells reached approximately 60-80% confluency in culture. For transfection, the antisense oligonucleotides were mixed with OptiFect™ Transfection Reagent (Life Tech Cat #12579-017) in the appropriate cell culture media to achieve the desired antisense oligonucleotide concentration and an OptiFect™ concentration ranging from 2 to 12 ug/mL per 100 nM antisense oligonucleotide. For nucleofection, the antisense oligonucleotides were introduced into neuroblastoma SH-SY5Y cells with the Amaxa Nucleofector-II device (Lonza, Walkersville, MD). To test ASO efficacy, nucleofection was carried out in 96-well plates. Nucleofector solution SF was selected based on high viability and efficient transfection after preliminary experiments. On the day of nucleofection, 60-80% confluent cultures were trypsinized, and cells plated in each well. hESC derived human neurons were treated by adding 1 or 10 uM antisense oligonucleotides into the medium to be uptaken through passive uptake.

Cells were harvested 24-72 hours after antisense oligonucleotide treatment, at which time tau mRNA or protein were isolated and measured using methods known in the art and as described herein. In general, when treatments were performed in multiple replicates, the data were presented as the average of the replicate treatments. The concentration of antisense oligonucleotide used varied from cell line to cell line. Methods to determine the optimal antisense oligonucleotide concentration for a particular cell line are well known in the art. The antisense oligonucleotides were typically used at concentrations ranging from 1 nM to 1,000 nM when transfected with OptiFect; and at concentrations ranging from 25 nM to 20,000 nM when transfected using nucleofection.

Quantitation of MAPT (tau) mRNA level was accomplished by quantitative real-time PCR using the ViiA7 Real-Time PCR system (Life Technologies) according to the manufacturer's instructions. Prior to real-time PCR, the isolated RNA was subjected to a reverse transcription reaction, which produced complementary DNA (cDNA) that was then used as the substrate for the real-time PCR amplification. The reverse transcription and real-time PCR reactions were performed sequentially in the same sample wells. Fastlane cell multiplex kit (Qiagen Cat #216513) was used to lyse cells in the well and reverse transcribe mRNAs into cDNAs directly from cultured cells without RNA purification. The cDNAs were then used for tau expression analysis using quantitative real-time PCR. The tau mRNA levels determined by real time PCR were normalized using the expression level of a housekeeping gene whose expression level is constant in cells, such as human glyceraldehyde 3-phosphate dehydrogenase (GAPDH), human TATA-box binding Protein (TBP), or human hypoxanthine-guanine phosphoribosyltransferase (HPRT1).

TaqMan gene expression assays were performed for Real-Time Quantitative RT-PCR, following the protocol as described in the QuantiTect Multiplex RT-PCR Kit (Qiagen Cat #204643) using a duplex RT-PCR reaction. Taqman probes specific to Human MAPT (LifeTech AssayID #Hs00902194_m1: FAM-MGB), Human GAPDH (LifeTech AssayID #Hs02758991_g1: VIC-MGB), Human TBP (LifeTech Cat #4326322E) or Human HPRT1 (LifeTech Cat #4333768T) were used. Samples were run on ViiA7 Real-Time PCR system (Life Technologies) following recommended cycling conditions for duplex RT-PCR. All data were controlled for quantity of cDNA input and tau mRNA levels were normalizing to the levels of the endogenous reference gene. Tau and control gene were amplified in the same reaction with similar, high PCR efficiencies, enabling relative quantification by the ΔΔCT method. Results were presented as percent residual tau mRNA relative to control cells treated with PBS.

### In Vivo Testing of the Antisense Oligonucleotides

Antisense oligonucleotides for MAPT were tested in vivo by delivering the ASOs into the cerebrospinal fluid (CSF) of mice via intracerebral ventricular (ICV) administration. Mice were anesthetized with 5% isoflurane after which isoflurane content in oxygen/nitrous oxygen was reduced to 1.5-2% for maintenance throughout the surgical procedure. Rectal temperature was maintained at 36.9±1.0° C. with homeothermic heating blanked and rectal probe. Anesthetized mice were placed in stereotaxic apparatus and the skin of head was shaved and disinfected with povidone-iodine solution (Betadine). Before incision, mice were given buprenorphine (Temgesic, 0.03 mg/kg, 1 ml/kg, s.c.). Thereafter, incision was made to expose the skull for determination of brain coordinates for the injection. The injection (total volume 2 μl) was given by using 10 μl Hamilton syringe and 28 G needle with micro pump (Harvard Apparatus) into the right lateral ventricle for all animals at following coordinates: AP=+0.5 mm; ML=+1.0 mm; DV=−2.5 mm. Flow rate was 1 μl/min, and needle was left in place for 1 minute after infusion before withdrawn. The skin was closed and the mice were allowed to recover in single cages before returning them to home cage. Additional doses of buprenorphine (Temgesic, 0.03 mg/kg, 1 ml/kg, s.c.) were administered twice a day during the first 48 hours.

Animals were daily monitored by laboratory/animal technicians. The general condition of the animals and wound recovery were monitored; and body weight was measured daily. At the end-point, the animals were deeply anesthetized with sodium pentobarbital (60 mg/kg Mebunat, Orion Pharma, Finland). The mice were subjected to cisterna magna puncture and CSF (3-5 μL per mouse) was collected. Thereafter the mice were subjected to cardiac puncture and blood samples were collected. Approximately 0.4-0.5 mL blood was collected into 500 μL plastic lavender K2EDTA anticoagulant tubes, and centrifuged at 2000 g for 10 min at 4° C. and plasma aliquoted. Thereafter mice were decapitated and brains were collected and dissected into different brain regions such as cortex, hippocampus, and cerebellum. In addition, spinal cords were also collected.

### Example 2: Inhibition of Human Tau Expression in Huh7 Cells by 18Mer 2′-O-MOE Steric Blockers

Antisense oligonucleotide steric blockers were designed to target intron-exon junctions of constitutive exons in human Tau, the invariable exons present in all isoforms. Antisense oligonucleotide steric blockers were designed to induce exon skipping, either by hybridizing to intronic branch point sequences, targeting splice sites directly, bracketing intronic and exonic sequences, exon splicing enhancers sites. The steric blockers targeting MAPT were initially designed as 18 nucleosides in length with 2′-O-(2-methoxyethyl) (2′-O-MOE) ribose sugar modifications in all nucleosides, which act through steric hindrance and do not activate RNase H or RISC. The internucleoside linkages throughout are phosphodiester linkages. An unbiased screen was carried out for the 18-mer uniformly modified 2′-MOE ASOs with phosphodiester backbone. BLAST analyses were performed for each morpholino oligonucleotide sequence to avoid off-target hybridization.

The 18-mer 2′-O-MOE steric blockers targeting tau were tested in vitro for their activity on human Tau mRNA inhibition. Huh7 cells were plated at a density of 10,000 cells per well and transfected using OptiFect reagent (LifeTech Cat #12579-017) with 25 nM of the antisense oligonucleotide. After a treatment period of 48 hours, cDNA was directly prepared from cultured cells using the Fastlane cell multiplex kit (Qiagen Cat #216513). Tau mRNA levels were measured by quantitative real-time PCR in a duplex RT-PCR reaction using Taqman probes specific to Human MAPT (LifeTech AssayID #Hs00902194_m1: FAM-MGB) and Human TBP (TATA-box binding Protein) Endogenous Control (LifeTech Cat #4326322E). All data were controlled for quantity of cDNA input and tau mRNA levels were normalizing to the levels of the endogenous reference gene TBP. Tau and TBP control gene were amplified in the same reaction with similar, high PCR efficiencies, enabling relative quantification by the ΔΔCT method. Results are presented as percent residual Tau mRNA relative to control cells treated with PBS. Table 2 shows the activities of those 18-mer 2′-O-MOE steric blockers in Huh7 cells.

TABLE 2
Inhibition of tau mRNA by 18-mer 2′-O-MOE steric
blockers in Huh7 cells.

| SEQ ID | | % Residual |
|---|---|---|
| NO | ASO Sequence¹ | mRNA² |
| 1 | GTCCACTAACCTTTCAGG | 19.5 |
| 2 | GCATCGTCAGCTTACCTT | 27.7 |
| 3 | TATTTGCACCTGGAGATG | 38.4 |
| 4 | GACTATTTGCACCTGGAG | 40.8 |
| 5 | CATGCGAGCTGATAAAAT | 40.9 |
| 6 | ACCATGCGAGCTGATAAA | 41.3 |
| 7 | CCATGCGAGCTGATAAAA | 43.3 |
| 8 | CGTCAGCTTACCTTGGCT | 44.1 |
| 9 | TGACCATGCGAGCTGATA | 47.4 |
| 10 | ATGCGAGCTGATAAAATA | 52.1 |
| 11 | TTTGCACCTGGAGATGAG | 52.3 |
| 12 | TTGCACCTGGAGATGAGA | 52.4 |
| 13 | ATTTGCACCTGGAGATGA | 61.9 |
| 14 | TCCACTAACCTTTCAGGC | 68.2 |
| 15 | GGTTTCAATCTGCAAGAA | 68.8 |
| 16 | CCACTAACCTTTCAGGCC | 71.3 |
| 17 | CACTAACCTTTCAGGCCA | 72.9 |
| 18 | ACTAACCTTTCAGGCCAG | 76.7 |
| 19 | GCTCAGCCATCCTGGTTC | 77.3 |
| 20 | GTTTCAATCTGCAAGAAG | 80.4 |
| 21 | AGTTCACCTGGGGAAAGA | 85.8 |
| 22 | TTGGAGGTTCACCTGGGA | 85.9 |
| 23 | GGCTACCTGGTTTATGAT | 88.8 |
| 24 | AAAGTTCACCTGGGGAAA | 92.1 |
| 25 | GTTCACTGACCTTGGGTC | 96.8 |
| 26 | CAAAGTTCACCTGGGGAA | 98.4 |
| 27 | CAGCTTACCTTGGCTTTT | 99.8 |
| 28 | GGGCTACCTGGTTTATGA | 101.6 |
| 29 | TCTTCAGCTGGTGTATGT | 103.4 |
| 30 | TTCAAAGTTCACCTGGGG | 103.4 |
| 31 | CCCTTTACCTTTTTATTT | 104.7 |
| 32 | TGCTTCTTCAGCTGGTGT | 106.1 |
| 33 | TCAGCTTACCTTGGCTTT | 106.7 |
| 34 | CTGCTTCTTCAGCTGGTG | 107.9 |
| 35 | GGCCACCTCCTAGAACAC | 108.4 |
| 36 | TCTTACCAGAGCTGGGTG | 108.8 |
| 37 | AAGTTCACCTGGGGAAAG | 109.4 |
| 38 | GTCAGCTTACCTTGGCTT | 109.5 |
| 39 | GGGGCCTGATCACAAACC | 109.7 |
| 40 | AGGTTCACCTGGGAAGGA | 110.2 |
| 41 | GCTTACCTTGGCTTTTTT | 111.4 |
| 42 | TCAAAGTTCACCTGGGGA | 111.7 |
| 43 | CCACTCTCACCTTCCCGC | 112.8 |
| 44 | CCCCCTTTACCTTTTTAT | 113 |
| 45 | GAGGTTCACCTGGGAAGG | 113.3 |
| 46 | GTTCACCTGGGAAGGAAG | 113.6 |
| 47 | CACCTCCTAGAACACAAC | 114.1 |
| 48 | ACTCTCACCTTCCCGCCT | 114.5 |
| 49 | TTCAATCTGCAAGAAGAG | 114.6 |
| 50 | ACTGACCTTGGGTCACGT | 114.7 |
| 51 | TTTCAATCTGCAAGAAGA | 115.1 |
| 52 | TTCTTACCAGAGCTGGGT | 115.5 |
| 53 | CAGGGCTACCTGGTTTAT | 116.1 |
| 54 | GGGCCTGATCACAAACCC | 116.5 |
| 55 | AGGGCTACCTGGTTTATG | 117.2 |
| 56 | CCACCTCCTAGAACACAA | 117.6 |
| 57 | CACTGACCTTGGGTCACG | 118.4 |
| 58 | CCCCTTTACCTTTTTATT | 118.4 |
| 59 | TTCACTGACCTTGGGTCA | 118.6 |
| 60 | GGCCTGATCACAAACCCT | 119.6 |
| 61 | CACTCTCACCTTCCCGCC | 119.7 |
| 62 | CCTGGCCACCTCCTAGAA | 120.4 |
| 63 | CCTTTACCTTTTTATTTC | 120.6 |
| 64 | TCACTGACCTTGGGTCAC | 121.7 |
| 65 | GCCTGATCACAAACCCTG | 122 |
| 66 | CTTTACCTTTTTATTTCC | 122.5 |
| 67 | TTCTTCAGCTGGTGTATG | 124 |
| 68 | GCCACCTCCTAGAACACA | 126.5 |
| 69 | TCTCACCTTCCCGCCTCC | 127.4 |
| 70 | CTTCTTACCAGAGCTGGG | 129.8 |
| 71 | TTCTTCTTACCAGAGCTG | 131.2 |
| 72 | ATCAGCCCCCTGTAAATG | 131.3 |
| 73 | GCTTCTTCAGCTGGTGTA | 133.9 |
| 74 | ACAGGGCTACCTGGTTTA | 134.3 |
| 75 | CTCAGCCATCCTGGTTCA | 134.4 |
| 76 | CAGCCCCCTGTAAATGAA | 136.4 |
| 77 | GGGCTCAGCCATCCTGGT | 137.9 |
| 78 | TCTTCTTACCAGAGCTGG | 139 |
| 79 | CTCTCACCTTCCCGCCTC | 143.1 |
| 80 | TCAGCCCCCTGTAAATGA | 145.9 |
| 81 | CTTCTTCAGCTGGTGTAT | 148 |
| 82 | GGTTCACCTGGGAAGGAA | 153.5 |
| 83 | CATCAGCCCCCTGTAAAT | 156.4 |
| 84 | ACCATCAGCCCCCTGTAA | 157.5 |

¹Each nucleotide has a 2′-O-methoxyethyl (2′-O-MOE) modification; and the internucleoside linkages are phosphodiesters. Each oligonucleotide has a linker (L1) attached to the 3′ end of the ASO via a phosphate bridge, and has the following structure: 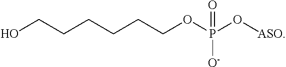
²% Residual mRNA is the level of tau mRNA in the Huh7 cells treated with a single dose of 25 nM of tan ASO for 48 hours as compared to the level of tau mRNA in control Huh7 cells treated with PBS. For example, 19.5% residual mRNA means the ASO of SEQ ID NO: 1 has 80.5% activity in decreasing tau mRNA level.

### Example 3: Inhibition of Human Tau Expression in SH-SY5Y Cells by 18Mer 2′-O-MOE Steric Blockers

Steric blockers that significantly decreased tau expression in Example 2 were selected and tested in human neuroblastoma SH-SY5Y cells. Cultured SH-SY5Y cells were nucleofected with 1,000 nM of a selected antisense oligonucleotide. After a treatment period of approximately 24 hours, cDNA was directly prepared from cultured cells using the Fastlane cell multiplex kit (Qiagen Cat #216513). Tau mRNA levels were measured by quantitative real-time PCR using a duplex RT-PCR reaction, Taqman probes specific to Human MAPT (LifeTech AssayID #Hs00902194_m1: FAM-MGB) and Human GAPDH (LifeTech AssayID #Hs02758991_g1: VIC-MGB) were used. All data were controlled for quantity of cDNA input and Tau mRNA levels were normalizing to the levels of the endogenous reference gene GAPDH. Tau and GAPDH control gene were amplified in the same reaction with similar, high PCR efficiencies, enabling relative quantification by the ΔΔCT method. Results are presented as percent residual Tau mRNA relative to control cells treated with PBS. Table 3 shows the activities of the selected 18-mer 2′-O-MOE steric blockers in SH-SY5Y cells.

TABLE 3
Inhibition of tau mRNA by 18mer 2′-O-MOE steric
blockers in SH-SY5Y cells.

| ASO SEQ ID NO | % Residual mRNA³ |
|---|---|
| 6 | 5.74 |
| 7 | 6.90 |
| 9 | 8.63 |
| 2 | 9.26 |
| 5 | 9.64 |
| 4 | 14.01 |
| 8 | 14.24 |
| 10 | 15.75 |
| 12 | 26.14 |
| 3 | 29.40 |
| 11 | 34.34 |
| 1 | 37.95 |

³% Residual mRNA is the level of tau mRNA in the SH-SY5Y cells treated with a single dose of 1,000 nM of tau ASO for 24 hours as compared to the level of tau mRNA in control cells treated with PBS.

Steric blockers that exhibited significant in vitro inhibition of tau mRNA were tested at different doses. Cultured SH-SY5Y cells were nucleofected with 0.125 nM, 0.25 nM, 0.5 nM, 1,000 nM, 2,000 nM, 4,000 nM and 8,000 nM of one selected antisense oligonucleotide. After a treatment period of approximately 24 hours, cDNA was directly prepared and tau mRNA levels were measured by quantitative real-time PCR as described above. Half maximal inhibitory concentration (IC50) was determined by constructing a dose-response curve and examining the effect of different concentrations of antisense oligonucleotides on reducing Tau mRNA. The IC50 values were calculated by determining the concentration needed to inhibit half of the maximum biological response of the compound and can be used as a measure of the potency of the antisense oligonucleotide. Table 4 shows the IC50 values of the selected 18-mer 2′-O-MOE steric blockers.

TABLE 4
IC50 of selected 18mer 2′-O-MOE steric blockers.

| ASO SEQ ID NO | IC50 (nM) |
|---|---|
| 7 | 65 |
| 5 | 88 |
| 6 | 103 |
| 2 | 200 |
| 4 | 288 |
| 10 | 290 |
| 12 | 430 |
| 3 | 490 |
| 11 | 560 |
| 1 | 590 |

### Example 4: Inhibition of Human Tau Expression in SH-SY5Y Cells by 12-25Mer 2′-O-MOE Steric Blockers

Steric blockers that significantly decreased tau expression in Examples 2 and 3 were selected and made to have different lengths from 12 to 25 nucleosides long. These 12-25mer 2′-O-MOE steric blockers were tested in SH-SY5Y cells. Cultured SH-SY5Y cells were nucleofected with 2,000 nM of a selected antisense oligonucleotide. After a treatment period of approximately 24 hours, cDNA was directly prepared and Tau mRNA levels were measured as described above. Table 5 shows the activities of the 12-25mer 2′-O-MOE steric blockers in SH-SY5Y cells.

TABLE 5
Inhibition of tau mRNA by 12-25mer 2′-O-MOE steric blockers in SH-SY5Y cells.

| SEQ | | % | | Tau |
|---|---|---|---|---|
| ID | | Residual | Length | exon |
| NO | ASO Sequence⁴ | mRNA⁵ | of ASO | targeted |
| 85 | GT^{m}C^{m}CA^{m}CTAA^{m}C^{m}CTTT^{m}CAGG^{m}C^{m}CGTGT^{m}C | 61.7 | 25 | 1 |
| 86 | GT^{m}C^{m}CA^{m}CTAA^{m}C^{m}CTTT^{m}CAGG^{m}C^{m}CGTGT | 61.6 | 24 | 1 |
| 87 | GT^{m}C^{m}CA^{m}CTAA^{m}C^{m}CTTT^{m}CAGG^{m}C^{m}CGTG | 74.7 | 23 | 1 |
| 88 | GT^{m}C^{m}CA^{m}CTAA^{m}C^{m}CTTT^{m}CAGG^{m}C^{m}CGT | 49.2 | 22 | 1 |
| 89 | GT^{m}C^{m}CA^{m}CTAA^{m}C^{m}CTTT^{m}CAGG^{m}C^{m}CG | 60.2 | 21 | 1 |
| 90 | GT^{m}C^{m}CA^{m}CTAA^{m}C^{m}CTTT^{m}CAGG^{m}C^{m}C | 60.6 | 20 | 1 |
| 91 | GT^{m}C^{m}CA^{m}CTAA^{m}C^{m}CTTT^{m}CAGG^{m}C | 67.8 | 19 | 1 |
| 92 | GT^{m}C^{m}CA^{m}CTAA^{m}C^{m}CTTT^{m}CAGG | 61.6 | 18 | 1 |
| 93 | GT^{m}C^{m}CA^{m}CTAA^{m}C^{m}CTTT^{m}CAG | 58.7 | 17 | 1 |
| 94 | GT^{m}C^{m}CA^{m}CTAA^{m}C^{m}CTTT^{m}CA | 65.4 | 16 | 1 |
| 95 | GT^{m}C^{m}CA^{m}CTAA^{m}C^{m}CTTT^{m}C | 64.2 | 15 | 1 |
| 96 | GT^{m}C^{m}CA^{m}CTAA^{m}C^{m}CTTT | 72.5 | 14 | 1 |
| 97 | GT^{m}C^{m}CA^{m}CTAA^{m}C^{m}CTT | 75.3 | 13 | 1 |
| 98 | GT^{m}C^{m}CA^{m}CTAA^{m}C^{m}CT | 87.1 | 12 | 1 |
| 99 | G^{m}CAT^{m}CGT^{m}CAG^{m}CTTA^{m}C^{m}CTTGG^{m}CTTTT | 35.4 | 25 | 5 |
| 100 | G^{m}CAT^{m}CGT^{m}CAG^{m}CTTA^{m}C^{m}CTTGG^{m}CTTT | 35.3 | 24 | 5 |
| 101 | G^{m}CAT^{m}CGT^{m}CAG^{m}CTTA^{m}C^{m}CTTGG^{m}CTT | 37.8 | 23 | 5 |
| 102 | G^{m}CAT^{m}CGT^{m}CAG^{m}CTTA^{m}C^{m}CTTGG^{m}CT | 38.7 | 22 | 5 |
| 103 | G^{m}CAT^{m}CGT^{m}CAG^{m}CTTA^{m}C^{m}CTTGG^{m}C | 50.2 | 21 | 5 |
| 104 | G^{m}CAT^{m}CGT^{m}CAG^{m}CTTA^{m}C^{m}CTTGG | 49.5 | 20 | 5 |
| 105 | G^{m}CAT^{m}CGT^{m}CAG^{m}CTTA^{m}C^{m}CTTG | 42.2 | 19 | 5 |
| 106 | G^{m}CAT^{m}CGT^{m}CAG^{m}CTTA^{m}C^{m}CTT | 25.2 | 18 | 5 |
| 107 | G^{m}CAT^{m}CGT^{m}CAG^{m}CTTA^{m}C^{m}CT | 15.0 | 17 | 5 |
| 108 | G^{m}CAT^{m}CGT^{m}CAG^{m}CTTA^{m}C^{m}C | 10.6 | 16 | 5 |
| 109 | G^{m}CAT^{m}CGT^{m}CAG^{m}CTTA^{m}C | 14.4 | 15 | 5 |
| 110 | G^{m}CAT^{m}CGT^{m}CAG^{m}CTTA | 11.9 | 14 | 5 |
| 111 | G^{m}CAT^{m}CGT^{m}CAG^{m}CTT | 19.6 | 13 | 5 |
| 112 | G^{m}CAT^{m}CGT^{m}CAG^{m}CT | 33.8 | 12 | 5 |
| 113 | GA^{m}CTATTTG^{m}CA^{m}C^{m}CTGGAGATGAGAG | 39.7 | 25 | 11 |
| 114 | GA^{m}CTATTTG^{m}CA^{m}C^{m}CTGGAGATGAGA | 41.1 | 24 | 11 |
| 115 | GA^{m}CTATTTG^{m}CA^{m}C^{m}CTGGAGATGAG | 45.7 | 23 | 11 |
| 116 | GA^{m}CTATTTG^{m}CA^{m}C^{m}CTGGAGATGA | 54.2 | 22 | 11 |
| 117 | GA^{m}CTATTTG^{m}CA^{m}C^{m}CTGGAGATG | 53.2 | 21 | 11 |
| 118 | GA^{m}CTATTTG^{m}CA^{m}C^{m}CTGGAGAT | 63.6 | 20 | 11 |
| 119 | GA^{m}CTATTTG^{m}CA^{m}C^{m}CTGGAGA | 50.6 | 19 | 11 |
| 120 | GA^{m}CTATTTG^{m}CA^{m}C^{m}CTGGAG | 51.0 | 18 | 11 |
| 121 | GA^{m}CTATTTG^{m}CA^{m}C^{m}CTGGA | 38.4 | 17 | 11 |
| 122 | GA^{m}CTATTTG^{m}CA^{m}C^{m}CTGG | 41.2 | 16 | 11 |
| 123 | GA^{m}CTATTTG^{m}CA^{m}C^{m}CTG | 45.6 | 15 | 11 |
| 124 | GA^{m}CTATTTG^{m}CA^{m}C^{m}CT | 46.8 | 14 | 11 |
| 125 | GA^{m}CTATTTG^{m}CA^{m}C^{m}C | 47.5 | 13 | 11 |
| 126 | GA^{m}CTATTTG^{m}CA^{m}C | 56.2 | 12 | 11 |
| 127 | ^{m}C^{m}CATG^{m}CGAG^{m}CTGATAAAATATAAAA | 20.0 | 25 | 5 |
| 128 | ^{m}C^{m}CATG^{m}CGAG^{m}CTGATAAAATATAAA | 14.7 | 24 | 5 |
| 129 | ^{m}C^{m}CATG^{m}CGAG^{m}CTGATAAAATATAA | 24.9 | 23 | 5 |
| 130 | ^{m}C^{m}CATG^{m}CGAG^{m}CTGATAAAATATA | 20.3 | 22 | 5 |
| 131 | ^{m}C^{m}CATG^{m}CGAG^{m}CTGATAAAATAT | 24.3 | 21 | 5 |
| 132 | ^{m}C^{m}CATG^{m}CGAG^{m}CTGATAAAATA | 27.2 | 20 | 5 |
| 133 | ^{m}C^{m}CATG^{m}CGAG^{m}CTGATAAAAT | 23.7 | 19 | 5 |
| 134 | ^{m}C^{m}CATG^{m}CGAG^{m}CTGATAAAA | 24.0 | 18 | 5 |
| 135 | ^{m}C^{m}CATG^{m}CGAG^{m}CTGATAAA | 19.8 | 17 | 5 |
| 136 | ^{m}C^{m}CATG^{m}CGAG^{m}CTGATAA | 17.9 | 16 | 5 |
| 137 | ^{m}C^{m}CATG^{m}CGAG^{m}CTGATA | 23.9 | 15 | 5 |
| 138 | ^{m}C^{m}CATG^{m}CGAG^{m}CTGAT | 87.6 | 14 | 5 |
| 139 | ^{m}C^{m}CATG^{m}CGAG^{m}CTGA | 24.6 | 13 | 5 |
| 140 | ^{m}C^{m}CATG^{m}CGAG^{m}CTG | 23.1 | 12 | 5 |

⁴Each nucleotide has a 2′-O-methoxyethyl (2′-O-MOE) modification; and ^{m}C stands for 5-methylcytosine. The internucleoside linkages are phosphodiesters. Each oligonucleotide has a linker (L1) attached to the 3′ end of the ASO via a phosphate bridge, and has the following structure: 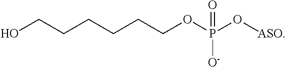
⁵% Residual mRNA is the level of tau mRNA in the SH-SY5Y cells treated with a single dose of 2,000 nM of tau ASO for 24 hours as compared to the level of tau mRNA in control cells treated with PBS.

The IC50 values of selected 12-25mer 2′-O-MOE steric blockers with phosphodiester internucleoside linkages were determined as described above, and shown in Table 6.

A number of 12-25mer 2′-O-MOE steric blockers with phosphorothioate internucleoside linkages were synthesized and the IC50 values of some of those steric blockers were shown in Table 7.

TABLE 6
IC₅₀ of selected 12-25mers 2′-O-MOE steric blockers
with phosphodiester internucleoside linkages.

| ASO SEQ ID NO | IC₅₀ (nM) | Length of ASO |
|---|---|---|
| 103 | 2728 | 21 |
| 105 | 860 | 19 |
| 106 | 1793 | 18 |
| 107 | 838 | 17 |
| 108 | 791 | 16 |
| 109 | 512 | 15 |
| 110 | 728 | 14 |
| 131 | 682 | 21 |
| 133 | 1074 | 19 |
| 134 | 1482 | 18 |
| 135 | 574 | 17 |
| 136 | 544 | 16 |
| 137 | 555 | 15 |
| 138 | 1153 | 14 |
| 117 | 25610 | 21 |
| 120 | 4702 | 18 |
| 121 | 1002 | 17 |
| 122 | 1851 | 16 |
| 123 | 1870 | 15 |
| 124 | 2970 | 14 |

TABLE 7
IC₅₀ of selected 12-25mers 2′-O-MOE steric blockers with phosphorothioate
internucleoside linkages.

| SEQ ID NO | ASO sequence⁵ | IC50 (nM) | Length of ASO |
|---|---|---|---|
| 108 | G^{m}CAT^{m}CGT^{m}CAG^{m}CTTA^{m}C^{m}C | 193 | 16 |
| 111 | G^{m}CAT^{m}CGT^{m}CAG^{m}CTT | 353 | 13 |
| 109 | G^{m}CAT^{m}CGT^{m}CAG^{m}CTTA^{m}C | 426 | 15 |
| 107 | G^{m}CAT^{m}CGT^{m}CAG^{m}CTTA^{m}C^{m}CT | 579 | 17 |
| 140 | ^{m}C^{m}CATG^{m}CGAG^{m}CTG | 877 | 12 |
| 139 | ^{m}C^{m}CATG^{m}CGAG^{m}CTGA | 930 | 13 |
| 135 | ^{m}C^{m}CATG^{m}CGAG^{m}CTGATAAA | 1201 | 17 |
| 134 | ^{m}C^{m}CATG^{m}CGAG^{m}CTGATAAAA | 1398 | 18 |

⁵Each nucleotide has a 2′-O-methoxyethyl (2′-O-MOE) modification; and ^{m}C stands for 5-methylcytosine. The internucleoside linkages are phosphorothioate. Each oligonucleotide has a linker (L1) attached to the 3′ end of the ASO via a phosphate bridge, and has the following structure:

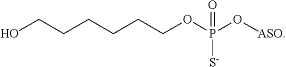

### Example 5: Inhibition of Human Tau Expression in SH-SY5Y Cells by 17Mer 2′-O-MOE Steric Blockers

2′-MO steric blockers that are 17 nucleosides in length were designed to target constitutive exons in human Tau. Those 17mer 2′-O-MOE steric blockers were tested in SH-SY5Y cells. Cultured SH-SY5Y cells were nucleofected with 2,000 nM of a selected antisense oligonucleotide. After a treatment period of approximately 24 hours, cDNA was directly prepared and tau mRNA levels were measured as described above. Table 8 shows the activities of the 17mer 2′-O-MOE steric blockers in SH-SY5Y cells.

TABLE 8
Inhibition of tau mRNA by 17mer MOE steric blockers in SH-SY5Y cells

| SEQ ID | | % Residual | Tau Exon |
|---|---|---|---|
| NO | ASO Sequence⁶ | mRNA⁷ | Targeted |
| 141 | AG^{m}C^{m}CAT^{m}C^{m}CTGGT^{m}CAAA | 131.7 | 1 |
| 142 | ^{m}CAG^{m}C^{m}CAT^{m}C^{m}CTGGTTCAA | 173.6 | 1 |
| 143 | T^{m}CAG^{m}C^{m}CAT^{m}C^{m}CTGGTT^{m}CA | 148.3 | 1 |
| 144 | ^{m}CT^{m}CAG^{m}C^{m}CAT^{m}C^{m}CTGGTT^{m}C | 127.7 | 1 |
| 145 | GG^{m}C^{m}CAG^{m}CGT^{m}C^{m}CGTGT^{m}CA | 115.9 | 1 |
| 146 | G^{m}C^{m}CAG^{m}CGT^{m}C^{m}CGTGT^{m}CA^{m}C | 103.6 | 1 |
| 147 | ^{m}C^{m}CAG^{m}CGT^{m}C^{m}CGTGT^{m}CA^{m}C^{m}C | 96.5 | 1 |
| 148 | ^{m}CAG^{m}CGT^{m}C^{m}CGTGT^{m}CA^{m}C^{m}C^{m}C | 121.9 | 1 |
| 149 | GT^{m}CT^{m}C^{m}CAATG^{m}C^{m}CTG^{m}CTT | 125.7 | 4 |
| 150 | TGT^{m}CT^{m}C^{m}CAATG^{m}C^{m}CTG^{m}CT | 120.5 | 4 |
| 151 | GTGT^{m}CT^{m}C^{m}CAATG^{m}C^{m}CTG^{m}C | 43.5 | 4 |
| 152 | GGTGT^{m}CT^{m}C^{m}CAATG^{m}C^{m}CTG | 114.1 | 4 |
| 153 | T^{m}CA^{m}CGTGA^{m}C^{m}CAG^{m}CAG^{m}CT | 105.1 | 4 |
| 154 | ^{m}CA^{m}CGTGA^{m}C^{m}CAG^{m}CAG^{m}CTT | 109.1 | 4 |
| 155 | A^{m}CGTGA^{m}C^{m}CAG^{m}CAG^{m}CTT^{m}C | 114.6 | 4 |
| 156 | ^{m}CGAAG^{m}CTG^{m}CTGGT^{m}CA^{m}CG | 135.5 | 4 |
| 157 | TTG^{m}CTTTTA^{m}CTGA^{m}C^{m}CA | 18.9 | 5 |
| 158 | ^{m}CTTTG^{m}CTTTTA^{m}CTGA^{m}C^{m}C | 6.8 | 5 |
| 159 | T^{m}CTTTG^{m}CTTTTA^{m}CTGA^{m}C | 14.2 | 5 |
| 160 | GT^{m}CTTTG^{m}CTTTTA^{m}CTGA | 68.2 | 5 |
| 161 | TTTTTTGT^{m}CATCG^{m}CTT^{m}C | 18.5 | 5 |
| 162 | TTTTTGT^{m}CATCG^{m}CTT^{m}C^{m}C | 20.0 | 5 |
| 163 | TTTTGT^{m}CATCG^{m}CTT^{m}C^{m}CA | 24.4 | 5 |
| 164 | TTTGT^{m}CATCG^{m}CTT^{m}C^{m}CAG | 30.5 | 5 |
| 165 | AT^{m}CTT^{m}CGTTTTA^{m}C^{m}CAT^{m}C | 110.1 | 7 |
| 166 | GAT^{m}CTT^{m}CGTTTTA^{m}C^{m}CAT | 111.2 | 7 |
| 167 | ^{m}CGAT^{m}CTT^{m}CGTTTTA^{m}C^{m}CA | 108.4 | 7 |
| 168 | G^{m}CGAT^{m}CTT^{m}CGTTTTA^{m}C^{m}C | 131.1 | 7 |
| 169 | TGGGTGGTGT^{m}CTTTGGA | 104.6 | 7 |
| 170 | GGGTGGTGT^{m}CTTTGGAG | 101.6 | 7 |
| 171 | GGTGGTGT^{m}CTTTGGAG^{m}C | 105.3 | 7 |
| 172 | GTGGTGT^{m}CTTTGGAG^{m}CG | 107.4 | 7 |
| 173 | AT^{m}C^{m}C^{m}C^{m}CTGATTTTGGAG | 130.3 | 9 |
| 174 | GAT^{m}C^{m}C^{m}C^{m}CTGATTTTGGA | 117.8 | 9 |
| 175 | ^{m}CGAT^{m}C^{m}C^{m}C^{m}CTGATTTTGG | 99.7 | 9 |
| 176 | G^{m}CGAT^{m}C^{m}C^{m}C^{m}CTGATTTTG | 116.1 | 9 |
| 177 | G^{m}C^{m}CT^{m}C^{m}C^{m}CGG^{m}CCTGGTG^{m}CT | 129.8 | 9 |
| 178 | ^{m}C^{m}CT^{m}C^{m}C^{m}CGG^{m}CCTGGTG^{m}CTT | 135.7 | 9 |
| 179 | ^{m}CT^{m}C^{m}C^{m}CGG^{m}CCTGGTG^{m}CTT^{m}C | 133.8 | 9 |
| 180 | T^{m}C^{m}C^{m}CGG^{m}CCTGGTG^{m}CTT^{m}CA | 153.5 | 9 |
| 181 | A^{m}CTGGTTTGTAGA^{m}CTAT | 32.6 | 11 |
| 182 | AA^{m}CTGGTTTGTAGA^{m}CTA | 51.5 | 11 |
| 183 | ^{m}CAA^{m}CTGGTTTGTAGA^{m}CT | 29.4 | 11 |
| 184 | T^{m}CAA^{m}CTGGTTTGTAGA^{m}C | 28.2 | 11 |
| 185 | TATGATGGATGTTG^{m}C^{m}CT | 41.7 | 11 |
| 186 | ATGATGGATGTTG^{m}C^{m}CTA | 46.4 | 11 |
| 187 | TGATGGATGTTG^{m}C^{m}CTAA | 40.9 | 11 |
| 188 | GATGGATGTTG^{m}C^{m}CTAAT | 53.6 | 11 |
| 189 | TTTTA^{m}CTT^{m}C^{m}CA^{m}C^{m}CTGG^{m}C | 130.4 | 12 |
| 190 | ATTTTA^{m}CTT^{m}C^{m}CA^{m}C^{m}CTGG | 111.2 | 12 |
| 191 | GATTTTA^{m}CTT^{m}C^{m}CA^{m}C^{m}CTG | 119.6 | 12 |
| 192 | AGATTTTA^{m}CTT^{m}C^{m}CA^{m}C^{m}CT | 123.1 | 12 |
| 193 | ATTT^{m}C^{m}CT^{m}C^{m}CG^{m}C^{m}CAGGGA | 78.0 | 12 |
| 194 | TTT^{m}C^{m}CT^{m}C^{m}CG^{m}C^{m}CAGGGA^{m}C | 76.1 | 12 |
| 195 | TT^{m}C^{m}CT^{m}C^{m}CG^{m}C^{m}CAGGGA^{m}CG | 71.5 | 12 |
| 196 | T^{m}C^{m}CT^{m}C^{m}CG^{m}C^{m}CAGGGA^{m}CGT | 89.0 | 12 |
| 197 | AAGGT^{m}CAG^{m}CCTTGTGGGT | 62.1 | 13 |
| 198 | GAAGGT^{m}CAG^{m}CCTTGTGGG | 49.9 | 13 |
| 199 | GGAAGGT^{m}CAG^{m}CCTTGTGG | 59.3 | 13 |
| 200 | ^{m}CGGAAGGT^{m}CAG^{m}CCTTGTG | 51.9 | 13 |
| 201 | A^{m}C^{m}C^{m}CTG^{m}CTTGG^{m}C^{m}CAGGG | 116.5 | 13 |
| 202 | ^{m}C^{m}C^{m}CTG^{m}CTTGG^{m}C^{m}CAGGGA | 106.1 | 13 |
| 203 | ^{m}C^{m}CTG^{m}CTTGG^{m}C^{m}CAGGGAG | 105.3 | 13 |
| 204 | ^{m}CTG^{m}CTTGG^{m}C^{m}CAGGGAGG | 133.6 | 13 |

⁶Each nucleotide has a 2′-O-MOE modification; and ^{m}C stands for 5-methylcytosine. The internucleoside linkages are phosphodiesters. Each oligonucleotide has a linker (L1) attached to the 3′ end of the ASO via a phosphate bridge, and has the following structure: 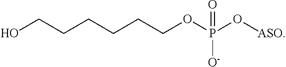
⁷% Residual mRNA is the level of tau mRNA in the SH-SY5Y cells treated with a single dose of 2,000 nM of tau ASO for 24 hours as compared to the level of tau mRNA in control cells treated with PBS.

### Example 6: Inhibition of Human Tau Expression in Huh7 Cells by 5-10-5 Gapmers

Antisense oligonucleotides sequences were designed to be complementary to the shortest Tau isoform, transcript variant 4, mRNA (GenBank: NM_016841.4). BLAST analyses were performed for each oligonucleotide sequence to avoid off-target hybridization. Newly designed modified chimeric antisense oligonucleotides were designed as 5-10-5 gapmers that are 20 nucleosides in length, wherein the central gap segment comprises ten 2′-deoxynucleosides and is flanked by wing segments on the 5′ direction and the 3′ direction comprising five nucleosides each with a 2′-O-MOE ribose sugar modification. The internucleoside linkages throughout each gapmer are phosphorothioate (P═S) linkages.

The gapmers targeting tau were tested for inhibiting human Tau mRNA expression in vitro. Huh7 cells were plated at a density of 10,000 cells per well and transfected using OptiFect reagent (LifeTech Cat #12579-017) with 25 nM of the antisense oligonucleotide. After a treatment period of 48 hours, cDNA was directly prepared from cultured cells using the Fastlane cell multiplex kit (Qiagen Cat #216513). Tau mRNA levels were measured by quantitative real-time PCR in a duplex RT-PCR reaction using Taqman probes specific to Human MAPT (LifeTech AssayID #Hs00902194_m1: FAM-MGB) and Human TBP (TATA-box binding Protein) Endogenous Control (LifeTech Cat #4326322E). All data were controlled for quantity of cDNA input and Tau mRNA levels were normalizing to the levels of the endogenous reference gene TBP. Tau and TBP control gene were amplified in the same reaction with similar, high PCR efficiencies, enabling relative quantification by the ΔΔCT method. Results are presented as percent residual Tau mRNA relative to control cells treated with PBS. Table 9 shows the activities of the gapmers in Huh7 cells.

TABLE 9
Inhibition of Tau mRNA by 5-10-5 MOE gapmers in
Huh7 cells

| SEQ | | % |
|---|---|---|
| ID | | Residual |
| NO | ASO Sequence⁸ | mRNA⁹ |
| 205 | C*C*G*T*A*CGTCCCAGCGT*G*A*T*C* | 72.5 |
| 206 | G*G*C*T*C*AGCCATCCTGG*T*T*C*A* | 26.4 |
| 207 | C*C*C*G*T*ACGTCCCAGCG*T*G*A*T* | 29 |
| 208 | G*G*T*T*G*ACATCGTCTGC*C*T*G*T* | 29.1 |
| 209 | G*G*G*C*T*CAGCCATCCTG*G*T*T*C* | 30.1 |
| 210 | G*G*C*C*A*GCGTCCGTGTC*A*C*C*C* | 32.8 |
| 211 | G*G*C*T*C*TCCCAGCGGCA*A*G*G*A* | 32.8 |
| 212 | C*C*C*T*C*TTGGTCTTGGT*G*C*A*T* | 33.2 |
| 213 | C*G*G*G*A*CCTGCCTCCCA*G*A*C*C* | 33.6 |
| 214 | G*C*T*G*G*TCTCTGTTGGG*T*C*C*C* | 34.5 |
| 215 | G*G*G*C*T*CTCTCCATGTC*A*A*C*A* | 34.6 |
| 216 | G*G*T*C*T*CTGTTGGGFCC*C*A*G*G* | 34.9 |
| 217 | G*G*G*A*C*CTGCCTCCCAG*A*C*C*C* | 35.2 |
| 218 | C*C*C*A*A*CCCGTACGTCC*C*A*G*C* | 37.4 |
| 219 | G*C*T*T*C*GTCTTCCAGGC*T*G*G*G* | 39 |
| 220 | C*C*G*T*G*TCACCCTCTTG*G*T*C*T* | 40.8 |
| 221 | C*T*T*G*G*CTCTCCCAGCG*G*C*A*A* | 40.8 |
| 222 | C*G*G*C*C*TCCTTAGCTGC*T*A*G*A* | 41.7 |
| 223 | C*A*G*C*G*TCCGTGTCACC*C*T*C*T* | 42.8 |
| 224 | G*C*T*C*A*GCCATCCTGGT*T*C*A*A* | 42.9 |
| 225 | C*C*T*G*G*ACTTTGCCTTC*C*C*T*T* | 43.5 |
| 226 | G*T*C*C*C*ACTCTTGTGCC*T*G*G*A* | 44.1 |
| 227 | A*C*C*T*G*GCCACCTCCTG*G*T*T*T* | 45 |
| 228 | T*T*G*G*C*TTTGGCGTTCT*C*G*C*G* | 45.1 |
| 229 | C*G*C*T*T*CCAGTCCCGTC*T*T*T*G* | 46.4 |
| 230 | G*G*T*G*A*TCACCTCTGCC*C*T′T*G* | 46.4 |
| 231 | G*G*T*A*C*CTCCTGCAACC*A*A*C*C* | 47.7 |
| 232 | C*A*C*G*T*GGCTTCCTCTC*C*C*A*C* | 49.4 |
| 233 | G*C*G*T*C*CGTGTCACCCT*C*T*T*G* | 50.6 |
| 234 | C*A*C*C*C*TCTTGGTCTTG*G*T*G*C* | 52.3 |
| 235 | G*T*C*C*C*AGCGTGATCTT*C*C*A*T* | 52.5 |
| 236 | G*C*C*A*G*CACTGATCACC*C*T*A*A* | 53.1 |
| 237 | T*G*G*T*C*TCTGTTGGGTC*C*C*A*G | 53.6 |
| 238 | C*C*G*C*C*TCCCGGCTGGT*G*C*T*T* | 55.6 |
| 239 | G*G*C*C*A*CACGAGTCCCA*G*T*G*T* | 58.2 |
| 240 | G*T*C*C*C*TCAGGGTTGCC*T*T*T*A* | 58.5 |
| 241 | G*G*A*C*C*ACTGCCACCTT*C*T*T*G* | 58.8 |
| 242 | C*A*C*C*T*GGCCACCTCCT*G*G*T*T* | 58.9 |
| 243 | C*C*C*G*C*CTCCCGGCTGG*T*G*C*T* | 59.7 |
| 244 | G*G*T*G*C*CTTGCCCTTCC*A*T*C*C* | 60.3 |
| 245 | C*C*C*G*T*CACACTCACAC*A*A*G*G* | 61.1 |
| 246 | C*C*C*A*A*TCCCTGCTGTG*G*T*C*G* | 61.4 |
| 247 | G*G*G*T*C*CCACTCTTGTG*C*C*T*G* | 62.9 |
| 248 | G*C*T*T*C*CAGTCCCGTCT*T*T*G*C* | 63 |
| 249 | C*C*C*T*T*CTCCCACAGGC*T*G*C*C* | 63.1 |
| 250 | C*T*G*G*T*GCCACCACTGA*C*A*A*C* | 63.2 |
| 251 | G*C*C*A*C*TGCCTCTGIGA*C*A*C*C* | 63.3 |
| 252 | G*T*G*C*C*ACCACTGACAA*C*C*A*A* | 63.5 |
| 253 | C*T*T*G*C*CCTTCCATCCT*G*G*T*G* | 63.7 |
| 254 | G*C*C*T*G*GACTTTGCCTT*C*T*C*T* | 64.3 |
| 255 | G*C*C*T*C*TAACTCCGTGG*C*T*G*C* | 65.1 |
| 256 | G*A*T*C*C*CAGAGCCTTCC*G*T*A*T* | 66.3 |
| 257 | C*A*T*C*C*TCGCGCCGCAA*G*C*C*A* | 66.7 |
| 258 | G*C*C*T*C*CCGGCTGGTGC*T*T*C*A* | 66.8 |
| 259 | G*T*G*C*C*TGGACTTTGCC*T*T*C*C* | 67.1 |
| 260 | C*T*G*C*C*ACTGCCTCTGT*G*A*C*A* | 69.7 |
| 261 | C*C*T*G*G*CCACCTCCTGG*T*T*T*A* | 70.3 |
| 262 | G*G*G*T*G*CCTTGCCCTTC*C*A*T*C* | 71.1 |
| 263 | C*C*A*C*T*CCCACTTCTTG*T*G*C*T* | 71.8 |
| 264 | G*T*G*C*T*TCAGGCCTTCG*T*C*A*C* | 74.6 |
| 265 | C*T*G*^{m}C*C*AGCTTGCCTTC*T*C*T*T* | 75.9 |
| 266 | C*T*C*C*C*GGCTGGTGCTT*C*A*G*G* | 76.2 |
| 267 | C*G*C*C*T*CCCGGCTGGFG*C*T*T*C* | 78 |
| 268 | C*T*G*G*C*CACCTCCTGGT*T*T*A*T* | 78.7 |
| 269 | G*G*C*C*A*CCTCCTGGTTT*A*T*G*A* | 79.1 |
| 270 | C*C*A*T*T*CTGGTGCCACC*A*C*T*G* | 82.9 |
| 271 | C*C*T*G*C*CAGCTTGCCTT*C*T*C*T* | 83.7 |
| 272 | A*A*T*C*C*CTGCTGTGGTC*G*C*A*G* | 83.7 |
| 273 | G*C*C*A*C*CACTGACAACC*A*A*G*A* | 84.1 |
| 274 | C*T*T*G*T*CGGCCATGAT*A*T*A*G* | 87.7 |
| 275 | T*A*A*G*C*AGTGGGTTCTC*T*A*G*T* | 88 |
| 276 | C*C*T*C*C*CGGCTGGTGCT*T*C*A*G* | 88.8 |
| 277 | C*T*C*C*T*GCCAGCTTGCC*T*T*C*T* | 92.2 |
| 278 | C*T*T*C*T*CCTCCGGCCAC*T*A*G*T* | 93.6 |
| 279 | C*T*C*C*T*CCGGCCACTAG*T*G*G*G* | 94.2 |
| 280 | C*C*T*T*C*TCCTCCGGCCA*C*T*A*G* | 94.9 |
| 281 | G*A*G*C*C*TTCTCCTCCGG*C*C*A*C* | 105.7 |
| 282 | G*C*C*T*T*CTCCTCCGGCC*A*C*T*A* | 112.5 |
| 283 | C*C*T*T*A*CCTGCTAGCTG*G*C*G*T* | 128.7 |

⁸The nucleotides with* have a 2′-O-MOE modification; the nucleotides without * are 2′-deoxynucleosides. The interaucleoside linkages are phosphorothioate.
⁹%Residual mRNA is the level of tau mRNA in the Huh7 cells treated with a single dose of 25 nM of tau ASO for 48 hours as compared to the level of tau mRNA in control Huh7 cells treated with PBS.

### Example 7: Inhibition of Human Tau Expression in SH-SY5Y Cells by 5-10-5 Gapmers

Gapmers that significantly decreased Tau mRNA expression in Example 6 were selected and tested in SH-SY5Y cells. Cultured SH-SY5Y cells were nucleofected with 2,000 nM antisense oligonucleotide. After a treatment period of approximately 24 hours, cDNA was directly prepared from cultured cells using the Fastlane cell multiplex kit (Qiagen Cat #216513). Tau mRNA levels were measured by quantitative real-time PCR using a duplex RT-PCR reaction, Taqman probes specific to Human MAPT (LifeTech AssayID #Hs00902194_m1: FAM-MGB) and Human GAPDH (LifeTech AssayID #Hs02758991_g1: VIC-MGB) were used. All data were controlled for quantity of cDNA input and Tau mRNA levels were normalizing to the levels of the endogenous reference gene GAPDH. Tau and GAPDH control gene were amplified in the same reaction with similar, high PCR efficiencies, enabling relative quantification by the ΔΔCT method. Results are presented as percent residual Tau mRNA relative to control cells treated with PBS. Table 10 shows the activities of selected 5-10-5 gapmers in SH-SY5Y cells. The IC50 values of selected 5-10-5 gapmers with 5-methylcytosines were determined in SH-SY5Y cells as described above, and shown in Table 11.

TABLE 10
Inhibition of Tau mRNA by 5-10-5 gapmers in SH-SY5Y cells

| ASO SEQ ID NO | % Residual mRNA¹⁰ |
|---|---|
| 204 | 3.0 |
| 205 | 7.6 |
| 202 | 10.7 |
| 203 | 15.3 |
| 208 | 19.2 |
| 207 | 24.6 |
| 209 | 34.3 |
| 210 | 46.9 |
| 206 | 51.4 |
| 211 | 62.1 |
| 201 | 83.2 |

¹⁰% Residual mRNA is the level of tau mRNA in the SH-SY5Y cells treated with a single dose of 2,000 nM of tau ASO for 24 hours as compared to the level of tau mRNA in control cells treated with PBS.

TABLE 11
IC50 of selected 5-10-5 gapmers with 5-
methylcytosine in SH-SY5Y cells

| SEQ | | |
|---|---|---|
| ID | | IC₅₀ |
| NO | ASO Sequence¹¹ | (nM) |
| 284 | ^{m}C*^{m}C*G*T*A*^{m}CGT^{m}C^{m}C^{m}CAG^{m} | 331 |
| | CGT*G*A*T*^{m}C* | |
| 285 | G*G*T*T*G*A^{m}CAT^{m}CGT^{m}CTG^{m} | 170 |
| | C*^{m}C*T*G*T* | |
| 286 | G*G*G*^{m}C*T*^{m}CAG^{m}C^{m}CAT^{m}C^{m}C | 268 |
| | TG*G*T*T*^{m}C* | |
| 287 | G*G*^{m}C*T*^{m}C*T^{m}C^{m}C^{m}CAG^{m}CGG | 78 |
| | ^{m}CA*A*G*G*A* | |
| 288 | ^{m}C*^{m}C*^{m}C*T*^{m}C*TTGGT^{m}CTTGG | 366 |
| | T*G*^{m}C*A*T* | |
| 289 | G*^{m}C*T*G*G*T^{m}CT^{m}cTGTTGGG | 133 |
| | *T*^{m}C*^{m}C*^{m}C* | |
| 290 | G*T*^{m}C*^{m}C*^{m}C*A^{m}CT^{m}CTTGTG^{m} | 458 |
| | C^{m}C*T*G*G*A* | |
| 791 | G*G*G*^{m}C*T*^{m}CT^{m}CT^{m}C^{m}CATGT | 118 |
| | ^{m}C*A*A*^{m}C*A* | |

¹¹The nucleotides with * have a 2′-O-MOE modification; the nucleotides without * are 2′-deoxynucleosides; and ^{m}C stands for 5-methylcytosine. The internucleoside linkages are phosphorothioate.

### Example 8: Characterization of Antisense Oligonucleotide Targeting MAPT

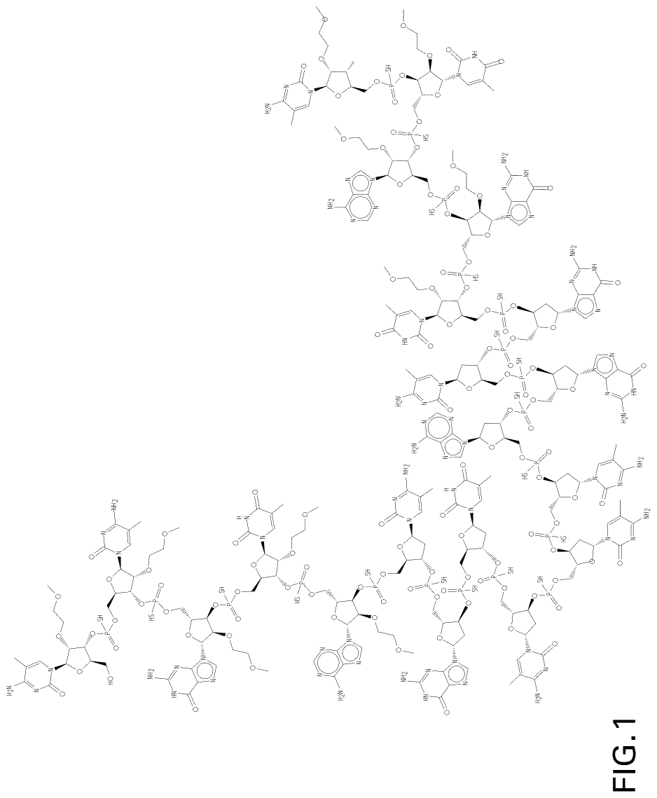

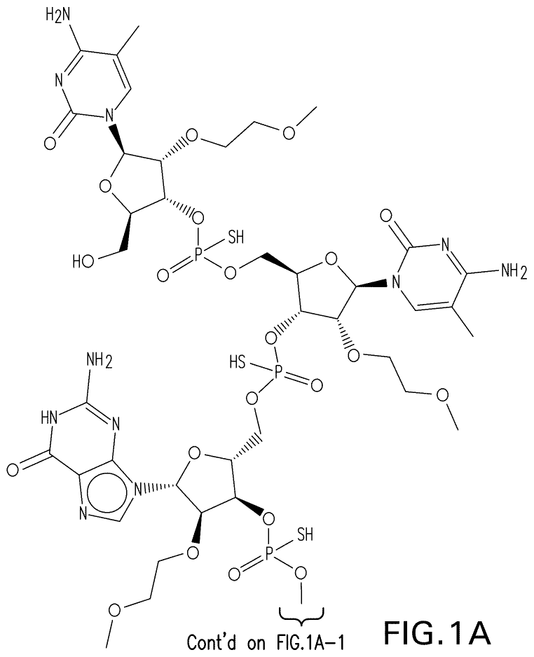

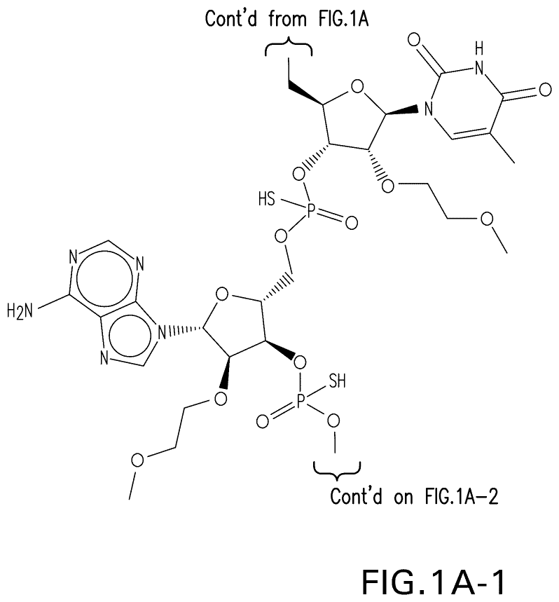

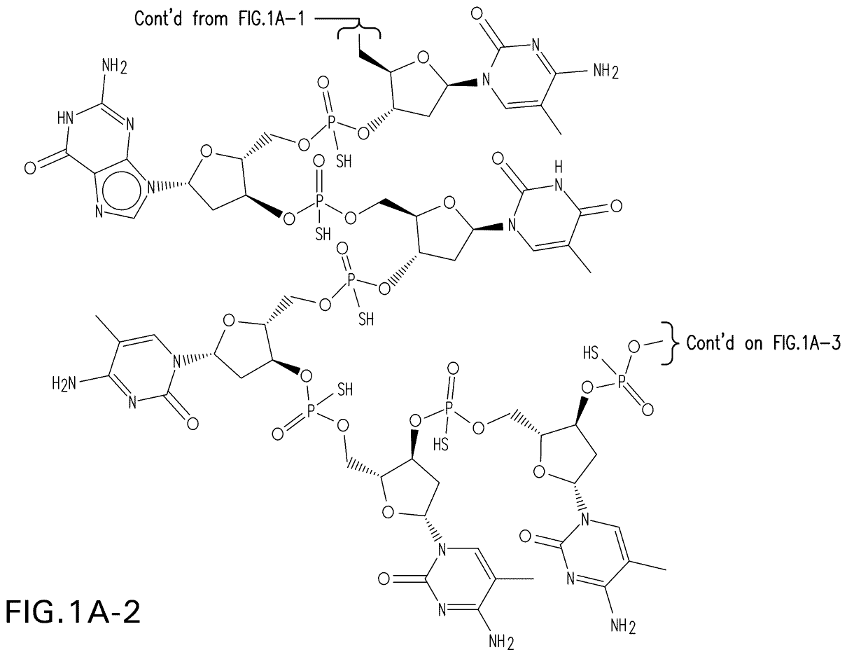

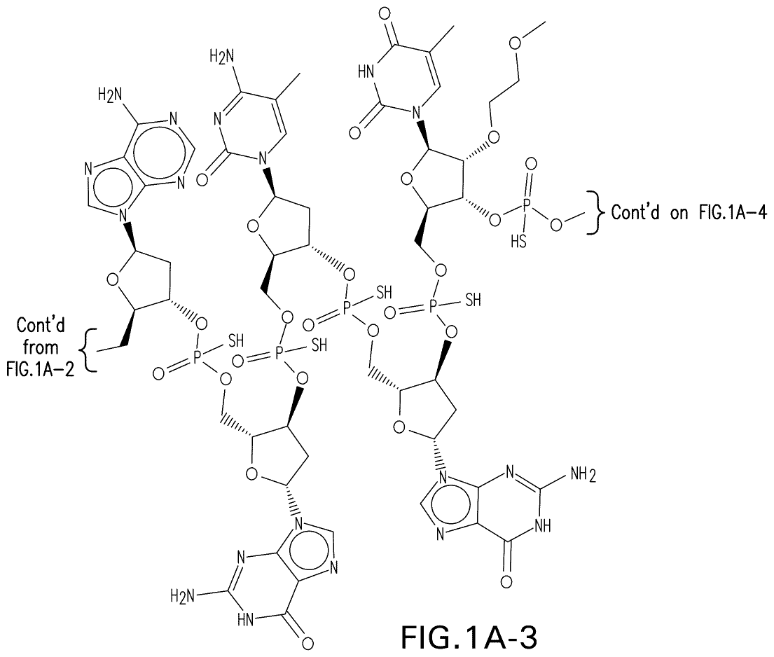

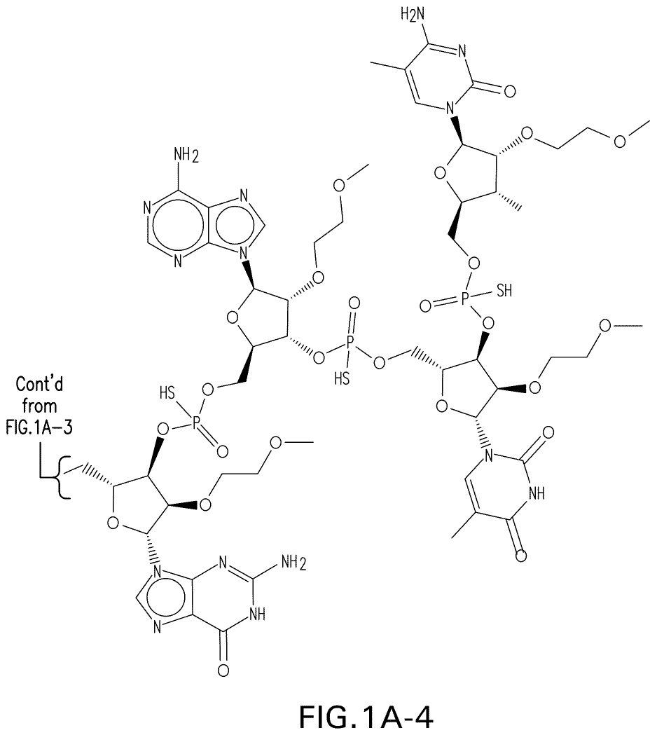

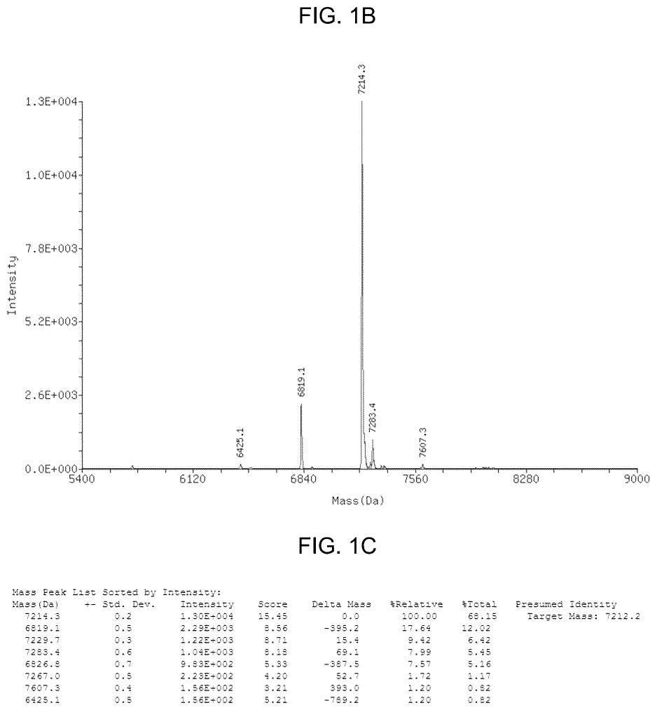

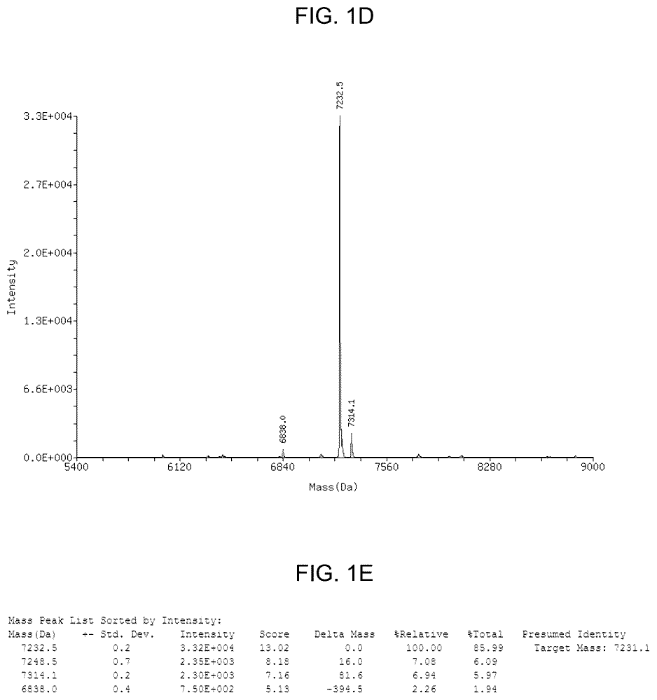

Antisense oligonucleotides targeting MAPT were characterized by using Thermo Scientific high-throughput liquid chromatography-mass spectrometry (LC-MS) instrument. This method was used to confirm the expected masses of antisense oligonucleotide (ASO) and provide information about sample purity and the identification of major components present. For example, the antisense oligonucleotide comprising SEQ ID NO: 284 has the structure shown in FIG. TA and the formula of C230H321N72O120P19S19. Thus, the expected molecular weight for ASO comprising SEQ ID NO: 284 is about 7212.3 Da. FIG. 1B shows the peak mass for ASO comprising SEQ ID NO: 284 as measured by LC-MS is 7214.3 Da. FIG. 1C shows the deconvolution peak report of LC-MS for ASO comprising SEQ ID NO: 284.

The antisense oligonucleotide comprising SEQ ID NO: 285 has the formula of C230 N69 O124 P19 S19 H318, with an estimated molecular weight of 7231.11 Da. FIG. 1D shows the peak mass for ASO comprising SEQ ID NO: 285 as measured by LC-MS is 7232.5. FIG. 1E shows the deconvolution peak report of LC-MS for ASO comprising SEQ ID NO: 285.

### Example 9: In Vivo Testing of Gapmers Targeting MAPT

Generation of Human Tau (hTau) Transgenic Mice

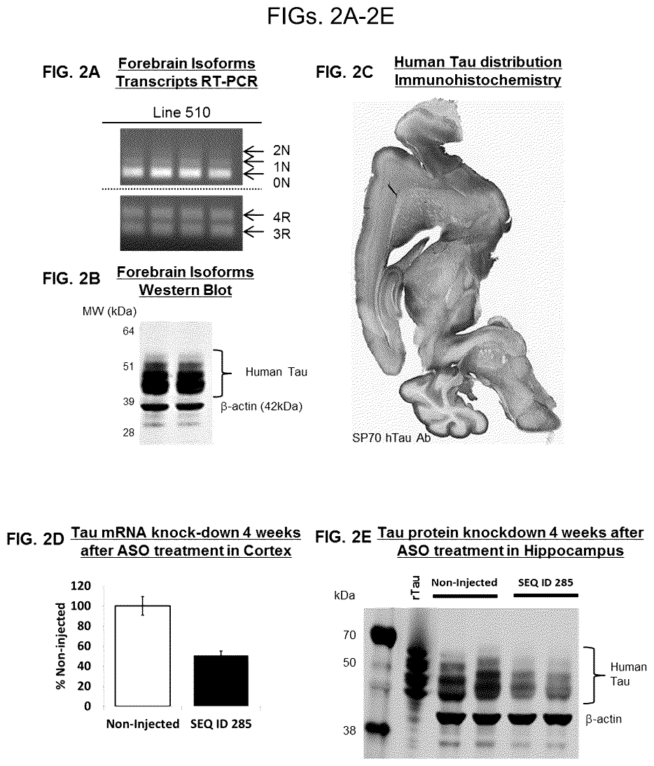

The BAC vectors (pBACe3.6) containing the human tau gene MAPT was obtained from Life technologies human genomic libraries. Three vectors predicted to contain all regulatory regions of MAPT gene were screened. Human genomic DNA were subjected to standard PCR to test for the presence of each exon, primers spanning introns of the human tau gene and regulatory regions were also used to determine the sequence of the clones. Comparison to human DNA showed that one clone (RP11 669E14) was intact for the all portions of the tau gene. This BAC vector was used to generate hTau BAC transgenic mice. The purified DNA was injected into fertilized embryos of C57BL/6 mice. Tail DNA from founder pups was digested with restriction enzymes and hybridized with exon specific probes, using similarly digested human DNA as a control to test for transgene integrity. Positive founder pups were expanded. Several hTau BAC transgenic lines were generated and one line showed human MAPT mRNA and protein expression (FIGS. 2A-2C). This line expressed all six human brain transcripts and protein isoforms found in the human brain (FIGS. 2A-2C). Heterozygote hTau BAC transgenic mice carry one copy of the transgene and the levels of RNA and human tau expression are comparable to the endogenous murine Tau expression.

### In Vivo Knockdown of Human Tau by Antisense Oligonucleotides

Selected antisense oligonucleotides were tested in vivo. Groups of five hTau BAC transgenic mice were administered with 1, 10, 50, 200, or 400 ug of a selected antisense oligonucleotide by intracerebroventricular (ICV) bolus injection, or left untreated as a group of control mice. All procedures were performed under isofluorane anesthesia and in accordance with IACUC regulations. For ICV bolus injections, the antisense oligonucleotide was injected into the right lateral ventricle of hTau BAC transgenic mice. Two or four microliters of a PBS solution containing 100 ug/ul of oligonucleotide were injected. Tissues were collected immediately after, 1 hour, 4 hours, 24 hours, 2 weeks, 4 weeks, 12 weeks or 24 weeks after oligonucleotide administration. RNA was extracted from hippocampus or cortex and examined for human tau mRNA expression by real-time PCR analysis. Human tau mRNA levels were measured as described above. Results were calculated as percent inhibition of human tau mRNA expression normalized to GAPDH levels compared to the control untreated mice. Protein was extracted from hippocampus or cortex and examined for human tau protein expression level by ELISA and normalized to total protein level.

The in vivo activity of 5-10-5 gapmers comprising SEQ ID NO: 284 or SEQ ID NO: 285 were tested using the methods described above. As shown in Table 12, both antisense oligonucleotides significantly inhibited human tau mRNA expression in cortex and hippocampus 2 weeks after a single ICV injection of antisense oligonucleotides. The knockdown of human tau mRNA was approximately 65% in both cortex and hippocampus for the gapmer comprising SEQ ID NO: 285. The knockdown of human tau mRNA was approximately 42% in both cortex and hippocampus for the gapmer comprising SEQ ID NO: 284. For tau protein level after 2 weeks of ASO treatment, the gapmer comprising SEQ ID NO: 285 knocked down about 50% of tau protein expression in cortex; and the gapmer comprising SEQ ID NO: 284 knocked down about 36% of the tau protein expression in cortex. We did not observe a significant reduction of tau protein level in hippocampus after 2 weeks of ASO treatment.

TABLE 12
Inhibition of Tau mRNA and protein expression by
5-10-5 MOE gapmers in vivo

| | % Residual | | % Residual | |
|---|---|---|---|---|
| | tau mRNA two weeks | | tau protein two weeks | |
| ASO SEQ | after ASO treatment¹² | | after ASO treatment¹³ | |
| ID NO | Cortex | Hippocampus | Cortex | Hippocampus |
| 285 | 35.26 | 33.55 | 50.8 | 91.1 |
| 284 | 58.8 | 56.77 | 64 | 100 |

¹²% Residual tau mRNA is the level of tau mRNA in the indicated brain tissue of hTau BAC transgenic mice two weeks after a single ICV injection of the indicated ASO, as compared to the level of tau mRNA in the corresponding brain tissue of the control htau BAC transgenic mice that were not treated with ASO.
¹³% Residual tau protein is the level of tau protein in the indicated brain tissue of hTau BAC transgenic mice two weeks after a single ICV injection of the indicated ASO, as compared to the level of tau protein in the corresponding brain tissue of the control hTau BAC transgenic mice that were not treated with ASO.

Tau mRNA and protein level were also tested 4 weeks after the single ICV injection of the gapmers. The antisense oligonucleotides comprising SEQ ID NO: 285 significantly inhibited human tau mRNA (FIG. 2D) and protein (FIG. 2E) expression in brain. The knockdown of human tau mRNA was approximately 60% in both cortex and hippocampus for the gapmer comprising SEQ ID NO: 285 (FIG. 2D and data unshown). Western blot analysis showed that the gapmer comprising SEQ ID NO: 285 knocked down human tau protein level by approximately 50% in the hippocampus 4 weeks post treatment (FIG. 2E).

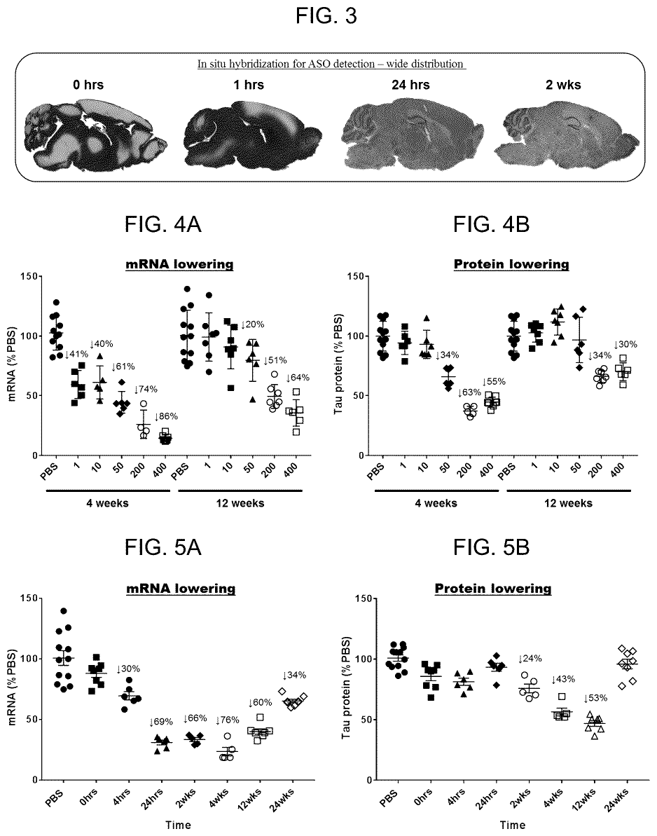

To detect the brain distribution of the antisense oligonucleotides in the brain of hTau BAC transgenic mice, in situ hybridization experiments were performed using a double digoxigenin (DIG) labeled Locked Nucleic Acid (LNA™, Exiqon) probe. Double-DIG LNA probes complementary to target antisense oligonucleotides were hybridized overnight. The probes were then detected using a sheep anti-DIG alkaline phosphatase conjugated antibody (Roche Diagnostics, Cat. #11093274910) and the colorimetric reaction of Nitro Blue Tetrazolium conjugated with the alkaline phosphatase substrate 5-Bromo-4-Chloro-3-Indolyl Phosphate (BCIP). The brain distribution of the antisense oligonucleotide of SEQ ID NO: 285 in a representative experiment was shown in FIG. 3, which shows initial diffusion of the ASO from the ventricles to the mouse brain parenchyma and the distribution signal does not change between 24 hrs and 2 weeks. (FIG. 3). The antisense oligonucleotide is stable in the brain even after 4 weeks (data unshown).

Dose-dependent inhibition of human tau mRNA (FIG. 4A) and protein (FIG. 4B) expression in hTau BAC transgenic mouse by the antisense oligonucleotide of SEQ ID NO: 285 was observed (FIGS. 4A and 4B).

The time course of human tau mRNA (FIG. 5A) and protein (FIG. 5B) expression level in hTau BAC transgenic mouse after a single ICV injection of 200 ug of the antisense oligonucleotide of SEQ ID NO: 285 showed sustained inhibition of tau mRNA and protein expression up to 12 weeks by the antisense oligonucleotide of SEQ ID NO: 285 (FIGS. 5A and 5B).

### Example 10: Inhibition of Human Tau Expression in Huh7 and SH-SY5Y Cells by Additional 5-10-5 Gapmers with 5-Methylcytosine

Additional gapmers sequences with 5-methylcytosine targeting tau were tested for inhibiting human Tau mRNA expression in vitro Huh7 and SH-SY5Y cells as described above. Results are presented as percent residual Tau mRNA relative to control cells treated with PBS. Table 13 shows the activities of additional screened sequences of 5-10-5 gapmers in Huh7 and SH-SY5Y cells.

TABLE 13
Inhibition of Tau mRNA by 5-10-5 MOE gapmers in Huh7 and SH-SY5Y cells

| | | % Residual | % Residual |
|---|---|---|---|
| SEQ | | mRNA in | mRNA in |
| ID | | SHSY5Y | HUH7 |
| NO | ASO Sequence¹⁴ | cells¹⁵ | cells¹⁶ |
| 307 | G*^{m}C*^{m}C*^{m}C*T*T^{m}CTGG^{m}C^{m}CTGGA*G*G*G*G* | 66.8 | 34.7 |
| 308 | T*G*G*^{m}C*^{m}C*CTT^{m}CTGG*^{m}C^{m}CTG*G*A*G*G* | 57.7 | 41.1 |
| 309 | G*^{m}C*T*G*G*TG^{m}CTT^{m}CAGGTT*^{m}C*T*^{m}C*A* | 60.4 | 42.7 |
| 310 | T*^{m}C*A*G*G*T^{m}CAA^{m}CTGGTTT*G*T*A*G* | 30.5 | 37.3 |
| 311 | T*G*^{m}C*T*^{m}C*AGGT^{m}CAA^{m}CTGG*T*T*T*G* | 42.6 | 35.5 |
| 312 | T*T*G*^{m}C*T*^{m}CAGGT^{m}CAA^{m}CTG*G*T*T*T* | 44.9 | 44.7 |
| 313 | ^{m}C*^{m}C*T*T*G*^{m}CT^{m}CAGGT^{m}CAA^{m}C*T*G*G*T | 10.3 | 20.9 |
| 314 | ^{m}C*^{m}C*^{m}C*T*^{m}C*TT^{m}CTA^{m}CATGGA*G*G*G*G* | 67.7 | 51.3 |
| 315 | T*T*^{m}C*T*^{m}C*^{m}C^{m}CT^{m}CTT^{m}CTA^{m}CA*T*G*G*A* | 76.7 | 95.1 |
| 316 | ^{m}C*T*T*^{m}C*T*^{m}C^{m}C^{m}CT^{m}CTT^{m}CTA^{m}C*A*T*G*G* | 68.2 | 77.6 |
| 317 | ^{m}C*^{m}C*T*T*^{m}C*T^{m}C^{m}C^{m}CT^{m}CTT^{m}CTA*^{m}C*A*T*G* | 50.4 | 72.3 |
| 318 | ^{m}C*A**A*A*T*^{m}C^{m}CTTTGTTG^{m}CT*G*^{m}C*^{m}C*A* | 33.6 | 54.4 |
| 319 | T*^{m}C*A*A*A*T^{m}C^{m}CTTTGTTG^{m}C*T*G*^{m}C*^{m}C* | 28.5 | 44.5 |
| 320 | T*G*G*^{m}C*T*^{m}C^{m}CA^{m}CGAA^{m}CA^{m}CA*^{m}C*^{m}C*A*A* | 24.2 | 55.1 |
| 321 | G*T*G*G*^{m}C*T^{m}C^{m}CA^{m}CGAA^{m}CA^{m}C*A*^{m}C*^{m}C*A* | 22.9 | 51.6 |
| 322 | T*G*T*G*G*^{m}CT^{m}C^{m}CA^{m}CGAA^{m}CA*^{m}C*A*^{m}C*^{m}C* | 25.4 | 62.4 |
| 323 | ^{m}C*T*G*T*G*G^{m}CT^{m}C^{m}CA^{m}CGAA^{m}C*A*^{m}C*A*^{m}C* | 31.1 | 49.2 |
| 324 | ^{m}C*^{m}C*T*G*T*GG^{m}CT^{m}CACA^{m}CGAA*^{m}C*A*^{m}C*A* | 34.8 | 56.7 |
| 325 | G*^{m}C*^{m}C*T*G*TGG^{m}CT^{m}C^{m}CA^{m}CGA*A*^{m}C*A*^{m}C* | 31.4 | 51.3 |
| 326 | T*G*^{m}C*^{m}C*T*GTGG^{m}CT^{m}C^{m}CA^{m}CG*A*A*^{m}C*A* | 24.2 | 50.2 |
| 327 | ^{m}C*T*G*^{m}C*^{m}C*TGTGG^{m}CT^{m}C^{m}CA^{m}C*G*A*A*^{m}C* | 18.9 | 56.7 |
| 328 | T*^{m}C*T*G*^{m}C*^{m}CTGTGG^{m}CT^{m}C^{m}CA*^{m}C*G*A*A* | 25.0 | 43.7 |
| 329 | G*T*^{m}C*T*G*^{m}C^{m}CTGTGG^{m}CT^{m}C^{m}C*A*^{m}C*G*A* | 10.1 | 35.5 |
| 330 | ^{m}C*G*T*^{m}C*T*G^{m}*C^{m}CTGTGG^{m}CT^{m}C*^{m}C*A*^{m}C*G* | 12.6 | 37.9 |
| 331 | T*^{m}C*G*T*^{m}C*TG^{m}C^{m}CTGTGG^{m}CT*^{m}C*^{m}C*A*^{m}*C* | 9.1 | 44.6 |
| 332 | A*T*^{m}C*G*T*^{m}CTG^{m}C^{m}CTGTGG^{m}C*T*^{m}C*^{m}C*A* | 18.8 | 55.4 |
| 333 | ^{m}C*A*T*^{m}C*G*T^{m}CTG^{m}C^{m}CTGTGG*^{m}*C*T*^{m}C*^{m}C* | 17.1 | 62.3 |
| 334 | A*^{m}C*A*T*^{m}C*GT^{m}CTG^{m}C^{m}CTGTG*G*^{m}C*T*^{m}C* | 14.3 | 57.7 |
| 335 | G*A*^{m}C*A*T*^{m}CGT^{m}CTG^{m}C^{m}CTGT*G*G*^{m}C*T* | 11.7 | 33.5 |
| 336 | T*G*A*^{m}C*A*T^{m}CGT^{m}CTG^{m}C^{m}CTG*T*G*G*^{m}C* | 15.2 | 38.4 |
| 337 | T*T*G*A*^{m}C*AT^{m}CGT^{m}CT^{m}C^{m}CT*G*T*G*G* | 26.2 | 43.3 |
| 338 | G*T*T*G*A*^{m}CAT^{m}CGT^{m}CTG^{m}C^{m}C*T*G*T*G* | 18.0 | 44.5 |
| 285 | G*G*T*T*G*A^{m}CAT^{m}CGT^{m}CTG^{m}C*^{m}C*T*G*T* | 17.2 | 42.7 |
| 339 | A*G*G*T*T*GA^{m}CAT^{m}CGT^{m}CTG*^{m}C*^{m}C*T*G* | 23.4 | 48.9 |
| 340 | A*A*G*G*T*TGA^{m}CAT^{m}CGT^{m}CT*G*^{m}C*^{m}C*T* | 17.2 | 38.6 |
| 341 | ^{m}C*A*A*G*G*TTGA^{m}CAT^{m}CGT^{m}C*T*G*^{m}C*^{m}C* | 25.7 | 47.4 |
| 342 | A*MC*A*A*G*GTTGA^{m}CAT^{m}CGT*^{m}C*T*G*^{m}C* | 24.3 | 43.0 |
| 343 | ^{m}C*A*^{m}C*A*A*GGTTGA^{m}CAT^{m}CG*T*^{m}C*T*G* | 28.9 | 52.9 |
| 344 | A*^{m}C*A*^{m}C*A*AGGTTGA^{m}CAT^{m}C*G*T*^{m}C*T* | 23.0 | 51.4 |
| 345 | ^{m}C*A*^{m}C*A*^{m}C*AAGGTTGA^{m}CAT*^{m}C*G*T*^{m}C* | 33.5 | 78.2 |
| 346 | T*^{m}C*A*^{m}C*A*^{m}CAAGGTTGA^{m}CA*T*^{m}C*G*T* | 44.4 | 60.4 |
| 347 | ^{m}C*T*^{m}C*A*^{m}C*A^{m}CAAGGTGA^{m}C*A*T*^{m}C*G* | 35.4 | 67.6 |
| 348 | A*^{m}C*T*^{m}C*A*^{m}CA^{m}CAAGGTGA*^{m}C*A*T*^{m}C* | 61.9 | 66.1 |
| 349 | ^{m}C*A*^{m}C*T*^{m}C*A^{m}CA^{m}CAAGGTTG*A*^{m}C*A*T* | 67.3 | 72.0 |
| 350 | A*^{m}C*A*^{m}C*T*^{m}CA^{m}CA^{m}CAAGGTT*G*A*^{m}C*A* | 64.0 | 72.7 |
| 351 | ^{m}C*A*^{m}C*A*^{m}C*T^{m}CA^{m}CA^{m}CAAGGT*T*G*A*^{m}C* | 46.3 | 64.8 |
| 352 | T*^{m}C*A*^{m}C*A*^{m}CT^{m}CA^{m}CA^{m}CA^{m}CAAGG*T*T*G*A* | 55.9 | 73.2 |
| 353 | G*T*^{m}C*A*^{m}C*A^{m}CT^{m}CA^{m}CA^{m}CAAG*G*T*T*G* | 31.9 | 53.3 |
| 354 | ^{m}C*G*T*^{m}C*A*^{m}CA^{m}CT^{m}CA^{m}CA^{m}CAA*G*G*T*T* | 29.1 | 57.6 |
| 355 | ^{m}C*^{m}C*G*T*^{m}C*A^{m}CA^{m}CT^{m}CA^{m}CA^{m}CA*A*G*G*T* | 33.5 | 57.7 |
| 356 | ^{m}C*^{m}C*^{m}C*^{m}C*G*T^{m}CA^{m}CA^{m}CT^{m}CA^{m}CA*^{m}C*A*A*G* | 33.3 | 70.1 |
| 357 | ^{m}C*^{m}C*^{m}C*T*T*^{m}CT^{m}C^{m}C^{m}CA^{m}CAGG^{m}C*T*G*^{m}C*^{m}C* | 40.3 | 65.4 |
| 358 | ^{m}C*A*T*^{m}C*A*AGGT^{m}CAGT^{m}CTT*T*T*MC*T* | 43.5 | 61.8 |
| 359 | ^{m}C*^{m}C*A*A*^{m}C*^{m}CTT^{m}CAGAA^{m}CT^{m}C*A*A*T*A* | 30.7 | 71.0 |
| 360 | T*^{m}C*^{m}C*A*A*^{m}C^{m}CTT^{m}CAGAA^{m}CT*^{m}C*A*A*T* | 30.7 | 77.6 |
| 361 | T*T*^{m}C*^{m}C*A*A^{m}C^{m}CTT^{m}CAGAA^{m}C*T*^{m}C*A*A* | 29.7 | 66.5 |
| 362 | G*T*T*^{m}C*^{m}C*AA^{m}C^{m}CTT^{m}CAGAA*^{m}C*T*^{m}C*A* | 22.9 | 72.7 |
| 363 | A*G*T*T*^{m}C*^{m}CAA^{m}C^{m}CTT^{m}CAGA*A*^{m}C*T*^{m}C* | 47.6 | 66.5 |
| 364 | ^{m}C*A*G*T*T*^{m}C^{m}CAA^{m}C^{m}CTT^{m}CAG*A*A*^{m}C*T* | 63.6 | 70.0 |
| 365 | G*^{m}C*A*G*T*T^{m}C^{m}CAA^{m}C^{m}CTT^{m}CA*G*A*A*^{m}C* | 28.6 | 47.6 |
| 366 | G*T*^{m}C*^{m}C*^{m}C*AGGT^{m}CTG^{m}CAAA*G*T*G*G* | 18.1 | 41.7 |
| 367 | A*A*G*T*^{m}C*^{m}C^{m}CAGGT^{m}CTG^{m}CA*A*A*G*T* | 48.5 | 68.1 |
| 368 | A*A*A*G*T*^{m}C^{m}C^{m}CAGGT^{m}CTG^{m}C*A*A*A*G* | 53.0 | 73.7 |
| 369 | G*G*^{m}C*A*^{m}C*AAGT^{m}C^{m}CTTA^{m}CA*A*A*G*A* | 43.7 | 80.9 |
| 370 | A*G*G*^{m}C*A*^{m}CAAGT^{m}C^{m}CTTA^{m}C*A*A*A*G* | 41.8 | 76.8 |
| 371 | T*^{m}C*A*^{m}C*^{m}C*^{m}CT^{m}CAGTATGGA*G*T*A*G* | 35.8 | 67.9 |
| 372 | T*T*^{m}C*A*^{m}C*^{m}C^{m}CT^{m}CAGTATGG*A*G*T*A* | 35.7 | 58.3 |
| 373 | T*T*T*^{m}C*A*^{m}C^{m}C^{m}CT^{m}CAGTATG*G*A*G*T* | 33.9 | 65.5 |
| 374 | A*T*T*T*^{m}C*A^{m}C^{m}C^{m}CT^{m}CAGTAT*G*G*A*G* | 51.1 | 54.0 |
| 375 | A*A*T*T*T*^{m}CA^{m}C^{m}C^{m}CT^{m}CAGTA*T*G*G*A* | 86.4 | 67.7 |
| 376 | ^{m}C*^{m}C*T*T*A*ATTT^{m}CA^{m}C^{m}C^{m}CT^{m}C*A*G*T*A* | 35.6 | 72.8 |
| 377 | ^{m}C*^{m}C*^{m}C*T*T*AATTT^{m}CA^{m}C^{m}C^{m}CT*^{m}C*A*G*T* | 32.4 | 60.4 |
| 378 | T*^{m}C*^{m}C*^{m}C*T*TAATTT^{m}CA^{m}C^{m}C^{m}C*T*^{m}C*A*G* | 26.7 | 71.7 |
| 379 | T*T*^{m}C*^{m}C*^{m}C*TTAATTT^{m}CA^{m}C^{m}C*^{m}C*T*^{m}C*A* | 20.4 | 73.9 |
| 380 | ^{m}C*T*T*^{m}C*^{m}C*^{m}CTTAATTT^{m}CA^{m}C*^{m}C*^{m}C*T*^{m}C* | 28.1 | 86.4 |
| 381 | A*^{m}C*T*^{m}C*T*TGTG^{m}C^{m}CTGGA^{m}C*T*T*T*G* | 32.4 | 44.6 |
| 382 | ^{m}C*A*^{m}C*T*^{m}C*TTGTG^{m}C^{m}CTGGA*^{m}C*T*T*T* | 33.4 | 58.2 |
| 383 | ^{m}C*^{m}C*A*^{m}C*T*^{m}CTTGTG^{m}C^{m}CTGG*A*^{m}C*T*T* | 26.8 | 68.5 |
| 384 | ^{m}C*^{m}C*^{m}C*A*^{m}C*T^{m}CTTGTG^{m}C^{m}CTG*G*A*^{m}C*T* | 16.5 | 43.4 |
| 385 | T*^{m}C*^{m}C*^{m}C*A*^{m}CT^{m}CTTGTG^{m}C^{m}CT*G*G*A*^{m}C* | 10.6 | 38.9 |
| 386 | G*T*^{m}C*^{m}C*^{m}C*A*^{m}CT^{m}CTTGTG^{m}C^{m}C*T*G*G*A* | 23.0 | 37.4 |
| 387 | G*G*T*^{m}C*^{m}C*^{m}CA^{m}CT^{m}CTTGTG^{m}C*^{m}C*T*G*G* | 31.0 | 36.9 |
| 388 | G*G*G*T*^{m}C*^{m}C^{m}CA^{m}CT^{m}CTTGTG*^{m}C*^{m}C*T*G* | 45.9 | 47.5 |
| 389 | G*T*G*^{m}C*^{m}C*^{m}CTGG^{m}CT^{m}CA^{m}CAT*^{m}C*T*G*T* | 42.8 | 81.7 |
| 390 | A*G*T*G*^{m}C*^{m}C^{m}CTGG^{m}CT^{m}CA^{m}CA*T*^{m}C*T*G* | 28.1 | 51.0 |
| 391 | ^{m}C*A*G*T*G*^{m}C^{m}C^{m}CTGG^{m}CT^{m}CA^{m}C*A*T*^{m}C*T* | 49.1 | 85.8 |
| 392 | G*^{m}C*A*G*T*G^{m}C^{m}C^{m}CTGG^{m}CT^{m}CA*^{m}C*A*T*^{m}C* | 34.4 | 65.8 |
| 393 | A*G*^{m}C*A*G*TG^{m}C^{m}C^{m}CTGG^{m}CT^{m}C*A*^{m}C*A*T* | 40.5 | 72.6 |
| 394 | T*G*A*G*^{m}C*AGTG^{m}C^{m}C^{m}CTGG^{m}C*T*^{m}C*A*^{m}C* | 91.8 | 57.9 |
| 395 | G*^{m}C*A*T*G*G^{m}CTT^{m}C^{m}CAG^{m}CTG*G*G*G*A*^{m}C* | 38.5 | 60.3 |
| 396 | A*G*^{m}C*T*G**^{m}CT^{m}C^{m}CAG^{m}CAGAA*^{m}C*A*G*A* | 80.8 | 78.5 |
| 397 | T*A*T*A*T*GTT^{m}CAG^{m}CTG^{m}CT*^{m}C*^{m}C*A*G* | 49.9 | 63.2 |
| 398 | G*T*A*T*A*TGTT^{m}CAG^{m}CTG^{m}C*T*^{m}C*^{m}C*A* | 55.2 | 64.3 |
| 399 | T*G*T*A*T*ATGG^{m}CAG^{m}CTG*^{m}C*T*^{m}C*^{m}C* | 59.9 | 74.6 |
| 400 | G*^{m}C*A*G*G*G^{m}CAA^{m}CAT^{m}CTAT*G*T*A*T* | 58.3 | 73.2 |
| 401 | G*G*^{m}C*A*G*GG^{m}CAA^{m}CAT^{m}CTA*T*G*T*A* | 61.5 | 74.5 |
| 402 | G*G*G*^{m}C*A*GGG^{m}CAA^{m}CAT^{m}CT*A*T*G*T* | 48.1 | 68.0 |
| 403 | T*^{m}C*A*^{m}C*T*^{m}CTGGTGAAT^{m}C^{m}C*A*A*G*^{m}C* | 21.9 | 54.5 |
| 404 | G*T*^{m}C*A*^{m}C*T^{m}CTGGTGAAT^{m}C*^{m}C*A*A*G* | 13.8 | 61.3 |
| 405 | A*G*T*^{m}C*A*^{m}CT^{m}CTGGTGAAT*^{m}C*^{m}C*A*A* | 15.1 | 55.2 |
| 406 | T*A*G*T*^{m}C*A^{m}CT^{m}CTGGTGAA*T*^{m}C*^{m}C*A* | 36.7 | 70.9 |
| 407 | A*T*A*G*T*^{m}CA^{m}CT^{m}CTGGTGA*A*T*^{m}C*^{m}C* | 42.4 | 76.6 |
| 408 | ^{m}C*A*T*A*G*T^{m}CA^{m}CT^{m}CTGGTG*A*A*T*^{m}C* | 57.0 | 77.2 |
| 409 | T*^{m}C*A*T*A*GT^{m}CA^{m}CT^{m}CTGGT*G*A*A*T* | 45.8 | 65.5 |
| 410 | ^{m}C*T*G*G*T*^{m}C^{m}CTGTTGGGT*^{m}C*^{m}C*^{m}C*A* | 37.2 | 60.5 |
| 411 | A*T*^{m}C*^{m}C*T*GTG^{m}CTT^{m}CAGG^{m}C*^{m}C*T*T*^{m}C* | 31.2 | 66.6 |
| 412 | A*A*T*^{m}C*^{m}C*TGTG^{m}CTT^{m}CAGG*^{m}C*^{m}C*T*T* | 41.2 | 73.5 |
| 413 | ^{m}C*T*A*A*T*^{m}C^{m}CTGTG^{m}CTT^{m}CA*G*G*^{m}C*^{m}C* | 38.7 | 65.1 |
| 414 | ^{m}C*^{m}C*T*A*A*T^{m}C^{m}CTGTG^{m}CTT^{m}C*A*G*G*^{m}C* | 31.9 | 64.8 |
| 415 | T*^{m}C*^{m}C*T*A*AT^{m}C^{m}CTGTG^{m}CTT*^{m}C*A*G*G* | 45.9 | 73.6 |
| 416 | G*T*^{m}C*^{m}C*T*AAT^{m}C^{m}CTGTG^{m}CT*T*^{m}C*A*G* | 50.0 | 80.9 |
| 417 | A*G*T*^{m}C*^{m}C*TAAT^{m}C^{m}CTGTG^{m}C*T*T*^{m}C*A* | 51.9 | 77.1 |
| 418 | ^{m}C*A*G*T*^{m}C*^{m}CTAAT^{m}C^{m}CTGTG*^{m}C*T*T*^{m}C* | 53.2 | 68.4 |
| 419 | T*^{m}C*A*G*T*^{m}C^{m}CTAAT^{m}C^{m}CTGT*G*^{m}C*T*T* | 58.9 | 78.3 |
| 420 | T*T*^{m}C*A*G*T^{m}C^{m}CTAAT^{m}C^{m}CTG*T*G*^{m}C*T* | 51.1 | 72.9 |
| 421 | ^{m}C*T*T*^{m}C*A*GT^{m}C^{m}CTAAT^{m}C^{m}CT*G*T*G*^{m}C* | 49.4 | 69.1 |
| 422 | G*^{m}C*T*T*^{m}C*AGT^{m}C^{m}CTAAT^{m}C^{m}C*T*G*T*G* | 39.5 | 56.8 |
| 423 | G*G*A*G*T*TGTAAG^{m}C^{m}CT^{m}C^{m}C*T*T*T*G* | 61.8 | 66.4 |
| 424 | G*^{m}C*T*^{m}C*T*GGT^{m}CAAGG^{m}CTT*T*G*G*G* | 33.8 | 49.8 |
| 425 | T*G*^{m}C*T*^{m}C*TGGT^{m}CAAGG^{m}CT*T*T*G*G* | 37.9 | 55.5 |
| 426 | G*T*G*^{m}C*T*^{m}CTGGT^{m}CAAGG^{m}C*T*T*T*G* | 48.0 | 74.1 |
| 427 | G*G*T*G*^{m}C*T^{m}CTGGT^{m}CAAGG*^{m}C*T*T*T* | 51.4 | 68.7 |
| 428 | T*G*A*G*G*TG^{m}CT^{m}CTGGT^{m}CA*A*G*G*^{m}C* | 38.0 | 72.9 |
| 429 | T*T*T*^{m}C*T*^{m}CATGG^{m}CAG^{m}CAG*A*T*G*G* | 87.5 | 84.7 |
| 430 | T*G*^{m}C*T*G*AGTTT^{m}CTTTAG*G*^{m}C*A*G* | 61.8 | 94.7 |
| 431 | ^{m}C*T*G*^{m}C*T*GAGTTT^{m}CTTTA*G*G*^{m}C*A* | 51.5 | 61.1 |
| 432 | G*^{m}C*T*G*^{m}C*TGAGTTT^{m}CTTT*A*G*G*^{m}C* | 46.5 | 94.4 |
| 433 | G*G*^{m}C*T*G*^{m}CTGAGTTT^{m}CTT*T*A*G*G* | 61.8 | 80.4 |
| 434 | A*G*G*^{m}C*T*G^{m}CTGAGTTT^{m}CT*T*T*A*G* | 68.1 | 74.7 |
| 435 | G*A*G*G*^{m}C*TG^{m}CTGAGTTT^{m}C*T*T*T*A* | 52.3 | 83.7 |
| 436 | T*G*A*G*G*^{m}CTG^{m}CTGAGTTT*^{m}C*T*T*T* | 62.3 | 71.5 |
| 437 | ^{m}C*T*G*^{m}C*^{m}C*AAGT^{m}C^{m}C^{m}CT^{m}CAG*G*G*T*T* | 43.3 | 74.6 |
| 438 | A*^{m}C*T*G*^{m}C*^{m}CAAGT^{m}C^{m}C^{m}CT^{m}CA*G*G*G*T* | 53.4 | 78.8 |
| 439 | T*A*^{m}C*T*G*^{m}C^{m}CAAGT^{m}C^{m}C^{m}CT^{m}C*A*G*G*G* | 34.5 | 74.9 |
| 440 | ^{m}C*T*A*^{m}C*T*G^{m}C^{m}CAAGT^{m}C^{m}C^{m}CT*^{m}C*A*G*G* | 35.4 | 73.6 |
| 441 | T*^{m}C*T*A*^{m}C*TG^{m}C^{m}CAAGT^{m}C^{m}C^{m}C*T*^{m}C*A*G* | 65.7 | 84.0 |
| 442 | T*T*^{m}C*T*A*^{m}CTG^{m}C^{m}CAAGT^{m}C^{m}C*^{m}C*T*^{m}C*A* | 47.9 | 93.1 |
| 443 | T*T*T*^{m}C*T*A^{m}CTG^{m}C^{m}CAAGT^{m}C*^{m}C*^{m}C*T*^{m}C* | 53.6 | 85.3 |
| 444 | A*T*T*T*^{m}C*TA^{m}CTG^{m}C^{m}CAAGT*^{m}C*^{m}C*^{m}C*T* | 72.4 | 100.6 |
| 445 | G*A*T*T*T*^{m}CTA^{m}CTG^{m}C^{m}CAAG*T*^{m}C*^{m}C*^{m}C* | 68.6 | 79.7 |
| 446 | G*G*A*T*T*T^{m}CTA^{m}CTG^{m}C^{m}CAA*G*T*^{m}C*^{m}C* | 72.1 | 88.6 |
| 447 | T*G*G*A*T*TT^{m}CTA^{m}CTG^{m}C^{m}CA*A*G*T*^{m}C* | 65.7 | 79.7 |
| 448 | ^{m}C*T*G*G*A*TTT^{m}CTA^{m}CTG^{m}C^{m}C*A*A*G*T* | 51.0 | 68.6 |
| 449 | A*T*^{m}C*T*T*AGG^{m}CTGG^{m}C^{m}C^{m}C^{m}C*A*A*G*A* | 43.6 | 74.1 |
| 450 | T*G*A*T*^{m}C*TTAGG^{m}CTGG^{m}C^{m}C*^{m}C*^{m}C*A*A* | 38.8 | 70.0 |
| 451 | T*T*T*A*T*^{m}CTG^{m}C^{m}CAG^{m}CA^{m}CT*G*A*T*^{m}C* | 51.0 | 76.2 |
| 452 | A*T*T*T*A*T^{m}CTG^{m}C^{m}CAG^{m}CA^{m}C*T*G*A*T* | 55.2 | 77.7 |
| 453 | A*A*T*T*T*AT^{m}CTG^{m}C^{m}CAG^{m}CA*^{m}C*T*G*A* | 68.1 | 71.1 |
| 454 | T*A*T*A*T*^{m}C^{m}CTAT^{m}CTAG^{m}C^{m}C*^{m}C*A*^{m}C*^{m}C* | 60.1 | 88.2 |
| 455 | G*T*A*T*A*T^{m}C^{m}CTAT^{m}CTAG^{m}C*^{m}C*^{m}C*A*^{m}C* | 64.2 | 85.2 |
| 456 | A*G*T*A*T*AT^{m}C^{m}CTAT^{m}CTAG*^{m}C*^{m}C*^{m}C*A* | 62.8 | 86.2 |
| 457 | A*A*^{m}C*^{m}C*^{m}C*^{m}CAAGGG^{m}C^{m}CT^{m}CT*A*A*^{m}C*T* | 83.3 | 90.7 |
| 458 | G*^{m}C*A*A*^{m}C*^{m}CAGATGT^{m}C^{m}CAT*A*T*T*^{m}C* | 50.9 | 87.2 |
| 459 | G*G*^{m}C*T*T*AGGA^{m}C^{m}C^{m}C^{m}CTGA*A*A*G*A* | 59.8 | 71.1 |
| 460 | G*G*^{m}C*A*T*GATTGTGGG^{m}CT*T*A*G*G* | 32.3 | 51.6 |
| 461 | A*G*G*^{m}C*A*TGATTGTGGG^{m}C*T*T*A*G* | 31.0 | 60.2 |
| 462 | G*T*A*A*^{m}C*^{m}C^{m}CTTTT^{m}CAAAG*^{m}C*T*G*A* | 50.2 | 63.6 |
| 463 | G*G*T*A*A*^{m}C^{m}C^{m}CTTTT^{m}CAAA*G*^{m}C*T*G* | 27.1 | 51.9 |
| 464 | G*G*G*T*A*A*^{m}C^{m}C^{m}CTTTT^{m}CAA*A*G*^{m}C*T* | 45.6 | 64.5 |
| 465 | A*G*G*G*T*AA^{m}C^{m}C^{m}CTTTT^{m}CA*A*A*G*^{m}C*C* | 60.3 | 61.5 |
| 466 | ^{m}C*A*G*G*G*TAA^{m}C^{m}C^{m}CTTTTMC*A*A*A*G* | 59.2 | 82.8 |
| 467 | ^{m}C*^{m}C*A*G*G*GTAA^{m}C^{m}C^{m}CTTTT*^{m}C*A*A*A* | 48.5 | 57.9 |
| 468 | ^{m}C*^{m}C*^{m}C*A*G*GGTAA^{m}C^{m}C^{m}CTTT*T*^{m}C*A*A* | 37.8 | 70.2 |
| 469 | G*^{m}C*^{m}C*^{m}C*A*GGGTAA^{m}C^{m}C^{m}CTT*T*T*^{m}C*A* | 31.9 | 58.2 |
| 470 | T*G*^{m}C*T*^{m}C*AA^{m}CATGG^{m}CAAA*^{m}C*T*^{m}C*A* | 42.1 | 70.5 |
| 471 | T*^{m}C*^{m}C*T*G*^{m}CTCAA^{m}CATGG^{m}C*A*A*A*^{m}C* | 45.2 | 77.7 |
| 472 | G*T*^{m}C*^{m}C*T*G^{m}CT^{m}CAA^{m}CATGG*^{m}C*A*A*A* | 42.9 | 64.0 |

¹⁴The nucleotides with * have a 2′-O-MOE modifications; the nucleotides without * are 2′-deoxynucleosides; and ^{m}C stands for 5-methylcytosine. The internucleoside linkages are phosphorothioate.
¹⁵% Residual mRNA is the level of tau mRNA in the SH-SY5Y cells treated with a single does of 2,000 nM of tau ASO for 24 hours as compared to the level of tau mRNA in controls cells treated with PBS.
¹⁶% Residual mRNA is the level of tau mRNA in the Huh7 cells treated with a single does of 25 nM of tau ASO for 48 hours as compared to the level of tau mRNA in control cells treated with PBS.

Gapmers that significantly decreased Tau mRNA expression in Table 13 were selected and tested in SH-SY5Y cells. The IC50 values of selected 5-10-5 gapmers with 5-methylcytosines were determined in SH-SY5Y cells as described above, and shown in Table 14.

TABLE 14
IC50 of selected 5-10-5 gapmers with
5-methylcytosine in SH-SY5Y

| SEQ | | |
|---|---|---|
| ID | | IC50 |
| NO | ASO Sequence¹⁷ | (nM) |
| 313 | ^{m}C*^{m}C*T*T*G*^{m}CT^{m}CAGGT^{m} | 1115 |
| | CAA^{m}C*T*G*G*T* | |
| 327 | ^{m}C*T*G*^{m}C*^{m}C*TGTGG^{m}CT^{m} | 844 |
| | C^{m}CA^{m}C*G*A*A*^{m}C* | |
| 329 | G*T*^{m}C*T*G*^{m}C^{m}CTGTGG^{m} | 481 |
| | CT^{m}C^{m}C*A*^{m}C*G*A* | |
| 330 | ^{m}C*G*T*^{m}C*T*G^{m}C^{m}CTGTG | 555 |
| | G^{m}CT^{m}C*^{m}C*A*^{m}C*G* | |
| 331 | T*^{m}C*G*T*^{m}C*TG^{m}C^{m}CTGT | 818 |
| | GG^{m}CT*^{m}C*^{m}C*A*^{m}C* | |
| 332 | A*T*^{m}C*G*T*^{m}CTG^{m}C^{m}CTG | 918 |
| | TGG^{m}C*T*^{m}C*^{m}C*A* | |
| 333 | ^{m}C*A*T*^{m}C*G*T^{m}CTG^{m}C^{m}C | 981 |
| | TGTGG*^{m}C*T*^{m}C*^{m}C* | |
| 334 | A*^{m}C*A*T*^{m}C*GT^{m}CTG^{m}C^{m} | 608 |
| | CTGTG*G*^{m}C*T*^{m}C* | |
| 335 | G*A*^{m}C*A*T*^{m}CGT^{m}CTG^{m} | 414 |
| | C^{m}CTGT*G*G*^{m}C*T* | |
| 336 | T*G*A*^{m}C*A*T^{m}CGT^{m}CT | 393 |
| | GMC^{m}CTG*T*G*G*^{m}C* | |
| 338 | G*T*T*G*A*^{m}CAT^{m}CGT^{m} | 588 |
| | CTG^{m}C^{m}C*T*G*T*G* | |
| 340 | A*A*G*G*T*TGA^{m}CAT^{m} | 496 |
| | CGT^{m}CT*G*^{m}C*^{m}C*T* | |
| 366 | G*T*^{m}C*^{m}C*^{m}C*AGGT^{m}C | 793 |
| | TG^{m}CAAA*G*T*G*G* | |
| 384 | ^{m}C*^{m}C*^{m}C*A*^{m}C*T^{m}CTT | 810 |
| | GTG^{m}C^{m}CTG*G*A*^{m}C*T* | |
| 385 | T*^{m}C*^{m}C*^{m}C*A*^{m}CT^{m}CTT | 954 |
| | GTG^{m}C^{m}CT*G*G*A*^{m}C* | |
| 404 | G*T*^{m}C*A*^{m}C*T^{m}CTGGT | 12035 |
| | GAAT^{m}C*^{m}C*A*A*G* | |
| 405 | A*G*T*^{m}C*A*^{m}CT^{m}CTGG | 743 |
| | TGAAT*^{m}C*^{m}CA*A* | |
| 381 | A*^{m}C*T*^{m}C*T*TGTG^{m}C^{m} | 2737 |
| | CTGGA^{m}C*T*T*T*G* | |

¹⁷The nucleotides with * have a 2′-O-MOE modification; the nucleotides without * are 2′-deoxynucleosides; and ^{m}C stands for 5-methylcytosine. The internucleoside linkages are phosphorothioate.

### Example 11: Inhibition of Monkey and Human Tau Expression by Antisense Oligonucleotides with 5-Methylcytosine

Some gapmers that significantly decreased Tau mRNA expression were selected and tested in COS1 green monkey cells. Results are presented as percent residual Tau mRNA relative to control cells treated with PBS. Table 15 shows the activities of selected 5-10-5 gapmers in COS1 cells.

TABLE 15
Inhibition of green monkey Tau mRNA by
5-10-5 MOE gapmers in Cos1 cells

| | | % Res- |
|---|---|---|
| SEQ | | idual |
| ID | | tau |
| NO | ASO Sequence¹⁸ | mRNA¹⁹ |
| 285 | G*G*T*T*G*A^{m}CAT^{m}CGT^{m} | 39 |
| | CTG^{m}C*^{m}C*T*G*T* | |
| 284 | ^{m}C*^{m}C*G*T*A*^{m}CGT^{m}C^{m}C^{m} | 61 |
| | CAG^{m}CGT*G*A*T*^{m}C* | |
| 473 | ^{m}C*^{m}C*^{m}C*G*T*A^{m}CGT^{m}C^{m} | 62 |
| | C^{m}CAG^{m}CG*T*G*A*T* | |
| 474 | G*G*^{m}C*^{m}C*A*G^{m}CGT^{m}C^{m} | 64 |
| | CGTGT^{m}C*A*^{m}C*^{m}C*^{m}C* | |
| 386 | G*T*^{m}C*^{m}C*^{m}C*A^{m}CT^{m}CT | 53 |
| | TGTG^{m}C^{m}C*T*G*G*A* | |
| 315 | G*A*^{m}C*A*T*^{m}CGT^{m}CTG | 39 |
| | ^{m}C^{m}CTGT*G*G*^{m}C*T* | |
| 384 | ^{m}C*^{m}C*^{m}C*A*^{m}C*T^{m}CTTG | 51 |
| | TG^{m}C^{m}CTG*G*A*^{m}C*T* | |
| 313 | ^{m}C*^{m}C*T*T*G*^{m}CT^{m}CAGG | 37 |
| | T^{m}CAA^{m}C*T*G*G*T* | |
| 366 | G*T*^{m}C*^{m}C*^{m}C*AGGT^{m}CT | 35 |
| | G^{m}CAAA*G*T*G*G* | |
| 329 | A*G*T*^{m}C*A*^{m}CT^{m}CTGGT | 34 |
| | GAAT*^{m}C*^{m}C*A*A* | |
| 405 | A*G*T*^{m}C*A*^{m}CT^{m}CTGGT | 37 |
| | GAAT*^{m}C*^{m}C*A*A* | |

¹⁸The nucleosides with * have a 2′-O-MOE modification; the nucleosides without * are 2′-deoxynucleosides; and ^{m}C stands for 5-methylcytosine. The internucleoside linkages are phosphorothioate.
¹⁹% Residual mRNA is the level of tau mRNA in the Cos1 cells treated with a single dose of 2,000 nM of tau ASP for 24 hours as compared to the level of tau mRNA in control cells treated with PBS.

Some antisense oligonucleotides that significantly decreased Tau mRNA expression were selected and tested in human embryonic stem cell (hESC) derived neurons. Results are presented as percent residual Tau mRNA relative to control cells treated with PBS. Table 16 shows the activities of selected antisense oligonucleotides in human neurons.

TABLE 16
Inhibition of human Tau expression hESC
derived neurons by selected Tau ASO

| SEQ | | % Res- |
|---|---|---|
| ID | | idual |
| NO | ASO Sequence²⁰ | mRNA²¹ |
| 285 | G*G*T*T*G*A^{m}CAT^{m}CGT^{m} | 9.8 |
| | CTG^{m}C*^{m}C*^{m}C*T*G*T* | |
| 475 | ^{m}C*^{m}C*A*T*G*^{m}C*G*A*G | 20.9 |
| | *^{m}C*T*G*A*T*A*A*A* | |
| 476 | G*^{m}C*A*T*^{m}C*G*T*^{m}C*A | 40.0 |
| | *G*^{m}C*T*T*A*^{m}C*^{m}C*T* | |
| 477 | ^{m}C*T*T*T*G*^{m}C*T*T*T | 16.2 |
| | *T*A*^{m}C*T*G*A*^{m}C*^{m}C* | |
| 478 | T*^{m}C*A*A*^{m}C*T*G*G*T* | 34.9 |
| | T*T*G*T*A*G*A*^{m}C* | |

²⁰The nucleotides with * have a 2′-O-MOE modification; the nucleotides without * are 2′-deoxynucleosides; and ^{m}C stands for 5-methylcytosine. The internucleoside linkages are phosphorothioate.
²¹%Residual mRNA is the level of tau mRNA in the hESC-deilved neurons treated with a single dose of 10 μM of tau ASO for 10 to 14 days as compared to the level of tau mRNA in control cells treated with PBS.

### Example 12: In Vivo Testing of Gapmers Targeting MAPT

The in vivo activity of selected 5-10-5 gapmers were tested using the methods described in Example 9. As shown in Table 17, some antisense oligonucleotides significantly inhibited human tau mRNA and protein expression in cortex and hippocampus.

TABLE 17
Inhibition of Tau mRNA and protein expression
by 5-10-5 MOE gapmers in vivo

| | | | % Residual | | % Residual | |
|---|---|---|---|---|---|---|
| | | Treat- | tau mRNA after | | tau protein after | |
| SEQ | | ment | ASO treatment²² | | ASO treatment²³ | |
| ID | Dose | Duration | Cor- | Hippo- | Cor- | Hippo- |
| NO | (ug) | (weeks) | tex | campus | tex | campus |
| 284 | 200 | 4 | 56 | 61 | 98 | 82 |
| 473 | 200 | 4 | 76 | 44 | 73 | 69 |
| 474 | 200 | 4 | 48 | 26 | 73 | 66 |
| 386 | 200 | 4 | 50 | 48 | 69 | 68 |
| 335 | 50 | 4 | N/T²⁴ | 76 | N/T | 103 |
| 384 | 50 | 4 | N/T | 65 | N/T | 111 |
| 313 | 50 | 4 | N/T | 98 | N/T | 144 |

²²% Residual tau mRNA is the level of tau mRNA in the indicated brain tissue of hTau BAC transgenic mice four weeks after a single ICV injection of the indicated ASO, as compared to the level of tau mRNA in the corresponding brain tissue of the control hTau BAC transgenic mice that were not treated with ASO.
²³% Residual tau protein is the level of tau protein in the indicated brain tissue of hTau BAC tnansgenic mice four weeks after a single ICV injection of the indicated ASO, as compared to the level of tau protein in the corresponding brain tissue of the control hTau BAC transgenic mice that were not treated with ASO.
²⁴N/T means not tested.

Unless defined otherwise, the technical and scientific terms used herein have the same meaning as they usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

1. An antisense oligonucleotide comprising a nucleotide sequence of
(SEQ ID NO: 285)
5′-G*G*T*T*G*A^{m}CAT^{m}CGT^{m}CTG^{m}C*^{m}C*T*G*T*-3′,
wherein:
each nucleotide with a * to the right of the nucleotide comprises a 2′-O-methoxyethyl modification of the 2′ position of a furanose ring, each nucleotide without a * to the right of the nucleotide comprises a 2′-deoxynucleoside, ^{m}C comprises 5-methylcytosine, and all internucleoside linkages are phosphorothioate.

2. A antisense oligonucleotide consisting of a nucleotide sequence of
(SEQ ID NO: 285)
5′-G*G*T*T*G*A^{m}CAT^{m}CGT^{m}CTG^{m}C*^{m}C*T*G*T*-3′,
wherein:
each nucleotide with a * to the right of the nucleotide comprises a 2′-O-methoxyethyl modification of the 2′ position of a furanose ring, each nucleotide without a * to the right of the nucleotide comprises a 2′-deoxynucleoside, ^{m}C comprises 5-methylcytosine, and all internucleoside linkages are phosphorothioate.

3. A pharmaceutical composition comprising:
an antisense oligonucleotide comprising a nucleotide sequence of:
(SEQ ID NO: 285)
5′-G*G*T*T*G*A^{m}CAT^{m}CGT^{m}CTG^{m}C*^{m}C*T*G*T*-3′,
wherein each nucleotide with a * to the right of the nucleotide comprises a 2′-O-methoxyethyl modification of the 2′ position of a furanose ring, each nucleotide without a * to the right of the nucleotide comprises a 2′-deoxynucleoside, ^{m}C comprises 5-methylcytosine, and all internucleoside linkages are phosphorothioate; and
a pharmaceutically acceptable carrier.

4. A pharmaceutical composition comprising:
an antisense oligonucleotide consisting of a nucleotide sequence of
(SEQ ID NO: 285)
5′-G*G*T*T*G*A^{m}CAT^{m}CGT^{m}CTG^{m}C*^{m}C*T*G*T*-3′,
wherein each nucleotide with a * to the right of the nucleotide comprises a 2′-O-methoxyethyl modification of the 2′ position of a furanose ring, each nucleotide without a * to the right of the nucleotide comprises a 2′-deoxynucleoside, ^{m}C comprises 5-methylcytosine, and all internucleoside linkages are phosphorothioate; and
a pharmaceutically acceptable carrier.

5. A method of treating a tau-associated disease in a subject in need thereof comprising administering to the subject the antisense oligonucleotide according to claim 1.

6. A method of treating a disease in a subject in need thereof, the method comprising administering to the subject the antisense oligonucleotide according to claim 1, wherein the disease is Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, or tuberous sclerosis.

7. A method of treating progressive supranuclear palsy (PSP) in a subject in need thereof, the method comprising administering to the subject the antisense oligonucleotide according to claim 1.

8. A method of treating Alzheimer's disease in a subject in need thereof, the method comprising administering to the subject the antisense oligonucleotide according to claim 1.

9. A method of treating frontotemporal dementia (FTD) in a subject in need thereof, the method comprising administering to the subject the antisense oligonucleotide according to claim 1.

10. A method of treating corticobasal degeneration (CBD) in a subject in need thereof, the method comprising administering to the subject the antisense oligonucleotide according to claim 1.

11. A method of treating a tau-associated disease in a subject in need thereof, the method comprising administering to the subject the antisense oligonucleotide according to claim 2.

12. A method of treating a disease in a subject in need thereof, the method comprising administering to the subject the antisense oligonucleotide according to claim 2, wherein the disease is Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, or tuberous sclerosis.

13. A method of treating progressive supranuclear palsy (PSP) in a subject in need thereof, the method comprising administering to the subject the antisense oligonucleotide according to claim 2.

14. A method of treating Alzheimer's disease in a subject in need thereof, the method comprising administering to the subject the antisense oligonucleotide according to claim 2.

15. A method of treating frontotemporal dementia (FTD) in a subject in need thereof, the method comprising administering to the subject the antisense oligonucleotide according to claim 2.

16. A method of treating corticobasal degeneration (CBD) in a subject in need thereof, the method comprising administering to the subject the antisense oligonucleotide according to claim 2.

17. A method of treating a tau-associated disease in a subject in need thereof, the method comprising administering to the subject the pharmaceutical composition according to claim 3.

18. A method of treating a disease in a subject in need thereof, the method comprising administering to the subject the pharmaceutical composition according to claim 3, wherein the disease is Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, or tuberous sclerosis.

19. A method of treating progressive supranuclear palsy (PSP) in a subject in need thereof, the method comprising administering to the subject the pharmaceutical composition according to claim 3.

20. A method of treating Alzheimer's disease in a subject in need thereof, the method comprising administering to the subject the pharmaceutical composition according to claim 3.

21. A method of treating frontotemporal dementia (FTD) in a subject in need thereof, the method comprising administering to the subject the pharmaceutical composition according to claim 3.

22. A method of treating corticobasal degeneration (CBD) in a subject in need thereof, the method comprising administering to the subject the pharmaceutical composition according to claim 3.

23. A method of treating a tau-associated disease in a subject in need thereof, the method comprising administering to the subject the pharmaceutical composition according to claim 4.

24. A method of treating a disease in a subject in need thereof, the method comprising administering to the subject the pharmaceutical composition according to claim 4, wherein the disease is Alzheimer's disease (AD), amyotrophic lateral sclerosis/parkinsonism-dementia complex (ALS-PDC), argyrophilic grain dementia (AGD), British type amyloid angiopathy, cerebral amyloid angiopathy, chronic traumatic encephalopathy (CTE), corticobasal degeneration (CBD), Creutzfeldt-Jakob disease (CJD), dementia pugilistica, diffuse neurofibrillary tangles with calcification, Down's syndrome, Dravet's Syndrome, epilepsy, frontotemporal dementia (FTD), frontotemporal dementia with parkinsonism linked to chromosome 17 (FTDP-17), frontotemporal lobar degeneration, ganglioglioma, gangliocytoma, Gerstmann-Straussler-Scheinker disease, Hallervorden-Spatz disease, Huntington's disease, inclusion body myositis, lead encephalopathy, Lytico-Bodig disease, meningioangiomatosis, multiple system atrophy, myotonic dystrophy, Niemann-Pick disease type C (NP-C), non-Guamanian motor neuron disease with neurofibrillary tangles, Pick's disease (PiD), postencephalitic parkinsonism, prion protein cerebral amyloid angiopathy, progressive subcortical gliosis, progressive supranuclear palsy (PSP), subacute sclerosing panencephalitis, tangle only dementia, Tangle-predominant dementia, multi-infarct dementia, ischemic stroke, or tuberous sclerosis.

25. A method of treating progressive supranuclear palsy (PSP) in a subject in need thereof, the method comprising administering to the subject the pharmaceutical composition according to claim 4.

26. A method of treating Alzheimer's disease in a subject in need thereof, the method comprising administering to the subject the pharmaceutical composition according to claim 4.

27. A method of treating frontotemporal dementia (FTD) in a subject in need thereof, the method comprising administering to the subject the pharmaceutical composition according to claim 4.

28. A method of treating corticobasal degeneration (CBD) in a subject in need thereof, the method comprising administering to the subject the pharmaceutical composition according to claim 4.

29. A cell containing the antisense oligonucleotide of claim 1.

30. A cell containing the antisense oligonucleotide of claim 2.


SEQUENCE LISTING (506 sequences)

SEQ ID NO: 1 - DNA, 18 bp, synthetic construct
  misc_feature 1..18: note = Description of Artificial Sequence: Syntheticoligonucleotide
  source 1..18: mol_type = other DNA
gtccactaacctttcagg

SEQ ID NO: 2 - DNA, 18 bp, synthetic construct
  misc_feature 1..18: note = Description of Artificial Sequence: Syntheticoligonucleotide
  source 1..18: mol_type = other DNA
gcatcgtcagcttacctt

SEQ ID NO: 3 - DNA, 18 bp, synthetic construct
  misc_feature 1..18: note = Description of Artificial Sequence: Syntheticoligonucleotide
  source 1..18: mol_type = other DNA
tatttgcacctggagatg

SEQ ID NO: 4 - DNA, 18 bp, synthetic construct
  misc_feature 1..18: note = Description of Artificial Sequence: Syntheticoligonucleotide
  source 1..18: mol_type = other DNA
gactatttgcacctggag

SEQ ID NO: 5 - DNA, 18 bp, synthetic construct
  misc_feature 1..18: note = Description of Artificial Sequence: Syntheticoligonucleotide
  source 1..18: mol_type = other DNA
catgcgagctgataaaat

SEQ ID NO: 6 - DNA, 18 bp, synthetic construct
  misc_feature 1..18: note = Description of Artificial Sequence: Syntheticoligonucleotide
  source 1..18: mol_type = other DNA
accatgcgagctgataaa

SEQ ID NO: 7 - DNA, 18 bp, synthetic construct
  misc_feature 1..18: note = Description of Artificial Sequence: Syntheticoligonucleotide
  source 1..18: mol_type = other DNA
ccatgcgagctgataaaa

SEQ ID NO: 8 - DNA, 18 bp, synthetic construct
  misc_feature 1..18: note = Description of Artificial Sequence: Syntheticoligonucleotide
  source 1..18: mol_type = other DNA
cgtcagcttaccttggct

SEQ ID NO: 9 - DNA, 18 bp, synthetic construct
  misc_feature 1..18: note = Description of Artificial Sequence: Syntheticoligonucleotide
  source 1..18: mol_type = other DNA
tgaccatgcgagctgata

SEQ ID NO: 10 - DNA, 18 bp, synthetic construct
  misc_feature 1..18: note = Description of Artificial Sequence: Syntheticoligonucleotide
  source 1..18: mol_type = other DNA
atgcgagctgataaaata

SEQ ID NO: 11 - DNA, 18 bp, synthetic construct
  misc_feature 1..18: note = Description of Artificial Sequence: Syntheticoligonucleotide
  source 1..18: mol_type = other DNA
tttgcacctggagatgag

SEQ ID NO: 12 - DNA, 18 bp, synthetic construct
  misc_feature 1..18: note = Description of Artificial Sequence: Syntheticoligonucleotide
  source 1..18: mol_type = other DNA
ttgcacctggagatgaga

SEQ ID NO: 13 - DNA, 18 bp, synthetic construct
  misc_feature 1..18: note = Description of Artificial Sequence: Syntheticoligonucleotide
  source 1..18: mol_type = other DNA
atttgcacctggagatga

SEQ ID NO: 14 - DNA, 18 bp, synthetic construct
  misc_feature 1..18: note = Description of Artificial Sequence: Syntheticoligonucleotide
  source 1..18: mol_type = other DNA
tccactaacctttcaggc

SEQ ID NO: 15 - DNA, 18 bp, synthetic construct
  misc_feature 1..18: note = Description of Artificial Sequence: Syntheticoligonucleotide
  source 1..18: mol_type = other DNA
ggtttcaatctgcaagaa

SEQ ID NO: 16 - DNA, 18 bp, synthetic construct
  misc_feature 1..18: note = Description of Artificial Sequence: Syntheticoligonucleotide
  source 1..18: mol_type = other DNA
ccactaacctttcaggcc

SEQ ID NO: 17 - DNA, 18 bp, synthetic construct
  misc_feature 1..18: note = Description of Artificial Sequence: Syntheticoligonucleotide
  source 1..18: mol_type = other DNA
cactaacctttcaggcca

SEQ ID NO: 18 - DNA, 18 bp, synthetic construct
  misc_feature 1..18: note = Description of Artificial Sequence: Syntheticoligonucleotide
  source 1..18: mol_type = other DNA
actaacctttcaggccag

SEQ ID NO: 19 - DNA, 18 bp, synthetic construct
  misc_feature 1..18: note = Description of Artificial Sequence: Syntheticoligonucleotide
  source 1..18: mol_type = other DNA
gctcagccatcctggttc

SEQ ID NO: 20 - DNA, 18 bp, synthetic construct
  misc_feature 1..18: note = Description of Artificial Sequence: Syntheticoligonucleotide
  source 1..18: mol_type = other DNA
gtttcaatctgcaagaag

SEQ ID NO: 21 - DNA, 18 bp, synthetic construct
  misc_feature 1..18: note = Description of Artificial Sequence: Syntheticoligonucleotide
  source 1..18: mol_type = other DNA
agttcacctggggaaaga

SEQ ID NO: 22 - DNA, 18 bp, synthetic construct
  misc_feature 1..18: note = Description of Artificial Sequence: Syntheticoligonucleotide
  source 1..18: mol_type = other DNA
ttggaggttcacctggga

SEQ ID NO: 23 - DNA, 18 bp, synthetic construct
  misc_feature 1..18: note = Description of Artificial Sequence: Syntheticoligonucleotide
  source 1..18: mol_type = other DNA
ggctacctggtttatgat

SEQ ID NO: 24 - DNA, 18 bp, synthetic construct
  misc_feature 1..18: note = Description of Artificial Sequence: Syntheticoligonucleotide
  source 1..18: mol_type = other DNA
aaagttcacctggggaaa

SEQ ID NO: 25 - DNA, 18 bp, synthetic construct
  misc_feature 1..18: note = Description of Artificial Sequence: Syntheticoligonucleotide
  source 1..18: mol_type = other DNA
gttcactgaccttgggtc

SEQ ID NO: 26 - DNA, 18 bp, synthetic construct
  misc_feature 1..18: note = Description of Artificial Sequence: Syntheticoligonucleotide
  source 1..18: mol_type = other DNA
caaagttcacctggggaa

SEQ ID NO: 27 - DNA, 18 bp, synthetic construct
  misc_feature 1..18: note = Description of Artificial Sequence: Syntheticoligonucleotide
  source 1..18: mol_type = other DNA
cagcttaccttggctttt

SEQ ID NO: 28 - DNA, 18 bp, synthetic construct
  misc_feature 1..18: note = Description of Artificial Sequence: Syntheticoligonucleotide
  source 1..18: mol_type = other DNA
gggctacctggtttatga

SEQ ID NO: 29 - DNA, 18 bp, synthetic construct
  misc_feature 1..18: note = Description of Artificial Sequence: Syntheticoligonucleotide
  source 1..18: mol_type = other DNA
tcttcagctggtgtatgt

SEQ ID NO: 30 - DNA, 18 bp, synthetic construct
  misc_feature 1..18: note = Description of Artificial Sequence: Syntheticoligonucleotide
  source 1..18: mol_type = other DNA
ttcaaagttcacctgggg

SEQ ID NO: 31 - DNA, 18 bp, synthetic construct
  misc_feature 1..18: note = Description of Artificial Sequence: Syntheticoligonucleotide
  source 1..18: mol_type = other DNA
ccctttacctttttattt

SEQ ID NO: 32 - DNA, 18 bp, synthetic construct
  misc_feature 1..18: note = Description of Artificial Sequence: Syntheticoligonucleotide
  source 1..18: mol_type = other DNA
tgcttcttcagctggtgt

SEQ ID NO: 33 - DNA, 18 bp, synthetic construct
  misc_feature 1..18: note = Description of Artificial Sequence: Syntheticoligonucleotide
  source 1..18: mol_type = other DNA
tcagcttaccttggcttt

SEQ ID NO: 34 - DNA, 18 bp, synthetic construct
  misc_feature 1..18: note = Description of Artificial Sequence: Syntheticoligonucleotide
  source 1..18: mol_type = other DNA
ctgcttcttcagctggtg

SEQ ID NO: 35 - DNA, 18 bp, synthetic construct
  misc_feature 1..18: note = Description of Artificial Sequence: Syntheticoligonucleotide
  source 1..18: mol_type = other DNA
ggccacctcctagaacac

SEQ ID NO: 36 - DNA, 18 bp, synthetic construct
  misc_feature 1..18: note = Description of Artificial Sequence: Syntheticoligonucleotide
  source 1..18: mol_type = other DNA
tcttaccagagctgggtg

SEQ ID NO: 37 - DNA, 18 bp, synthetic construct
  misc_feature 1..18: note = Description of Artificial Sequence: Syntheticoligonucleotide
  source 1..18: mol_type = other DNA
aagttcacctggggaaag

SEQ ID NO: 38 - DNA, 18 bp, synthetic construct
  misc_feature 1..18: note = Description of Artificial Sequence: Syntheticoligonucleotide
  source 1..18: mol_type = other DNA
gtcagcttaccttggctt

SEQ ID NO: 39 - DNA, 18 bp, synthetic construct
  misc_feature 1..18: note = Description of Artificial Sequence: Syntheticoligonucleotide
  source 1..18: mol_type = other DNA
ggggcctgatcacaaacc

SEQ ID NO: 40 - DNA, 18 bp, synthetic construct
  misc_feature 1..18: note = Description of Artificial Sequence: Syntheticoligonucleotide
  source 1..18: mol_type = other DNA
aggttcacctgggaagga

SEQ ID NO: 41 - DNA, 18 bp, synthetic construct
  misc_feature 1..18: note = Description of Artificial Sequence: Syntheticoligonucleotide
  source 1..18: mol_type = other DNA
gcttaccttggctttttt

SEQ ID NO: 42 - DNA, 18 bp, synthetic construct
  misc_feature 1..18: note = Description of Artificial Sequence: Syntheticoligonucleotide
  source 1..18: mol_type = other DNA
tcaaagttcacctgggga

SEQ ID NO: 43 - DNA, 18 bp, synthetic construct
  misc_feature 1..18: note = Description of Artificial Sequence: Syntheticoligonucleotide
  source 1..18: mol_type = other DNA
ccactctcaccttcccgc

SEQ ID NO: 44 - DNA, 18 bp, synthetic construct
  misc_feature 1..18: note = Description of Artificial Sequence: Syntheticoligonucleotide
  source 1..18: mol_type = other DNA
ccccctttacctttttat

SEQ ID NO: 45 - DNA, 18 bp, synthetic construct
  misc_feature 1..18: note = Description of Artificial Sequence: Syntheticoligonucleotide
  source 1..18: mol_type = other DNA
gaggttcacctgggaagg

SEQ ID NO: 46 - DNA, 18 bp, synthetic construct
  misc_feature 1..18: note = Description of Artificial Sequence: Syntheticoligonucleotide
  source 1..18: mol_type = other DNA
gttcacctgggaaggaag

SEQ ID NO: 47 - DNA, 18 bp, synthetic construct
  misc_feature 1..18: note = Description of Artificial Sequence: Syntheticoligonucleotide
  source 1..18: mol_type = other DNA
cacctcctagaacacaac

SEQ ID NO: 48 - DNA, 18 bp, synthetic construct
  misc_feature 1..18: note = Description of Artificial Sequence: Syntheticoligonucleotide
  source 1..18: mol_type = other DNA
actctcaccttcccgcct

SEQ ID NO: 49 - DNA, 18 bp, synthetic construct
  misc_feature 1..18: note = Description of Artificial Sequence: Syntheticoligonucleotide
  source 1..18: mol_type = other DNA
ttcaatctgcaagaagag

SEQ ID NO: 50 - DNA, 18 bp, synthetic construct
  misc_feature 1..18: note = Description of Artificial Sequence: Syntheticoligonucleotide
  source 1..18: mol_type = other DNA
actgaccttgggtcacgt

SEQ ID NO: 51 - DNA, 18 bp, synthetic construct
  misc_feature 1..18: note = Description of Artificial Sequence: Syntheticoligonucleotide
  source 1..18: mol_type = other DNA
tttcaatctgcaagaaga

SEQ ID NO: 52 - DNA, 18 bp, synthetic construct
  misc_feature 1..18: note = Description of Artificial Sequence: Syntheticoligonucleotide
  source 1..18: mol_type = other DNA
ttcttaccagagctgggt

SEQ ID NO: 53 - DNA, 18 bp, synthetic construct
  misc_feature 1..18: note = Description of Artificial Sequence: Syntheticoligonucleotide
  source 1..18: mol_type = other DNA
cagggctacctggtttat

SEQ ID NO: 54 - DNA, 18 bp, synthetic construct
  misc_feature 1..18: note = Description of Artificial Sequence: Syntheticoligonucleotide
  source 1..18: mol_type = other DNA
gggcctgatcacaaaccc

SEQ ID NO: 55 - DNA, 18 bp, synthetic construct
  misc_feature 1..18: note = Description of Artificial Sequence: Syntheticoligonucleotide
  source 1..18: mol_type = other DNA
agggctacctggtttatg

SEQ ID NO: 56 - DNA, 18 bp, synthetic construct
  misc_feature 1..18: note = Description of Artificial Sequence: Syntheticoligonucleotide
  source 1..18: mol_type = other DNA
ccacctcctagaacacaa

SEQ ID NO: 57 - DNA, 18 bp, synthetic construct
  misc_feature 1..18: note = Description of Artificial Sequence: Syntheticoligonucleotide
  source 1..18: mol_type = other DNA
cactgaccttgggtcacg

SEQ ID NO: 58 - DNA, 18 bp, synthetic construct
  misc_feature 1..18: note = Description of Artificial Sequence: Syntheticoligonucleotide
  source 1..18: mol_type = other DNA
cccctttacctttttatt

SEQ ID NO: 59 - DNA, 18 bp, synthetic construct
  misc_feature 1..18: note = Description of Artificial Sequence: Syntheticoligonucleotide
  source 1..18: mol_type = other DNA
ttcactgaccttgggtca

SEQ ID NO: 60 - DNA, 18 bp, synthetic construct
  misc_feature 1..18: note = Description of Artificial Sequence: Syntheticoligonucleotide
  source 1..18: mol_type = other DNA
ggcctgatcacaaaccct

SEQ ID NO: 61 - DNA, 18 bp, synthetic construct
  misc_feature 1..18: note = Description of Artificial Sequence: Syntheticoligonucleotide
  source 1..18: mol_type = other DNA
cactctcaccttcccgcc

SEQ ID NO: 62 - DNA, 18 bp, synthetic construct
  misc_feature 1..18: note = Description of Artificial Sequence: Syntheticoligonucleotide
  source 1..18: mol_type = other DNA
cctggccacctcctagaa

SEQ ID NO: 63 - DNA, 18 bp, synthetic construct
  misc_feature 1..18: note = Description of Artificial Sequence: Syntheticoligonucleotide
  source 1..18: mol_type = other DNA
cctttacctttttatttc

SEQ ID NO: 64 - DNA, 18 bp, synthetic construct
  misc_feature 1..18: note = Description of Artificial Sequence: Syntheticoligonucleotide
  source 1..18: mol_type = other DNA
tcactgaccttgggtcac

SEQ ID NO: 65 - DNA, 18 bp, synthetic construct
  misc_feature 1..18: note = Description of Artificial Sequence: Syntheticoligonucleotide
  source 1..18: mol_type = other DNA
gcctgatcacaaaccctg

SEQ ID NO: 66 - DNA, 18 bp, synthetic construct
  misc_feature 1..18: note = Description of Artificial Sequence: Syntheticoligonucleotide
  source 1..18: mol_type = other DNA
ctttacctttttatttcc

SEQ ID NO: 67 - DNA, 18 bp, synthetic construct
  misc_feature 1..18: note = Description of Artificial Sequence: Syntheticoligonucleotide
  source 1..18: mol_type = other DNA
ttcttcagctggtgtatg

SEQ ID NO: 68 - DNA, 18 bp, synthetic construct
  misc_feature 1..18: note = Description of Artificial Sequence: Syntheticoligonucleotide
  source 1..18: mol_type = other DNA
gccacctcctagaacaca

SEQ ID NO: 69 - DNA, 18 bp, synthetic construct
  misc_feature 1..18: note = Description of Artificial Sequence: Syntheticoligonucleotide
  source 1..18: mol_type = other DNA
tctcaccttcccgcctcc

SEQ ID NO: 70 - DNA, 18 bp, synthetic construct
  misc_feature 1..18: note = Description of Artificial Sequence: Syntheticoligonucleotide
  source 1..18: mol_type = other DNA
cttcttaccagagctggg

SEQ ID NO: 71 - DNA, 18 bp, synthetic construct
  misc_feature 1..18: note = Description of Artificial Sequence: Syntheticoligonucleotide
  source 1..18: mol_type = other DNA
ttcttcttaccagagctg

SEQ ID NO: 72 - DNA, 18 bp, synthetic construct
  misc_feature 1..18: note = Description of Artificial Sequence: Syntheticoligonucleotide
  source 1..18: mol_type = other DNA
atcagccccctgtaaatg

SEQ ID NO: 73 - DNA, 18 bp, synthetic construct
  misc_feature 1..18: note = Description of Artificial Sequence: Syntheticoligonucleotide
  source 1..18: mol_type = other DNA
gcttcttcagctggtgta

SEQ ID NO: 74 - DNA, 18 bp, synthetic construct
  misc_feature 1..18: note = Description of Artificial Sequence: Syntheticoligonucleotide
  source 1..18: mol_type = other DNA
acagggctacctggttta

SEQ ID NO: 75 - DNA, 18 bp, synthetic construct
  misc_feature 1..18: note = Description of Artificial Sequence: Syntheticoligonucleotide
  source 1..18: mol_type = other DNA
ctcagccatcctggttca

SEQ ID NO: 76 - DNA, 18 bp, synthetic construct
  misc_feature 1..18: note = Description of Artificial Sequence: Syntheticoligonucleotide
  source 1..18: mol_type = other DNA
cagccccctgtaaatgaa

SEQ ID NO: 77 - DNA, 18 bp, synthetic construct
  misc_feature 1..18: note = Description of Artificial Sequence: Syntheticoligonucleotide
  source 1..18: mol_type = other DNA
gggctcagccatcctggt

SEQ ID NO: 78 - DNA, 18 bp, synthetic construct
  misc_feature 1..18: note = Description of Artificial Sequence: Syntheticoligonucleotide
  source 1..18: mol_type = other DNA
tcttcttaccagagctgg

SEQ ID NO: 79 - DNA, 18 bp, synthetic construct
  misc_feature 1..18: note = Description of Artificial Sequence: Syntheticoligonucleotide
  source 1..18: mol_type = other DNA
ctctcaccttcccgcctc

SEQ ID NO: 80 - DNA, 18 bp, synthetic construct
  misc_feature 1..18: note = Description of Artificial Sequence: Syntheticoligonucleotide
  source 1..18: mol_type = other DNA
tcagccccctgtaaatga

SEQ ID NO: 81 - DNA, 18 bp, synthetic construct
  misc_feature 1..18: note = Description of Artificial Sequence: Syntheticoligonucleotide
  source 1..18: mol_type = other DNA
cttcttcagctggtgtat

SEQ ID NO: 82 - DNA, 18 bp, synthetic construct
  misc_feature 1..18: note = Description of Artificial Sequence: Syntheticoligonucleotide
  source 1..18: mol_type = other DNA
ggttcacctgggaaggaa

SEQ ID NO: 83 - DNA, 18 bp, synthetic construct
  misc_feature 1..18: note = Description of Artificial Sequence: Syntheticoligonucleotide
  source 1..18: mol_type = other DNA
catcagccccctgtaaat

SEQ ID NO: 84 - DNA, 18 bp, synthetic construct
  misc_feature 1..18: note = Description of Artificial Sequence: Syntheticoligonucleotide
  source 1..18: mol_type = other DNA
accatcagccccctgtaa

SEQ ID NO: 85 - DNA, 25 bp, synthetic construct
  misc_feature 1..25: note = Description of Artificial Sequence: Syntheticoligonucleotide
  source 1..25: mol_type = other DNA
gtccactaacctttcaggccgtgtc

SEQ ID NO: 86 - DNA, 24 bp, synthetic construct
  misc_feature 1..24: note = Description of Artificial Sequence: Syntheticoligonucleotide
  source 1..24: mol_type = other DNA
gtccactaacctttcaggccgtgt

SEQ ID NO: 87 - DNA, 23 bp, synthetic construct
  misc_feature 1..23: note = Description of Artificial Sequence: Syntheticoligonucleotide
  source 1..23: mol_type = other DNA
gtccactaacctttcaggccgtg

SEQ ID NO: 88 - DNA, 22 bp, synthetic construct
  misc_feature 1..22: note = Description of Artificial Sequence: Syntheticoligonucleotide
  source 1..22: mol_type = other DNA
gtccactaacctttcaggccgt

SEQ ID NO: 89 - DNA, 21 bp, synthetic construct
  misc_feature 1..21: note = Description of Artificial Sequence: Syntheticoligonucleotide
  source 1..21: mol_type = other DNA
gtccactaacctttcaggccg

SEQ ID NO: 90 - DNA, 20 bp, synthetic construct
  misc_feature 1..20: note = Description of Artificial Sequence: Syntheticoligonucleotide
  source 1..20: mol_type = other DNA
gtccactaacctttcaggcc

SEQ ID NO: 91 - DNA, 19 bp, synthetic construct
  misc_feature 1..19: note = Description of Artificial Sequence: Syntheticoligonucleotide
  source 1..19: mol_type = other DNA
gtccactaacctttcaggc

SEQ ID NO: 92 - DNA, 18 bp, synthetic construct
  misc_feature 1..18: note = Description of Artificial Sequence: Syntheticoligonucleotide
  source 1..18: mol_type = other DNA
gtccactaacctttcagg

SEQ ID NO: 93 - DNA, 17 bp, synthetic construct
  misc_feature 1..17: note = Description of Artificial Sequence: Syntheticoligonucleotide
  source 1..17: mol_type = other DNA
gtccactaacctttcag

SEQ ID NO: 94 - DNA, 16 bp, synthetic construct
  misc_feature 1..16: note = Description of Artificial Sequence: Syntheticoligonucleotide
  source 1..16: mol_type = other DNA
gtccactaacctttca

SEQ ID NO: 95 - DNA, 15 bp, synthetic construct
  misc_feature 1..15: note = Description of Artificial Sequence: Syntheticoligonucleotide
  source 1..15: mol_type = other DNA
gtccactaacctttc

SEQ ID NO: 96 - DNA, 14 bp, synthetic construct
  misc_feature 1..14: note = Description of Artificial Sequence: Syntheticoligonucleotide
  source 1..14: mol_type = other DNA
gtccactaaccttt

SEQ ID NO: 97 - DNA, 13 bp, synthetic construct
  misc_feature 1..13: note = Description of Artificial Sequence: Syntheticoligonucleotide
  source 1..13: mol_type = other DNA
gtccactaacctt

SEQ ID NO: 98 - DNA, 12 bp, synthetic construct
  misc_feature 1..12: note = Description of Artificial Sequence: Syntheticoligonucleotide
  source 1..12: mol_type = other DNA
gtccactaacct

SEQ ID NO: 99 - DNA, 25 bp, synthetic construct
  misc_feature 1..25: note = Description of Artificial Sequence: Syntheticoligonucleotide
  source 1..25: mol_type = other DNA
gcatcgtcagcttaccttggctttt

SEQ ID NO: 100 - DNA, 24 bp, synthetic construct
  misc_feature 1..24: note = Description of Artificial Sequence: Syntheticoligonucleotide
  source 1..24: mol_type = other DNA
gcatcgtcagcttaccttggcttt

SEQ ID NO: 101 - DNA, 23 bp, synthetic construct
  misc_feature 1..23: note = Description of Artificial Sequence: Syntheticoligonucleotide
  source 1..23: mol_type = other DNA
gcatcgtcagcttaccttggctt

SEQ ID NO: 102 - DNA, 22 bp, synthetic construct
  misc_feature 1..22: note = Description of Artificial Sequence: Syntheticoligonucleotide
  source 1..22: mol_type = other DNA
gcatcgtcagcttaccttggct

SEQ ID NO: 103 - DNA, 21 bp, synthetic construct
  misc_feature 1..21: note = Description of Artificial Sequence: Syntheticoligonucleotide
  source 1..21: mol_type = other DNA
gcatcgtcagcttaccttggc

SEQ ID NO: 104 - DNA, 20 bp, synthetic construct
  misc_feature 1..20: note = Description of Artificial Sequence: Syntheticoligonucleotide
  source 1..20: mol_type = other DNA
gcatcgtcagcttaccttgg

SEQ ID NO: 105 - DNA, 19 bp, synthetic construct
  misc_feature 1..19: note = Description of Artificial Sequence: Syntheticoligonucleotide
  source 1..19: mol_type = other DNA
gcatcgtcagcttaccttg

SEQ ID NO: 106 - DNA, 18 bp, synthetic construct
  misc_feature 1..18: note = Description of Artificial Sequence: Syntheticoligonucleotide
  source 1..18: mol_type = other DNA
gcatcgtcagcttacctt

SEQ ID NO: 107 - DNA, 17 bp, synthetic construct
  misc_feature 1..17: note = Description of Artificial Sequence: Syntheticoligonucleotide
  source 1..17: mol_type = other DNA
gcatcgtcagcttacct

SEQ ID NO: 108 - DNA, 16 bp, synthetic construct
  misc_feature 1..16: note = Description of Artificial Sequence: Syntheticoligonucleotide
  source 1..16: mol_type = other DNA
gcatcgtcagcttacc

SEQ ID NO: 109 - DNA, 15 bp, synthetic construct
  misc_feature 1..15: note = Description of Artificial Sequence: Syntheticoligonucleotide
  source 1..15: mol_type = other DNA
gcatcgtcagcttac

SEQ ID NO: 110 - DNA, 14 bp, synthetic construct
  misc_feature 1..14: note = Description of Artificial Sequence: Syntheticoligonucleotide
  source 1..14: mol_type = other DNA
gcatcgtcagctta

SEQ ID NO: 111 - DNA, 13 bp, synthetic construct
  misc_feature 1..13: note = Description of Artificial Sequence: Syntheticoligonucleotide
  source 1..13: mol_type = other DNA
gcatcgtcagctt

SEQ ID NO: 112 - DNA, 12 bp, synthetic construct
  misc_feature 1..12: note = Description of Artificial Sequence: Syntheticoligonucleotide
  source 1..12: mol_type = other DNA
gcatcgtcagct

SEQ ID NO: 113 - DNA, 25 bp, synthetic construct
  misc_feature 1..25: note = Description of Artificial Sequence: Syntheticoligonucleotide
  source 1..25: mol_type = other DNA
gactatttgcacctggagatgagag

SEQ ID NO: 114 - DNA, 24 bp, synthetic construct
  misc_feature 1..24: note = Description of Artificial Sequence: Syntheticoligonucleotide
  source 1..24: mol_type = other DNA
gactatttgcacctggagatgaga

SEQ ID NO: 115 - DNA, 23 bp, synthetic construct
  misc_feature 1..23: note = Description of Artificial Sequence: Syntheticoligonucleotide
  source 1..23: mol_type = other DNA
gactatttgcacctggagatgag

SEQ ID NO: 116 - DNA, 22 bp, synthetic construct
  misc_feature 1..22: note = Description of Artificial Sequence: Syntheticoligonucleotide
  source 1..22: mol_type = other DNA
gactatttgcacctggagatga

SEQ ID NO: 117 - DNA, 21 bp, synthetic construct
  misc_feature 1..21: note = Description of Artificial Sequence: Syntheticoligonucleotide
  source 1..21: mol_type = other DNA
gactatttgcacctggagatg

SEQ ID NO: 118 - DNA, 20 bp, synthetic construct
  misc_feature 1..20: note = Description of Artificial Sequence: Syntheticoligonucleotide
  source 1..20: mol_type = other DNA
gactatttgcacctggagat

SEQ ID NO: 119 - DNA, 19 bp, synthetic construct
  misc_feature 1..19: note = Description of Artificial Sequence: Syntheticoligonucleotide
  source 1..19: mol_type = other DNA
gactatttgcacctggaga

SEQ ID NO: 120 - DNA, 18 bp, synthetic construct
  misc_feature 1..18: note = Description of Artificial Sequence: Syntheticoligonucleotide
  source 1..18: mol_type = other DNA
gactatttgcacctggag

SEQ ID NO: 121 - DNA, 17 bp, synthetic construct
  misc_feature 1..17: note = Description of Artificial Sequence: Syntheticoligonucleotide
  source 1..17: mol_type = other DNA
gactatttgcacctgga

SEQ ID NO: 122 - DNA, 16 bp, synthetic construct
  misc_feature 1..16: note = Description of Artificial Sequence: Syntheticoligonucleotide
  source 1..16: mol_type = other DNA
gactatttgcacctgg

SEQ ID NO: 123 - DNA, 15 bp, synthetic construct
  misc_feature 1..15: note = Description of Artificial Sequence: Syntheticoligonucleotide
  source 1..15: mol_type = other DNA
gactatttgcacctg

SEQ ID NO: 124 - DNA, 14 bp, synthetic construct
  misc_feature 1..14: note = Description of Artificial Sequence: Syntheticoligonucleotide
  source 1..14: mol_type = other DNA
gactatttgcacct

SEQ ID NO: 125 - DNA, 13 bp, synthetic construct
  misc_feature 1..13: note = Description of Artificial Sequence: Syntheticoligonucleotide
  source 1..13: mol_type = other DNA
gactatttgcacc

SEQ ID NO: 126 - DNA, 12 bp, synthetic construct
  misc_feature 1..12: note = Description of Artificial Sequence: Syntheticoligonucleotide
  source 1..12: mol_type = other DNA
gactatttgcac

SEQ ID NO: 127 - DNA, 25 bp, synthetic construct
  misc_feature 1..25: note = Description of Artificial Sequence: Syntheticoligonucleotide
  source 1..25: mol_type = other DNA
ccatgcgagctgataaaatataaaa

SEQ ID NO: 128 - DNA, 24 bp, synthetic construct
  misc_feature 1..24: note = Description of Artificial Sequence: Syntheticoligonucleotide
  source 1..24: mol_type = other DNA
ccatgcgagctgataaaatataaa

SEQ ID NO: 129 - DNA, 23 bp, synthetic construct
  misc_feature 1..23: note = Description of Artificial Sequence: Syntheticoligonucleotide
  source 1..23: mol_type = other DNA
ccatgcgagctgataaaatataa

SEQ ID NO: 130 - DNA, 22 bp, synthetic construct
  misc_feature 1..22: note = Description of Artificial Sequence: Syntheticoligonucleotide
  source 1..22: mol_type = other DNA
ccatgcgagctgataaaatata

SEQ ID NO: 131 - DNA, 21 bp, synthetic construct
  misc_feature 1..21: note = Description of Artificial Sequence: Syntheticoligonucleotide
  source 1..21: mol_type = other DNA
ccatgcgagctgataaaatat

SEQ ID NO: 132 - DNA, 20 bp, synthetic construct
  misc_feature 1..20: note = Description of Artificial Sequence: Syntheticoligonucleotide
  source 1..20: mol_type = other DNA
ccatgcgagctgataaaata

SEQ ID NO: 133 - DNA, 19 bp, synthetic construct
  misc_feature 1..19: note = Description of Artificial Sequence: Syntheticoligonucleotide
  source 1..19: mol_type = other DNA
ccatgcgagctgataaaat

SEQ ID NO: 134 - DNA, 18 bp, synthetic construct
  misc_feature 1..18: note = Description of Artificial Sequence: Syntheticoligonucleotide
  source 1..18: mol_type = other DNA
ccatgcgagctgataaaa

SEQ ID NO: 135 - DNA, 17 bp, synthetic construct
  misc_feature 1..17: note = Description of Artificial Sequence: Syntheticoligonucleotide
  source 1..17: mol_type = other DNA
ccatgcgagctgataaa

SEQ ID NO: 136 - DNA, 16 bp, synthetic construct
  misc_feature 1..16: note = Description of Artificial Sequence: Syntheticoligonucleotide
  source 1..16: mol_type = other DNA
ccatgcgagctgataa

SEQ ID NO: 137 - DNA, 15 bp, synthetic construct
  misc_feature 1..15: note = Description of Artificial Sequence: Syntheticoligonucleotide
  source 1..15: mol_type = other DNA
ccatgcgagctgata

SEQ ID NO: 138 - DNA, 14 bp, synthetic construct
  misc_feature 1..14: note = Description of Artificial Sequence: Syntheticoligonucleotide
  source 1..14: mol_type = other DNA
ccatgcgagctgat

SEQ ID NO: 139 - DNA, 13 bp, synthetic construct
  misc_feature 1..13: note = Description of Artificial Sequence: Syntheticoligonucleotide
  source 1..13: mol_type = other DNA
ccatgcgagctga

SEQ ID NO: 140 - DNA, 12 bp, synthetic construct
  misc_feature 1..12: note = Description of Artificial Sequence: Syntheticoligonucleotide
  source 1..12: mol_type = other DNA
ccatgcgagctg

SEQ ID NO: 141 - DNA, 17 bp, synthetic construct
  misc_feature 1..17: note = Description of Artificial Sequence: Syntheticoligonucleotide
  source 1..17: mol_type = other DNA
agccatcctggttcaaa

SEQ ID NO: 142 - DNA, 17 bp, synthetic construct
  misc_feature 1..17: note = Description of Artificial Sequence: Syntheticoligonucleotide
  source 1..17: mol_type = other DNA
cagccatcctggttcaa

SEQ ID NO: 143 - DNA, 17 bp, synthetic construct
  misc_feature 1..17: note = Description of Artificial Sequence: Syntheticoligonucleotide
  source 1..17: mol_type = other DNA
tcagccatcctggttca

SEQ ID NO: 144 - DNA, 17 bp, synthetic construct
  misc_feature 1..17: note = Description of Artificial Sequence: Syntheticoligonucleotide
  source 1..17: mol_type = other DNA
ctcagccatcctggttc

SEQ ID NO: 145 - DNA, 17 bp, synthetic construct
  misc_feature 1..17: note = Description of Artificial Sequence: Syntheticoligonucleotide
  source 1..17: mol_type = other DNA
ggccagcgtccgtgtca

SEQ ID NO: 146 - DNA, 17 bp, synthetic construct
  misc_feature 1..17: note = Description of Artificial Sequence: Syntheticoligonucleotide
  source 1..17: mol_type = other DNA
gccagcgtccgtgtcac

SEQ ID NO: 147 - DNA, 17 bp, synthetic construct
  misc_feature 1..17: note = Description of Artificial Sequence: Syntheticoligonucleotide
  source 1..17: mol_type = other DNA
ccagcgtccgtgtcacc

SEQ ID NO: 148 - DNA, 17 bp, synthetic construct
  misc_feature 1..17: note = Description of Artificial Sequence: Syntheticoligonucleotide
  source 1..17: mol_type = other DNA
cagcgtccgtgtcaccc

SEQ ID NO: 149 - DNA, 17 bp, synthetic construct
  misc_feature 1..17: note = Description of Artificial Sequence: Syntheticoligonucleotide
  source 1..17: mol_type = other DNA
gtctccaatgcctgctt

SEQ ID NO: 150 - DNA, 17 bp, synthetic construct
  misc_feature 1..17: note = Description of Artificial Sequence: Syntheticoligonucleotide
  source 1..17: mol_type = other DNA
tgtctccaatgcctgct

SEQ ID NO: 151 - DNA, 17 bp, synthetic construct
  misc_feature 1..17: note = Description of Artificial Sequence: Syntheticoligonucleotide
  source 1..17: mol_type = other DNA
gtgtctccaatgcctgc

SEQ ID NO: 152 - DNA, 17 bp, synthetic construct
  misc_feature 1..17: note = Description of Artificial Sequence: Syntheticoligonucleotide
  source 1..17: mol_type = other DNA
ggtgtctccaatgcctg

SEQ ID NO: 153 - DNA, 17 bp, synthetic construct
  misc_feature 1..17: note = Description of Artificial Sequence: Syntheticoligonucleotide
  source 1..17: mol_type = other DNA
tcacgtgaccagcagct

SEQ ID NO: 154 - DNA, 17 bp, synthetic construct
  misc_feature 1..17: note = Description of Artificial Sequence: Syntheticoligonucleotide
  source 1..17: mol_type = other DNA
cacgtgaccagcagctt

SEQ ID NO: 155 - DNA, 17 bp, synthetic construct
  misc_feature 1..17: note = Description of Artificial Sequence: Syntheticoligonucleotide
  source 1..17: mol_type = other DNA
acgtgaccagcagcttc

SEQ ID NO: 156 - DNA, 17 bp, synthetic construct
  misc_feature 1..17: note = Description of Artificial Sequence: Syntheticoligonucleotide
  source 1..17: mol_type = other DNA
cgaagctgctggtcacg

SEQ ID NO: 157 - DNA, 17 bp, synthetic construct
  misc_feature 1..17: note = Description of Artificial Sequence: Syntheticoligonucleotide
  source 1..17: mol_type = other DNA
tttgcttttactgacca

SEQ ID NO: 158 - DNA, 17 bp, synthetic construct
  misc_feature 1..17: note = Description of Artificial Sequence: Syntheticoligonucleotide
  source 1..17: mol_type = other DNA
ctttgcttttactgacc

SEQ ID NO: 159 - DNA, 17 bp, synthetic construct
  misc_feature 1..17: note = Description of Artificial Sequence: Syntheticoligonucleotide
  source 1..17: mol_type = other DNA
tctttgcttttactgac

SEQ ID NO: 160 - DNA, 17 bp, synthetic construct
  misc_feature 1..17: note = Description of Artificial Sequence: Syntheticoligonucleotide
  source 1..17: mol_type = other DNA
gtctttgcttttactga

SEQ ID NO: 161 - DNA, 17 bp, synthetic construct
  misc_feature 1..17: note = Description of Artificial Sequence: Syntheticoligonucleotide
  source 1..17: mol_type = other DNA
ttttttgtcatcgcttc

SEQ ID NO: 162 - DNA, 17 bp, synthetic construct
  misc_feature 1..17: note = Description of Artificial Sequence: Syntheticoligonucleotide
  source 1..17: mol_type = other DNA
tttttgtcatcgcttcc

SEQ ID NO: 163 - DNA, 17 bp, synthetic construct
  misc_feature 1..17: note = Description of Artificial Sequence: Syntheticoligonucleotide
  source 1..17: mol_type = other DNA
ttttgtcatcgcttcca

SEQ ID NO: 164 - DNA, 17 bp, synthetic construct
  misc_feature 1..17: note = Description of Artificial Sequence: Syntheticoligonucleotide
  source 1..17: mol_type = other DNA
tttgtcatcgcttccag

SEQ ID NO: 165 - DNA, 17 bp, synthetic construct
  misc_feature 1..17: note = Description of Artificial Sequence: Syntheticoligonucleotide
  source 1..17: mol_type = other DNA
atcttcgttttaccatc

SEQ ID NO: 166 - DNA, 17 bp, synthetic construct
  misc_feature 1..17: note = Description of Artificial Sequence: Syntheticoligonucleotide
  source 1..17: mol_type = other DNA
gatcttcgttttaccat

SEQ ID NO: 167 - DNA, 17 bp, synthetic construct
  misc_feature 1..17: note = Description of Artificial Sequence: Syntheticoligonucleotide
  source 1..17: mol_type = other DNA
cgatcttcgttttacca

SEQ ID NO: 168 - DNA, 17 bp, synthetic construct
  misc_feature 1..17: note = Description of Artificial Sequence: Syntheticoligonucleotide
  source 1..17: mol_type = other DNA
gcgatcttcgttttacc

SEQ ID NO: 169 - DNA, 17 bp, synthetic construct
  misc_feature 1..17: note = Description of Artificial Sequence: Syntheticoligonucleotide
  source 1..17: mol_type = other DNA
tgggtggtgtctttgga

SEQ ID NO: 170 - DNA, 17 bp, synthetic construct
  misc_feature 1..17: note = Description of Artificial Sequence: Syntheticoligonucleotide
  source 1..17: mol_type = other DNA
gggtggtgtctttggag

SEQ ID NO: 171 - DNA, 17 bp, synthetic construct
  misc_feature 1..17: note = Description of Artificial Sequence: Syntheticoligonucleotide
  source 1..17: mol_type = other DNA
ggtggtgtctttggagc

SEQ ID NO: 172 - DNA, 17 bp, synthetic construct
  misc_feature 1..17: note = Description of Artificial Sequence: Syntheticoligonucleotide
  source 1..17: mol_type = other DNA
gtggtgtctttggagcg

SEQ ID NO: 173 - DNA, 17 bp, synthetic construct
  misc_feature 1..17: note = Description of Artificial Sequence: Syntheticoligonucleotide
  source 1..17: mol_type = other DNA
atcccctgattttggag

SEQ ID NO: 174 - DNA, 17 bp, synthetic construct
  misc_feature 1..17: note = Description of Artificial Sequence: Syntheticoligonucleotide
  source 1..17: mol_type = other DNA
gatcccctgattttgga

SEQ ID NO: 175 - DNA, 17 bp, synthetic construct
  misc_feature 1..17: note = Description of Artificial Sequence: Syntheticoligonucleotide
  source 1..17: mol_type = other DNA
cgatcccctgattttgg

SEQ ID NO: 176 - DNA, 17 bp, synthetic construct
  misc_feature 1..17: note = Description of Artificial Sequence: Syntheticoligonucleotide
  source 1..17: mol_type = other DNA
gcgatcccctgattttg

SEQ ID NO: 177 - DNA, 17 bp, synthetic construct
  misc_feature 1..17: note = Description of Artificial Sequence: Syntheticoligonucleotide
  source 1..17: mol_type = other DNA
gcctcccggctggtgct

SEQ ID NO: 178 - DNA, 17 bp, synthetic construct
  misc_feature 1..17: note = Description of Artificial Sequence: Syntheticoligonucleotide
  source 1..17: mol_type = other DNA
cctcccggctggtgctt

SEQ ID NO: 179 - DNA, 17 bp, synthetic construct
  misc_feature 1..17: note = Description of Artificial Sequence: Syntheticoligonucleotide
  source 1..17: mol_type = other DNA
ctcccggctggtgcttc

SEQ ID NO: 180 - DNA, 17 bp, synthetic construct
  misc_feature 1..17: note = Description of Artificial Sequence: Syntheticoligonucleotide
  source 1..17: mol_type = other DNA
tcccggctggtgcttca

SEQ ID NO: 181 - DNA, 17 bp, synthetic construct
  misc_feature 1..17: note = Description of Artificial Sequence: Syntheticoligonucleotide
  source 1..17: mol_type = other DNA
actggtttgtagactat

SEQ ID NO: 182 - DNA, 17 bp, synthetic construct
  misc_feature 1..17: note = Description of Artificial Sequence: Syntheticoligonucleotide
  source 1..17: mol_type = other DNA
aactggtttgtagacta

SEQ ID NO: 183 - DNA, 17 bp, synthetic construct
  misc_feature 1..17: note = Description of Artificial Sequence: Syntheticoligonucleotide
  source 1..17: mol_type = other DNA
caactggtttgtagact

SEQ ID NO: 184 - DNA, 17 bp, synthetic construct
  misc_feature 1..17: note = Description of Artificial Sequence: Syntheticoligonucleotide
  source 1..17: mol_type = other DNA
tcaactggtttgtagac

SEQ ID NO: 185 - DNA, 17 bp, synthetic construct
  misc_feature 1..17: note = Description of Artificial Sequence: Syntheticoligonucleotide
  source 1..17: mol_type = other DNA
tatgatggatgttgcct

SEQ ID NO: 186 - DNA, 17 bp, synthetic construct
  misc_feature 1..17: note = Description of Artificial Sequence: Syntheticoligonucleotide
  source 1..17: mol_type = other DNA
atgatggatgttgccta

SEQ ID NO: 187 - DNA, 17 bp, synthetic construct
  misc_feature 1..17: note = Description of Artificial Sequence: Syntheticoligonucleotide
  source 1..17: mol_type = other DNA
tgatggatgttgcctaa

SEQ ID NO: 188 - DNA, 17 bp, synthetic construct
  misc_feature 1..17: note = Description of Artificial Sequence: Syntheticoligonucleotide
  source 1..17: mol_type = other DNA
gatggatgttgcctaat

SEQ ID NO: 189 - DNA, 17 bp, synthetic construct
  misc_feature 1..17: note = Description of Artificial Sequence: Syntheticoligonucleotide
  source 1..17: mol_type = other DNA
ttttacttccacctggc

SEQ ID NO: 190 - DNA, 17 bp, synthetic construct
  misc_feature 1..17: note = Description of Artificial Sequence: Syntheticoligonucleotide
  source 1..17: mol_type = other DNA
attttacttccacctgg

SEQ ID NO: 191 - DNA, 17 bp, synthetic construct
  misc_feature 1..17: note = Description of Artificial Sequence: Syntheticoligonucleotide
  source 1..17: mol_type = other DNA
gattttacttccacctg

SEQ ID NO: 192 - DNA, 17 bp, synthetic construct
  misc_feature 1..17: note = Description of Artificial Sequence: Syntheticoligonucleotide
  source 1..17: mol_type = other DNA
agattttacttccacct

SEQ ID NO: 193 - DNA, 17 bp, synthetic construct
  misc_feature 1..17: note = Description of Artificial Sequence: Syntheticoligonucleotide
  source 1..17: mol_type = other DNA
atttcctccgccaggga

SEQ ID NO: 194 - DNA, 17 bp, synthetic construct
  misc_feature 1..17: note = Description of Artificial Sequence: Syntheticoligonucleotide
  source 1..17: mol_type = other DNA
tttcctccgccagggac

SEQ ID NO: 195 - DNA, 17 bp, synthetic construct
  misc_feature 1..17: note = Description of Artificial Sequence: Syntheticoligonucleotide
  source 1..17: mol_type = other DNA
ttcctccgccagggacg

SEQ ID NO: 196 - DNA, 17 bp, synthetic construct
  misc_feature 1..17: note = Description of Artificial Sequence: Syntheticoligonucleotide
  source 1..17: mol_type = other DNA
tcctccgccagggacgt

SEQ ID NO: 197 - DNA, 17 bp, synthetic construct
  misc_feature 1..17: note = Description of Artificial Sequence: Syntheticoligonucleotide
  source 1..17: mol_type = other DNA
aaggtcagcttgtgggt

SEQ ID NO: 198 - DNA, 17 bp, synthetic construct
  misc_feature 1..17: note = Description of Artificial Sequence: Syntheticoligonucleotide
  source 1..17: mol_type = other DNA
gaaggtcagcttgtggg

SEQ ID NO: 199 - DNA, 17 bp, synthetic construct
  misc_feature 1..17: note = Description of Artificial Sequence: Syntheticoligonucleotide
  source 1..17: mol_type = other DNA
ggaaggtcagcttgtgg

SEQ ID NO: 200 - DNA, 17 bp, synthetic construct
  misc_feature 1..17: note = Description of Artificial Sequence: Syntheticoligonucleotide
  source 1..17: mol_type = other DNA
cggaaggtcagcttgtg

SEQ ID NO: 201 - DNA, 17 bp, synthetic construct
  misc_feature 1..17: note = Description of Artificial Sequence: Syntheticoligonucleotide
  source 1..17: mol_type = other DNA
accctgcttggccaggg

SEQ ID NO: 202 - DNA, 17 bp, synthetic construct
  misc_feature 1..17: note = Description of Artificial Sequence: Syntheticoligonucleotide
  source 1..17: mol_type = other DNA
ccctgcttggccaggga

SEQ ID NO: 203 - DNA, 17 bp, synthetic construct
  misc_feature 1..17: note = Description of Artificial Sequence: Syntheticoligonucleotide
  source 1..17: mol_type = other DNA
cctgcttggccagggag

SEQ ID NO: 204 - DNA, 17 bp, synthetic construct
  misc_feature 1..17: note = Description of Artificial Sequence: Syntheticoligonucleotide
  source 1..17: mol_type = other DNA
ctgcttggccagggagg

SEQ ID NO: 205 - DNA, 20 bp, synthetic construct
  misc_feature 1..20: note = Description of Artificial Sequence: Syntheticoligonucleotide
  source 1..20: mol_type = other DNA
ccgtacgtcccagcgtgatc

SEQ ID NO: 206 - DNA, 20 bp, synthetic construct
  misc_feature 1..20: note = Description of Artificial Sequence: Syntheticoligonucleotide
  source 1..20: mol_type = other DNA
ggctcagccatcctggttca

SEQ ID NO: 207 - DNA, 20 bp, synthetic construct
  misc_feature 1..20: note = Description of Artificial Sequence: Syntheticoligonucleotide
  source 1..20: mol_type = other DNA
cccgtacgtcccagcgtgat

SEQ ID NO: 208 - DNA, 20 bp, synthetic construct
  misc_feature 1..20: note = Description of Artificial Sequence: Syntheticoligonucleotide
  source 1..20: mol_type = other DNA
ggttgacatcgtctgcctgt

SEQ ID NO: 209 - DNA, 20 bp, synthetic construct
  misc_feature 1..20: note = Description of Artificial Sequence: Syntheticoligonucleotide
  source 1..20: mol_type = other DNA
gggctcagccatcctggttc

SEQ ID NO: 210 - DNA, 20 bp, synthetic construct
  misc_feature 1..20: note = Description of Artificial Sequence: Syntheticoligonucleotide
  source 1..20: mol_type = other DNA
ggccagcgtccgtgtcaccc

SEQ ID NO: 211 - DNA, 20 bp, synthetic construct
  misc_feature 1..20: note = Description of Artificial Sequence: Syntheticoligonucleotide
  source 1..20: mol_type = other DNA
ggctctcccagcggcaagga

SEQ ID NO: 212 - DNA, 20 bp, synthetic construct
  misc_feature 1..20: note = Description of Artificial Sequence: Syntheticoligonucleotide
  source 1..20: mol_type = other DNA
ccctcttggtcttggtgcat

SEQ ID NO: 213 - DNA, 20 bp, synthetic construct
  misc_feature 1..20: note = Description of Artificial Sequence: Syntheticoligonucleotide
  source 1..20: mol_type = other DNA
cgggacctgcctcccagacc

SEQ ID NO: 214 - DNA, 20 bp, synthetic construct
  misc_feature 1..20: note = Description of Artificial Sequence: Syntheticoligonucleotide
  source 1..20: mol_type = other DNA
gctggtctctgttgggtccc

SEQ ID NO: 215 - DNA, 20 bp, synthetic construct
  misc_feature 1..20: note = Description of Artificial Sequence: Syntheticoligonucleotide
  source 1..20: mol_type = other DNA
gggctctctccatgtcaaca

SEQ ID NO: 216 - DNA, 20 bp, synthetic construct
  misc_feature 1..20: note = Description of Artificial Sequence: Syntheticoligonucleotide
  source 1..20: mol_type = other DNA
ggtctctgttgggtcccagg

SEQ ID NO: 217 - DNA, 20 bp, synthetic construct
  misc_feature 1..20: note = Description of Artificial Sequence: Syntheticoligonucleotide
  source 1..20: mol_type = other DNA
gggacctgcctcccagaccc

SEQ ID NO: 218 - DNA, 20 bp, synthetic construct
  misc_feature 1..20: note = Description of Artificial Sequence: Syntheticoligonucleotide
  source 1..20: mol_type = other DNA
cccaacccgtacgtcccagc

SEQ ID NO: 219 - DNA, 20 bp, synthetic construct
  misc_feature 1..20: note = Description of Artificial Sequence: Syntheticoligonucleotide
  source 1..20: mol_type = other DNA
gcttcgtcttccaggctggg

SEQ ID NO: 220 - DNA, 20 bp, synthetic construct
  misc_feature 1..20: note = Description of Artificial Sequence: Syntheticoligonucleotide
  source 1..20: mol_type = other DNA
ccgtgtcaccctcttggtct

SEQ ID NO: 221 - DNA, 20 bp, synthetic construct
  misc_feature 1..20: note = Description of Artificial Sequence: Syntheticoligonucleotide
  source 1..20: mol_type = other DNA
cttggctctcccagcggcaa

SEQ ID NO: 222 - DNA, 20 bp, synthetic construct
  misc_feature 1..20: note = Description of Artificial Sequence: Syntheticoligonucleotide
  source 1..20: mol_type = other DNA
cggcctccttagctgctaga

SEQ ID NO: 223 - DNA, 20 bp, synthetic construct
  misc_feature 1..20: note = Description of Artificial Sequence: Syntheticoligonucleotide
  source 1..20: mol_type = other DNA
cagcgtccgtgtcaccctct

SEQ ID NO: 224 - DNA, 20 bp, synthetic construct
  misc_feature 1..20: note = Description of Artificial Sequence: Syntheticoligonucleotide
  source 1..20: mol_type = other DNA
gctcagccatcctggttcaa

SEQ ID NO: 225 - DNA, 20 bp, synthetic construct
  misc_feature 1..20: note = Description of Artificial Sequence: Syntheticoligonucleotide
  source 1..20: mol_type = other DNA
cctggactttgccttccctt

SEQ ID NO: 226 - DNA, 20 bp, synthetic construct
  misc_feature 1..20: note = Description of Artificial Sequence: Syntheticoligonucleotide
  source 1..20: mol_type = other DNA
gtcccactcttgtgcctgga

SEQ ID NO: 227 - DNA, 20 bp, synthetic construct
  misc_feature 1..20: note = Description of Artificial Sequence: Syntheticoligonucleotide
  source 1..20: mol_type = other DNA
acctggccacctcctggttt

SEQ ID NO: 228 - DNA, 20 bp, synthetic construct
  misc_feature 1..20: note = Description of Artificial Sequence: Syntheticoligonucleotide
  source 1..20: mol_type = other DNA
ttggctttggcgttctcgcg

SEQ ID NO: 229 - DNA, 20 bp, synthetic construct
  misc_feature 1..20: note = Description of Artificial Sequence: Syntheticoligonucleotide
  source 1..20: mol_type = other DNA
cgcttccagtcccgtctttg

SEQ ID NO: 230 - DNA, 20 bp, synthetic construct
  misc_feature 1..20: note = Description of Artificial Sequence: Syntheticoligonucleotide
  source 1..20: mol_type = other DNA
ggtgatcacctctgccctcg

SEQ ID NO: 231 - DNA, 20 bp, synthetic construct
  misc_feature 1..20: note = Description of Artificial Sequence: Syntheticoligonucleotide
  source 1..20: mol_type = other DNA
ggtacctcctgcaaccaacc

SEQ ID NO: 232 - DNA, 20 bp, synthetic construct
  misc_feature 1..20: note = Description of Artificial Sequence: Syntheticoligonucleotide
  source 1..20: mol_type = other DNA
cacgtggcttcctctcccac

SEQ ID NO: 233 - DNA, 20 bp, synthetic construct
  misc_feature 1..20: note = Description of Artificial Sequence: Syntheticoligonucleotide
  source 1..20: mol_type = other DNA
gcgtccgtgtcaccctcttg

SEQ ID NO: 234 - DNA, 20 bp, synthetic construct
  misc_feature 1..20: note = Description of Artificial Sequence: Syntheticoligonucleotide
  source 1..20: mol_type = other DNA
caccctcttggtcttggtgc

SEQ ID NO: 235 - DNA, 20 bp, synthetic construct
  misc_feature 1..20: note = Description of Artificial Sequence: Syntheticoligonucleotide
  source 1..20: mol_type = other DNA
gtcccagcgtgatcttccat

SEQ ID NO: 236 - DNA, 20 bp, synthetic construct
  misc_feature 1..20: note = Description of Artificial Sequence: Syntheticoligonucleotide
  source 1..20: mol_type = other DNA
gccagcactgatcaccctaa

SEQ ID NO: 237 - DNA, 20 bp, synthetic construct
  misc_feature 1..20: note = Description of Artificial Sequence: Syntheticoligonucleotide
  source 1..20: mol_type = other DNA
tggtctctgttgggtcccag

SEQ ID NO: 238 - DNA, 20 bp, synthetic construct
  misc_feature 1..20: note = Description of Artificial Sequence: Syntheticoligonucleotide
  source 1..20: mol_type = other DNA
ccgcctcccggctggtgctt

SEQ ID NO: 239 - DNA, 20 bp, synthetic construct
  misc_feature 1..20: note = Description of Artificial Sequence: Syntheticoligonucleotide
  source 1..20: mol_type = other DNA
ggccacacgagtcccagtgt

SEQ ID NO: 240 - DNA, 20 bp, synthetic construct
  misc_feature 1..20: note = Description of Artificial Sequence: Syntheticoligonucleotide
  source 1..20: mol_type = other DNA
gtccctcagggttgccttta

SEQ ID NO: 241 - DNA, 20 bp, synthetic construct
  misc_feature 1..20: note = Description of Artificial Sequence: Syntheticoligonucleotide
  source 1..20: mol_type = other DNA
ggaccactgccaccttcttg

SEQ ID NO: 242 - DNA, 20 bp, synthetic construct
  misc_feature 1..20: note = Description of Artificial Sequence: Syntheticoligonucleotide
  source 1..20: mol_type = other DNA
cacctggccacctcctggtt

SEQ ID NO: 243 - DNA, 20 bp, synthetic construct
  misc_feature 1..20: note = Description of Artificial Sequence: Syntheticoligonucleotide
  source 1..20: mol_type = other DNA
cccgcctcccggctggtgct

SEQ ID NO: 244 - DNA, 20 bp, synthetic construct
  misc_feature 1..20: note = Description of Artificial Sequence: Syntheticoligonucleotide
  source 1..20: mol_type = other DNA
ggtgccttgcccttccatcc

SEQ ID NO: 245 - DNA, 20 bp, synthetic construct
  misc_feature 1..20: note = Description of Artificial Sequence: Syntheticoligonucleotide
  source 1..20: mol_type = other DNA
cccgtcacactcacacaagg

SEQ ID NO: 246 - DNA, 20 bp, synthetic construct
  misc_feature 1..20: note = Description of Artificial Sequence: Syntheticoligonucleotide
  source 1..20: mol_type = other DNA
cccaatccctgctgtggtcg

SEQ ID NO: 247 - DNA, 20 bp, synthetic construct
  misc_feature 1..20: note = Description of Artificial Sequence: Syntheticoligonucleotide
  source 1..20: mol_type = other DNA
gggtcccactcttgtgcctg

SEQ ID NO: 248 - DNA, 20 bp, synthetic construct
  misc_feature 1..20: note = Description of Artificial Sequence: Syntheticoligonucleotide
  source 1..20: mol_type = other DNA
gcttccagtcccgtctttgc

SEQ ID NO: 249 - DNA, 20 bp, synthetic construct
  misc_feature 1..20: note = Description of Artificial Sequence: Syntheticoligonucleotide
  source 1..20: mol_type = other DNA
cccttctcccacaggctgcc

SEQ ID NO: 250 - DNA, 20 bp, synthetic construct
  misc_feature 1..20: note = Description of Artificial Sequence: Syntheticoligonucleotide
  source 1..20: mol_type = other DNA
ctggtgccaccactgacaac

SEQ ID NO: 251 - DNA, 20 bp, synthetic construct
  misc_feature 1..20: note = Description of Artificial Sequence: Syntheticoligonucleotide
  source 1..20: mol_type = other DNA
gccactgcctctgtgacacc

SEQ ID NO: 252 - DNA, 20 bp, synthetic construct
  misc_feature 1..20: note = Description of Artificial Sequence: Syntheticoligonucleotide
  source 1..20: mol_type = other DNA
gtgccaccactgacaaccaa

SEQ ID NO: 253 - DNA, 20 bp, synthetic construct
  misc_feature 1..20: note = Description of Artificial Sequence: Syntheticoligonucleotide
  source 1..20: mol_type = other DNA
cttgcccttccatcctggtg

SEQ ID NO: 254 - DNA, 20 bp, synthetic construct
  misc_feature 1..20: note = Description of Artificial Sequence: Syntheticoligonucleotide
  source 1..20: mol_type = other DNA
gcctggactttgccttccct

SEQ ID NO: 255 - DNA, 20 bp, synthetic construct
  misc_feature 1..20: note = Description of Artificial Sequence: Syntheticoligonucleotide
  source 1..20: mol_type = other DNA
gcctctaactccgtggctgc

SEQ ID NO: 256 - DNA, 20 bp, synthetic construct
  misc_feature 1..20: note = Description of Artificial Sequence: Syntheticoligonucleotide
  source 1..20: mol_type = other DNA
gatcccagagccttccgtat

SEQ ID NO: 257 - DNA, 20 bp, synthetic construct
  misc_feature 1..20: note = Description of Artificial Sequence: Syntheticoligonucleotide
  source 1..20: mol_type = other DNA
catcctcgcgccgcaagcca

SEQ ID NO: 258 - DNA, 20 bp, synthetic construct
  misc_feature 1..20: note = Description of Artificial Sequence: Syntheticoligonucleotide
  source 1..20: mol_type = other DNA
gcctcccggctggtgcttca

SEQ ID NO: 259 - DNA, 20 bp, synthetic construct
  misc_feature 1..20: note = Description of Artificial Sequence: Syntheticoligonucleotide
  source 1..20: mol_type = other DNA
gtgcctggactttgccttcc

SEQ ID NO: 260 - DNA, 20 bp, synthetic construct
  misc_feature 1..20: note = Description of Artificial Sequence: Syntheticoligonucleotide
  source 1..20: mol_type = other DNA
ctgccactgcctctgtgaca

SEQ ID NO: 261 - DNA, 20 bp, synthetic construct
  misc_feature 1..20: note = Description of Artificial Sequence: Syntheticoligonucleotide
  source 1..20: mol_type = other DNA
cctggccacctcctggttta

SEQ ID NO: 262 - DNA, 20 bp, synthetic construct
  misc_feature 1..20: note = Description of Artificial Sequence: Syntheticoligonucleotide
  source 1..20: mol_type = other DNA
gggtgccttgcccttccatc

SEQ ID NO: 263 - DNA, 20 bp, synthetic construct
  misc_feature 1..20: note = Description of Artificial Sequence: Syntheticoligonucleotide
  source 1..20: mol_type = other DNA
ccactcccacttcttgtgct

SEQ ID NO: 264 - DNA, 20 bp, synthetic construct
  misc_feature 1..20: note = Description of Artificial Sequence: Syntheticoligonucleotide
  source 1..20: mol_type = other DNA
gtgcttcaggccttcgtcac

SEQ ID NO: 265 - DNA, 20 bp, synthetic construct
  misc_feature 1..20: note = Description of Artificial Sequence: Syntheticoligonucleotide
  source 1..20: mol_type = other DNA
ctgccagcttgccttctctt

SEQ ID NO: 266 - DNA, 20 bp, synthetic construct
  misc_feature 1..20: note = Description of Artificial Sequence: Syntheticoligonucleotide
  source 1..20: mol_type = other DNA
ctcccggctggtgcttcagg

SEQ ID NO: 267 - DNA, 20 bp, synthetic construct
  misc_feature 1..20: note = Description of Artificial Sequence: Syntheticoligonucleotide
  source 1..20: mol_type = other DNA
cgcctcccggctggtgcttc

SEQ ID NO: 268 - DNA, 20 bp, synthetic construct
  misc_feature 1..20: note = Description of Artificial Sequence: Syntheticoligonucleotide
  source 1..20: mol_type = other DNA
ctggccacctcctggtttat

SEQ ID NO: 269 - DNA, 20 bp, synthetic construct
  misc_feature 1..20: note = Description of Artificial Sequence: Syntheticoligonucleotide
  source 1..20: mol_type = other DNA
ggccacctcctggtttatga

SEQ ID NO: 270 - DNA, 20 bp, synthetic construct
  misc_feature 1..20: note = Description of Artificial Sequence: Syntheticoligonucleotide
  source 1..20: mol_type = other DNA
ccatcctggtgccaccactg

SEQ ID NO: 271 - DNA, 20 bp, synthetic construct
  misc_feature 1..20: note = Description of Artificial Sequence: Syntheticoligonucleotide
  source 1..20: mol_type = other DNA
cctgccagcttgccttctct

SEQ ID NO: 272 - DNA, 20 bp, synthetic construct
  misc_feature 1..20: note = Description of Artificial Sequence: Syntheticoligonucleotide
  source 1..20: mol_type = other DNA
aatccctgctgtggtcgcag

SEQ ID NO: 273 - DNA, 20 bp, synthetic construct
  misc_feature 1..20: note = Description of Artificial Sequence: Syntheticoligonucleotide
  source 1..20: mol_type = other DNA
gccaccactgacaaccaaga

SEQ ID NO: 274 - DNA, 19 bp, synthetic construct
  misc_feature 1..19: note = Description of Artificial Sequence: Syntheticoligonucleotide
  source 1..19: mol_type = other DNA
cttgtcggccatgatatag

SEQ ID NO: 275 - DNA, 20 bp, synthetic construct
  misc_feature 1..20: note = Description of Artificial Sequence: Syntheticoligonucleotide
  source 1..20: mol_type = other DNA
taagcagtgggttctctagt

SEQ ID NO: 276 - DNA, 20 bp, synthetic construct
  misc_feature 1..20: note = Description of Artificial Sequence: Syntheticoligonucleotide
  source 1..20: mol_type = other DNA
cctcccggctggtgcttcag

SEQ ID NO: 277 - DNA, 20 bp, synthetic construct
  misc_feature 1..20: note = Description of Artificial Sequence: Syntheticoligonucleotide
  source 1..20: mol_type = other DNA
ctcctgccagcttgccttct

SEQ ID NO: 278 - DNA, 20 bp, synthetic construct
  misc_feature 1..20: note = Description of Artificial Sequence: Syntheticoligonucleotide
  source 1..20: mol_type = other DNA
cttctcctccggccactagt

SEQ ID NO: 279 - DNA, 20 bp, synthetic construct
  misc_feature 1..20: note = Description of Artificial Sequence: Syntheticoligonucleotide
  source 1..20: mol_type = other DNA
ctcctccggccactagtggg

SEQ ID NO: 280 - DNA, 20 bp, synthetic construct
  misc_feature 1..20: note = Description of Artificial Sequence: Syntheticoligonucleotide
  source 1..20: mol_type = other DNA
ccttctcctccggccactag

SEQ ID NO: 281 - DNA, 20 bp, synthetic construct
  misc_feature 1..20: note = Description of Artificial Sequence: Syntheticoligonucleotide
  source 1..20: mol_type = other DNA
gagccttctcctccggccac

SEQ ID NO: 282 - DNA, 20 bp, synthetic construct
  misc_feature 1..20: note = Description of Artificial Sequence: Syntheticoligonucleotide
  source 1..20: mol_type = other DNA
gccttctcctccggccacta

SEQ ID NO: 283 - DNA, 20 bp, synthetic construct
  misc_feature 1..20: note = Description of Artificial Sequence: Syntheticoligonucleotide
  source 1..20: mol_type = other DNA
ccttacctgctagctggcgt

SEQ ID NO: 284 - DNA, 20 bp, synthetic construct
  misc_feature 1..20: note = Description of Artificial Sequence: Syntheticoligonucleotide
  source 1..20: mol_type = other DNA
ccgtacgtcccagcgtgatc

SEQ ID NO: 285 - DNA, 20 bp, synthetic construct
  misc_feature 1..20: note = Description of Artificial Sequence: Syntheticoligonucleotide
  modified_base 1..5: mod_base = OTHER; note = 2'-O-MOE modified nucleotide
  modified_base 7: mod_base = OTHER; note = 5-methylcytosine
  modified_base 10: mod_base = OTHER; note = 5-methylcytosine
  modified_base 13: mod_base = OTHER; note = 5-methylcytosine
  modified_base 16..17: mod_base = OTHER; note = 5-methylcytosine
  modified_base 16..20: mod_base = OTHER; note = 2'-O-MOE modified nucleotide
  source 1..20: mol_type = other DNA
ggttgacatcgtctgcctgt

SEQ ID NO: 286 - DNA, 20 bp, synthetic construct
  misc_feature 1..20: note = Description of Artificial Sequence: Syntheticoligonucleotide
  source 1..20: mol_type = other DNA
gggctcagccatcctggttc

SEQ ID NO: 287 - DNA, 20 bp, synthetic construct
  misc_feature 1..20: note = Description of Artificial Sequence: Syntheticoligonucleotide
  source 1..20: mol_type = other DNA
ggctctcccagcggcaagga

SEQ ID NO: 288 - DNA, 20 bp, synthetic construct
  misc_feature 1..20: note = Description of Artificial Sequence: Syntheticoligonucleotide
  source 1..20: mol_type = other DNA
ccctcttggtcttggtgcat

SEQ ID NO: 289 - DNA, 20 bp, synthetic construct
  misc_feature 1..20: note = Description of Artificial Sequence: Syntheticoligonucleotide
  source 1..20: mol_type = other DNA
gctggtctctgttgggtccc

SEQ ID NO: 290 - DNA, 20 bp, synthetic construct
  misc_feature 1..20: note = Description of Artificial Sequence: Syntheticoligonucleotide
  source 1..20: mol_type = other DNA
gtcccactcttgtgcctgga

SEQ ID NO: 291 - DNA, 20 bp, synthetic construct
  misc_feature 1..20: note = Description of Artificial Sequence: Syntheticoligonucleotide
  source 1..20: mol_type = other DNA
gggctctctccatgtcaaca

SEQ ID NO: 292 - DNA, 25 bp, synthetic construct
  misc_feature 1..25: note = Description of Artificial Sequence: Syntheticoligonucleotide
  source 1..25: mol_type = other DNA
acgctggcctgaaaggttagtggac

SEQ ID NO: 293 - DNA, 25 bp, synthetic construct
  misc_feature 1..25: note = Description of Artificial Sequence: Syntheticoligonucleotide
  source 1..25: mol_type = other DNA
aaaagccaaggtaagctgacgatgc

SEQ ID NO: 294 - DNA, 25 bp, synthetic construct
  misc_feature 1..25: note = Description of Artificial Sequence: Syntheticoligonucleotide
  source 1..25: mol_type = other DNA
ttttatattttatcagctcgcatgg

SEQ ID NO: 295 - DNA, 20 bp, synthetic construct
  misc_feature 1..20: note = Description of Artificial Sequence: Syntheticoligonucleotide
  source 1..20: mol_type = other DNA
acccacaagctgaccttccg

SEQ ID NO: 296 - DNA, 30 bp, synthetic construct
  misc_feature 1..30: note = Description of Artificial Sequence: Syntheticoligonucleotide
  source 1..30: mol_type = other DNA
accagctgaagaagcaggcattggagacac

SEQ ID NO: 297 - DNA, 25 bp, synthetic construct
  misc_feature 1..25: note = Description of Artificial Sequence: Syntheticoligonucleotide
  source 1..25: mol_type = other DNA
ctctcatctccaggtgcaaatagtc

SEQ ID NO: 298 - DNA, 20 bp, synthetic construct
  misc_feature 1..20: note = Description of Artificial Sequence: Syntheticoligonucleotide
  source 1..20: mol_type = other DNA
atagtctacaaaccagttga

SEQ ID NO: 299 - DNA, 20 bp, synthetic construct
  misc_feature 1..20: note = Description of Artificial Sequence: Syntheticoligonucleotide
  source 1..20: mol_type = other DNA
attaggcaacatccatcata

SEQ ID NO: 300 - DNA, 20 bp, synthetic construct
  misc_feature 1..20: note = Description of Artificial Sequence: Syntheticoligonucleotide
  source 1..20: mol_type = other DNA
gaaccaggatggctgagccc

SEQ ID NO: 301 - DNA, 18 bp, synthetic construct
  misc_feature 1..18: note = Description of Artificial Sequence: Syntheticoligonucleotide
  source 1..18: mol_type = other DNA
cgtccctggcggaggaaa

SEQ ID NO: 302 - DNA, 20 bp, synthetic construct
  misc_feature 1..20: note = Description of Artificial Sequence: Syntheticoligonucleotide
  source 1..20: mol_type = other DNA
tggtcagtaaaagcaaagac

SEQ ID NO: 303 - DNA, 20 bp, synthetic construct
  misc_feature 1..20: note = Description of Artificial Sequence: Syntheticoligonucleotide
  source 1..20: mol_type = other DNA
ctggaagcgatgacaaaaaa

SEQ ID NO: 304 - DNA, 140924 bp, Homo sapiens
  source 1..140924: mol_type = genomic DNA
gggattacaggcgtgagccaccacacccagcccagaatgtttattagaatgcacaattaataccagaggcagtggggaaggaaggactgagcagaggaggaagttgagttgtgattcaacccaacaactgcctggctggcatggggagctctggagttaaatagggccatcagactttcccagtgtggggccaacatgactgggtctttatacccccacctctgtcagtcactcaacgtggtctccctgcaacaaggtgactcttgcagccgagacaatccctgaagggacagaggctgaagcctgtctgccaacagcactcccagtggctggaacaagtccttccctataggggaatctgggcggcacacctccatctccatgtccatcacatacgatatcacagacatttaaatattttgataactgtacataagagtttcctttataatcttatagatcttattttatgcatttgaaaatattcttctgagacagggcttttatcatattgccatagggtgccacgatataaaaaaggttaaatactctctgattcagaagtatccaatgatgacttctctctcatgcatttaattgaaaatctggtttttctccttctctgctagttctctacctctctccccacctcccacatcatagcctattcacatatgtctgaatctcatgatagacaagttcaggttcttttcccaggttctttttaccacatccccccacccccacataaaaagtatatatggcacagcctaggttccacccaaatcctttctcctcttcttcctgggcccacaactctcctacatacattggtataccttgcgcttagggatggccatgtgactaagttctaacagtggaacatgatcagatgccacttccagcctctaagacagccagtgtgtttcctccataagctccttctcttcctcccaactggagactctaaatgatgaccctgcctcaagcaagcaaacaacaagtccctcaggggtggtgtaggctgcaaatggaaggagcttgagtcccaaaccttccacggagaaggctggctaccaacctggatcactcacccaagactgctcgaagagttggtttgaaccattgtgttttggggtctatttattacaacagtttagcttgctttgtgaatagatttagtggcagagcctccaaattctatagatacattgatctcagtcctaaccgcatctggaacaccattaaataaaggaattgcaaacccagagaaggtaatgaatttgtctaaggtcatacaagatggctaggatcaggacccaactctccagttttctttcttctctgctattctgccttctgtgatcctacataagtgggcatgattgtataacatatgcggccatgagatttctctttcagcaagagaaagggacaggaagaaagagagggaatgcattttcttggcctgaattagtgtgagccattagttacctacattgactaaattatctggaatgaacattcaactctacatcacatatagttaaaatgacagatctgcttaagattgtttctagcatacgttatttcaatttaggcaaatgtgaccattcagtgtgaggggaccatactgtcattaggtccctgtcagttctcaattatactgttatcttagagggggaaaaatgtgaaatttgaatgtagacgagtgttgatttgactgctacagtttattttacgtatagaaataaaataatgtgtagcaaaagcattattacaaagatgataatgaaataactagtatttataatagtataatagtatagtatttataatagtatgatagtttaatgactatttgtcagatgttgtgtaagaaactttatacacacacacacacacacctcatttaattcctgtatcaatcaggatacaggacgctgtggtaacaactcctcaaatctcggtggcttgcacaacaaatgcttatttcttttttttttttgacaccaagtcttgctctgtaacaggctggagtgcaatggtgcaatctcggctcactgcagcctctgcctcctgggttcaagcgattctcctgcctcagtctctcgagtagctgggaacacaggcacgcgccaccacatctggctaatttttgtgattttagtagagatgggatttcaccatgttgctcaggctggccttgaactcctgacctcaagcgatccacccacctcagcctcccaaagtgctgggattacaggcatgagccactgcgcccagccccaaatgtttatttcttgctcatgtgacatgtacttcctcgagtttttccttcctgagatctaagctgaaggaacagctctctggagccacgccattctggtggcggaaaggaagagtaaaagtggtagaaccttgcaatgctcttgaagcgcctatttggaatgtctacatcatgtaaatggtaatggacaagtatgtataatccccacaccaaaaaaaggggacactattggggacaataaccacatttcaatgctgcaagacggatattgactgcacccccttcccactttcagaaagaagaagagtaattttgctgaactccttctagagactggaaatgtcccttccagttggggtgattagggaaggctttggtaaaatttgagctagagtttgaaggttaggtagactactggtgggtgaagaaagaacaaggacctttgtaggcaaaggaaaacctcagaattacagaggtggaaaaagagttctagtcaagccacttcagctggctacagagtaggtgggaaagaaaatgggaggacaagggctcagatgatggggggttggggcattggggggacacttgaaagctaaactaaggggttgaacttaatttaggaggcagttagaagcttttacatatttttgagcaagagagtgacataattaaaatgatctgggccaggtgtggtggctcacacctgtaatcccagcactttgggaggctgaggagcttgggtcacctgaggtcaggagatcgagaccagcctggccaacatggtgaaatcccgtcctactaaaaatacaaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcttgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccacaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatttttttttatatatataatatatattaaaatataatatatatatttatataatataatatataaatatattatatattatatatttttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatatatttttatatatataatatgtataatatataatatatataaaaacatatataatatatattatatattatatatatattatatatattatatatattaaatatattttatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaatttaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagatttttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgatttttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccctccttccctccttccctctttccctccttccccccttccacccttccccccttccccccttccctccttccttccttccctccttccttccttccttcctgccttccttccttcctgccttccttccttcctgccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtacaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactccttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaaaaaaaatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttatgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctacaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattccgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgagcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacatgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatctcctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggtgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagcgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcccggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatcccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaagaatcaaatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcgtcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagaaagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcgtggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcgttgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaaaaaaggaaaaaaaaaactcatgcctgtaatcccagcactttggggaccggggtgggcagatcacgaggtcaggagatcaagactatcctagccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctccccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcggaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatctttgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgccgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccaagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagagaccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaacacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtgttatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcggtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcatttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccactaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcccggctaattttttttttttttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagccgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtgggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagcaggcaagaaccaatccaggctctaagcaaattatgtatctcactgaccccaccaaacctcagaaaaatttaacagtgagaagcaaaatctcctttaaagagcaacttagaacagatagaaaatatcatacagctgacttcactagagagaaagtgcatcaactgctttcactcaacaaaaagaaaaaagagatgatcaatgcagatcccctctcctcctggcagcccttaccctcagtgaaaagccaccaccattctctctctggtggccatcagatcaacctgcggcgttcccacaagacagaatggagattttccaaggtatagagcaagtcagagtaccccaaagaacggcggcagagagccagctccgaaactgccaacactaccatgcatacacagttcagtaagtcaagaaaggcctggtacacagcattctgtaacttttttttttatttttttcaatttttccttcttttttttttttaagcactagtctgtgctttgcgaacagaatcaagacattaacaaagatcagcttctctgaagaaaagcatttctatagaacaaagacagctacatgtttcgctgccattacacagctccaaagcaggaaaagaaaatatttacaaaatacaaggttttttttttccattttttgtttttgttttttttttcaatgctaaaagggttattcagaattttcaaccttataaatagaagaagcactttatgcatagggatatggtgcattattgtatttttttttaaagaaacaatgacaaaccctttaacttgcaaacagaaaaaaaaatcactaatgttgaaaattgtgaaaaaaccccaaccattaa

SEQ ID NO: 305 - DNA, 6816 bp, Homo sapiens
  source 1..6816: mol_type = genomic DNA
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 306 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 307 - DNA, 20 bp, synthetic construct
  misc_feature 1..20: note = Description of Artificial Sequence: Syntheticoligonucleotide
  source 1..20: mol_type = other DNA
gcccttctggcctggagggg

SEQ ID NO: 308 - DNA, 20 bp, synthetic construct
  misc_feature 1..20: note = Description of Artificial Sequence: Syntheticoligonucleotide
  source 1..20: mol_type = other DNA
tggcccttctggcctggagg

SEQ ID NO: 309 - DNA, 20 bp, synthetic construct
  misc_feature 1..20: note = Description of Artificial Sequence: Syntheticoligonucleotide
  source 1..20: mol_type = other DNA
gctggtgcttcaggttctca

SEQ ID NO: 310 - DNA, 20 bp, synthetic construct
  misc_feature 1..20: note = Description of Artificial Sequence: Syntheticoligonucleotide
  source 1..20: mol_type = other DNA
tcaggtcaactggtttgtag

SEQ ID NO: 311 - DNA, 20 bp, synthetic construct
  misc_feature 1..20: note = Description of Artificial Sequence: Syntheticoligonucleotide
  source 1..20: mol_type = other DNA
tgctcaggtcaactggtttg

SEQ ID NO: 312 - DNA, 20 bp, synthetic construct
  misc_feature 1..20: note = Description of Artificial Sequence: Syntheticoligonucleotide
  source 1..20: mol_type = other DNA
ttgctcaggtcaactggttt

SEQ ID NO: 313 - DNA, 20 bp, synthetic construct
  misc_feature 1..20: note = Description of Artificial Sequence: Syntheticoligonucleotide
  source 1..20: mol_type = other DNA
ccttgctcaggtcaactggt

SEQ ID NO: 314 - DNA, 20 bp, synthetic construct
  misc_feature 1..20: note = Description of Artificial Sequence: Syntheticoligonucleotide
  source 1..20: mol_type = other DNA
ccctcttctacatggagggg

SEQ ID NO: 315 - DNA, 20 bp, synthetic construct
  misc_feature 1..20: note = Description of Artificial Sequence: Syntheticoligonucleotide
  source 1..20: mol_type = other DNA
ttctccctcttctacatgga

SEQ ID NO: 316 - DNA, 20 bp, synthetic construct
  misc_feature 1..20: note = Description of Artificial Sequence: Syntheticoligonucleotide
  source 1..20: mol_type = other DNA
cttctccctcttctacatgg

SEQ ID NO: 317 - DNA, 20 bp, synthetic construct
  misc_feature 1..20: note = Description of Artificial Sequence: Syntheticoligonucleotide
  source 1..20: mol_type = other DNA
ccttctccctcttctacatg

SEQ ID NO: 318 - DNA, 20 bp, synthetic construct
  misc_feature 1..20: note = Description of Artificial Sequence: Syntheticoligonucleotide
  source 1..20: mol_type = other DNA
caaatcctttgttgctgcca

SEQ ID NO: 319 - DNA, 20 bp, synthetic construct
  misc_feature 1..20: note = Description of Artificial Sequence: Syntheticoligonucleotide
  source 1..20: mol_type = other DNA
tcaaatcctttgttgctgcc

SEQ ID NO: 320 - DNA, 20 bp, synthetic construct
  misc_feature 1..20: note = Description of Artificial Sequence: Syntheticoligonucleotide
  source 1..20: mol_type = other DNA
tggctccacgaacacaccaa

SEQ ID NO: 321 - DNA, 20 bp, synthetic construct
  misc_feature 1..20: note = Description of Artificial Sequence: Syntheticoligonucleotide
  source 1..20: mol_type = other DNA
gtggctccacgaacacacca

SEQ ID NO: 322 - DNA, 20 bp, synthetic construct
  misc_feature 1..20: note = Description of Artificial Sequence: Syntheticoligonucleotide
  source 1..20: mol_type = other DNA
tgtggctccacgaacacacc

SEQ ID NO: 323 - DNA, 20 bp, synthetic construct
  misc_feature 1..20: note = Description of Artificial Sequence: Syntheticoligonucleotide
  source 1..20: mol_type = other DNA
ctgtggctccacgaacacac

SEQ ID NO: 324 - DNA, 20 bp, synthetic construct
  misc_feature 1..20: note = Description of Artificial Sequence: Syntheticoligonucleotide
  source 1..20: mol_type = other DNA
cctgtggctccacgaacaca

SEQ ID NO: 325 - DNA, 20 bp, synthetic construct
  misc_feature 1..20: note = Description of Artificial Sequence: Syntheticoligonucleotide
  source 1..20: mol_type = other DNA
gcctgtggctccacgaacac

SEQ ID NO: 326 - DNA, 20 bp, synthetic construct
  misc_feature 1..20: note = Description of Artificial Sequence: Syntheticoligonucleotide
  source 1..20: mol_type = other DNA
tgcctgtggctccacgaaca

SEQ ID NO: 327 - DNA, 20 bp, synthetic construct
  misc_feature 1..20: note = Description of Artificial Sequence: Syntheticoligonucleotide
  source 1..20: mol_type = other DNA
ctgcctgtggctccacgaac

SEQ ID NO: 328 - DNA, 20 bp, synthetic construct
  misc_feature 1..20: note = Description of Artificial Sequence: Syntheticoligonucleotide
  source 1..20: mol_type = other DNA
tctgcctgtggctccacgaa

SEQ ID NO: 329 - DNA, 20 bp, synthetic construct
  misc_feature 1..20: note = Description of Artificial Sequence: Syntheticoligonucleotide
  source 1..20: mol_type = other DNA
gtctgcctgtggctccacga

SEQ ID NO: 330 - DNA, 20 bp, synthetic construct
  misc_feature 1..20: note = Description of Artificial Sequence: Syntheticoligonucleotide
  source 1..20: mol_type = other DNA
cgtctgcctgtggctccacg

SEQ ID NO: 331 - DNA, 20 bp, synthetic construct
  misc_feature 1..20: note = Description of Artificial Sequence: Syntheticoligonucleotide
  source 1..20: mol_type = other DNA
tcgtctgcctgtggctccac

SEQ ID NO: 332 - DNA, 20 bp, synthetic construct
  misc_feature 1..20: note = Description of Artificial Sequence: Syntheticoligonucleotide
  source 1..20: mol_type = other DNA
atcgtctgcctgtggctcca

SEQ ID NO: 333 - DNA, 20 bp, synthetic construct
  misc_feature 1..20: note = Description of Artificial Sequence: Syntheticoligonucleotide
  source 1..20: mol_type = other DNA
catcgtctgcctgtggctcc

SEQ ID NO: 334 - DNA, 20 bp, synthetic construct
  misc_feature 1..20: note = Description of Artificial Sequence: Syntheticoligonucleotide
  source 1..20: mol_type = other DNA
acatcgtctgcctgtggctc

SEQ ID NO: 335 - DNA, 20 bp, synthetic construct
  misc_feature 1..20: note = Description of Artificial Sequence: Syntheticoligonucleotide
  source 1..20: mol_type = other DNA
gacatcgtctgcctgtggct

SEQ ID NO: 336 - DNA, 20 bp, synthetic construct
  misc_feature 1..20: note = Description of Artificial Sequence: Syntheticoligonucleotide
  source 1..20: mol_type = other DNA
tgacatcgtctgcctgtggc

SEQ ID NO: 337 - DNA, 20 bp, synthetic construct
  misc_feature 1..20: note = Description of Artificial Sequence: Syntheticoligonucleotide
  source 1..20: mol_type = other DNA
ttgacatcgtctgcctgtgg

SEQ ID NO: 338 - DNA, 20 bp, synthetic construct
  misc_feature 1..20: note = Description of Artificial Sequence: Syntheticoligonucleotide
  source 1..20: mol_type = other DNA
gttgacatcgtctgcctgtg

SEQ ID NO: 339 - DNA, 20 bp, synthetic construct
  misc_feature 1..20: note = Description of Artificial Sequence: Syntheticoligonucleotide
  source 1..20: mol_type = other DNA
aggttgacatcgtctgcctg

SEQ ID NO: 340 - DNA, 20 bp, synthetic construct
  misc_feature 1..20: note = Description of Artificial Sequence: Syntheticoligonucleotide
  source 1..20: mol_type = other DNA
aaggttgacatcgtctgcct

SEQ ID NO: 341 - DNA, 20 bp, synthetic construct
  misc_feature 1..20: note = Description of Artificial Sequence: Syntheticoligonucleotide
  source 1..20: mol_type = other DNA
caaggttgacatcgtctgcc

SEQ ID NO: 342 - DNA, 20 bp, synthetic construct
  misc_feature 1..20: note = Description of Artificial Sequence: Syntheticoligonucleotide
  source 1..20: mol_type = other DNA
acaaggttgacatcgtctgc

SEQ ID NO: 343 - DNA, 20 bp, synthetic construct
  misc_feature 1..20: note = Description of Artificial Sequence: Syntheticoligonucleotide
  source 1..20: mol_type = other DNA
cacaaggttgacatcgtctg

SEQ ID NO: 344 - DNA, 20 bp, synthetic construct
  misc_feature 1..20: note = Description of Artificial Sequence: Syntheticoligonucleotide
  source 1..20: mol_type = other DNA
acacaaggttgacatcgtct

SEQ ID NO: 345 - DNA, 20 bp, synthetic construct
  misc_feature 1..20: note = Description of Artificial Sequence: Syntheticoligonucleotide
  source 1..20: mol_type = other DNA
cacacaaggttgacatcgtc

SEQ ID NO: 346 - DNA, 20 bp, synthetic construct
  misc_feature 1..20: note = Description of Artificial Sequence: Syntheticoligonucleotide
  source 1..20: mol_type = other DNA
tcacacaaggttgacatcgt

SEQ ID NO: 347 - DNA, 20 bp, synthetic construct
  misc_feature 1..20: note = Description of Artificial Sequence: Syntheticoligonucleotide
  source 1..20: mol_type = other DNA
ctcacacaaggttgacatcg

SEQ ID NO: 348 - DNA, 20 bp, synthetic construct
  misc_feature 1..20: note = Description of Artificial Sequence: Syntheticoligonucleotide
  source 1..20: mol_type = other DNA
actcacacaaggttgacatc

SEQ ID NO: 349 - DNA, 20 bp, synthetic construct
  misc_feature 1..20: note = Description of Artificial Sequence: Syntheticoligonucleotide
  source 1..20: mol_type = other DNA
cactcacacaaggttgacat

SEQ ID NO: 350 - DNA, 20 bp, synthetic construct
  misc_feature 1..20: note = Description of Artificial Sequence: Syntheticoligonucleotide
  source 1..20: mol_type = other DNA
acactcacacaaggttgaca

SEQ ID NO: 351 - DNA, 20 bp, synthetic construct
  misc_feature 1..20: note = Description of Artificial Sequence: Syntheticoligonucleotide
  source 1..20: mol_type = other DNA
cacactcacacaaggttgac

SEQ ID NO: 352 - DNA, 20 bp, synthetic construct
  misc_feature 1..20: note = Description of Artificial Sequence: Syntheticoligonucleotide
  source 1..20: mol_type = other DNA
tcacactcacacaaggttga

SEQ ID NO: 353 - DNA, 20 bp, synthetic construct
  misc_feature 1..20: note = Description of Artificial Sequence: Syntheticoligonucleotide
  source 1..20: mol_type = other DNA
gtcacactcacacaaggttg

SEQ ID NO: 354 - DNA, 20 bp, synthetic construct
  misc_feature 1..20: note = Description of Artificial Sequence: Syntheticoligonucleotide
  source 1..20: mol_type = other DNA
cgtcacactcacacaaggtt

SEQ ID NO: 355 - DNA, 20 bp, synthetic construct
  misc_feature 1..20: note = Description of Artificial Sequence: Syntheticoligonucleotide
  source 1..20: mol_type = other DNA
ccgtcacactcacacaaggt

SEQ ID NO: 356 - DNA, 20 bp, synthetic construct
  misc_feature 1..20: note = Description of Artificial Sequence: Syntheticoligonucleotide
  source 1..20: mol_type = other DNA
ccccgtcacactcacacaag

SEQ ID NO: 357 - DNA, 20 bp, synthetic construct
  misc_feature 1..20: note = Description of Artificial Sequence: Syntheticoligonucleotide
  source 1..20: mol_type = other DNA
cccttctcccacaggctgcc

SEQ ID NO: 358 - DNA, 20 bp, synthetic construct
  misc_feature 1..20: note = Description of Artificial Sequence: Syntheticoligonucleotide
  source 1..20: mol_type = other DNA
catcaaggtcagtcttttct

SEQ ID NO: 359 - DNA, 20 bp, synthetic construct
  misc_feature 1..20: note = Description of Artificial Sequence: Syntheticoligonucleotide
  source 1..20: mol_type = other DNA
ccaaccttcagaactcaata

SEQ ID NO: 360 - DNA, 20 bp, synthetic construct
  misc_feature 1..20: note = Description of Artificial Sequence: Syntheticoligonucleotide
  source 1..20: mol_type = other DNA
tccaaccttcagaactcaat

SEQ ID NO: 361 - DNA, 20 bp, synthetic construct
  misc_feature 1..20: note = Description of Artificial Sequence: Syntheticoligonucleotide
  source 1..20: mol_type = other DNA
ttccaaccttcagaactcaa

SEQ ID NO: 362 - DNA, 20 bp, synthetic construct
  misc_feature 1..20: note = Description of Artificial Sequence: Syntheticoligonucleotide
  source 1..20: mol_type = other DNA
gttccaaccttcagaactca

SEQ ID NO: 363 - DNA, 20 bp, synthetic construct
  misc_feature 1..20: note = Description of Artificial Sequence: Syntheticoligonucleotide
  source 1..20: mol_type = other DNA
agttccaaccttcagaactc

SEQ ID NO: 364 - DNA, 20 bp, synthetic construct
  misc_feature 1..20: note = Description of Artificial Sequence: Syntheticoligonucleotide
  source 1..20: mol_type = other DNA
cagttccaaccttcagaact

SEQ ID NO: 365 - DNA, 20 bp, synthetic construct
  misc_feature 1..20: note = Description of Artificial Sequence: Syntheticoligonucleotide
  source 1..20: mol_type = other DNA
gcagttccaaccttcagaac

SEQ ID NO: 366 - DNA, 20 bp, synthetic construct
  misc_feature 1..20: note = Description of Artificial Sequence: Syntheticoligonucleotide
  source 1..20: mol_type = other DNA
gtcccaggtctgcaaagtgg

SEQ ID NO: 367 - DNA, 20 bp, synthetic construct
  misc_feature 1..20: note = Description of Artificial Sequence: Syntheticoligonucleotide
  source 1..20: mol_type = other DNA
aagtcccaggtctgcaaagt

SEQ ID NO: 368 - DNA, 20 bp, synthetic construct
  misc_feature 1..20: note = Description of Artificial Sequence: Syntheticoligonucleotide
  source 1..20: mol_type = other DNA
aaagtcccaggtctgcaaag

SEQ ID NO: 369 - DNA, 20 bp, synthetic construct
  misc_feature 1..20: note = Description of Artificial Sequence: Syntheticoligonucleotide
  source 1..20: mol_type = other DNA
ggcacaagtccttacaaaga

SEQ ID NO: 370 - DNA, 20 bp, synthetic construct
  misc_feature 1..20: note = Description of Artificial Sequence: Syntheticoligonucleotide
  source 1..20: mol_type = other DNA
aggcacaagtccttacaaag

SEQ ID NO: 371 - DNA, 20 bp, synthetic construct
  misc_feature 1..20: note = Description of Artificial Sequence: Syntheticoligonucleotide
  source 1..20: mol_type = other DNA
tcaccctcagtatggagtag

SEQ ID NO: 372 - DNA, 20 bp, synthetic construct
  misc_feature 1..20: note = Description of Artificial Sequence: Syntheticoligonucleotide
  source 1..20: mol_type = other DNA
ttcaccctcagtatggagta

SEQ ID NO: 373 - DNA, 20 bp, synthetic construct
  misc_feature 1..20: note = Description of Artificial Sequence: Syntheticoligonucleotide
  source 1..20: mol_type = other DNA
tttcaccctcagtatggagt

SEQ ID NO: 374 - DNA, 20 bp, synthetic construct
  misc_feature 1..20: note = Description of Artificial Sequence: Syntheticoligonucleotide
  source 1..20: mol_type = other DNA
atttcaccctcagtatggag

SEQ ID NO: 375 - DNA, 20 bp, synthetic construct
  misc_feature 1..20: note = Description of Artificial Sequence: Syntheticoligonucleotide
  source 1..20: mol_type = other DNA
aatttcaccctcagtatgga

SEQ ID NO: 376 - DNA, 20 bp, synthetic construct
  misc_feature 1..20: note = Description of Artificial Sequence: Syntheticoligonucleotide
  source 1..20: mol_type = other DNA
ccttaatttcaccctcagta

SEQ ID NO: 377 - DNA, 20 bp, synthetic construct
  misc_feature 1..20: note = Description of Artificial Sequence: Syntheticoligonucleotide
  source 1..20: mol_type = other DNA
cccttaatttcaccctcagt

SEQ ID NO: 378 - DNA, 20 bp, synthetic construct
  misc_feature 1..20: note = Description of Artificial Sequence: Syntheticoligonucleotide
  source 1..20: mol_type = other DNA
tcccttaatttcaccctcag

SEQ ID NO: 379 - DNA, 20 bp, synthetic construct
  misc_feature 1..20: note = Description of Artificial Sequence: Syntheticoligonucleotide
  source 1..20: mol_type = other DNA
ttcccttaatttcaccctca

SEQ ID NO: 380 - DNA, 20 bp, synthetic construct
  misc_feature 1..20: note = Description of Artificial Sequence: Syntheticoligonucleotide
  source 1..20: mol_type = other DNA
cttcccttaatttcaccctc

SEQ ID NO: 381 - DNA, 20 bp, synthetic construct
  misc_feature 1..20: note = Description of Artificial Sequence: Syntheticoligonucleotide
  source 1..20: mol_type = other DNA
actcttgtgcctggactttg

SEQ ID NO: 382 - DNA, 20 bp, synthetic construct
  misc_feature 1..20: note = Description of Artificial Sequence: Syntheticoligonucleotide
  source 1..20: mol_type = other DNA
cactcttgtgcctggacttt

SEQ ID NO: 383 - DNA, 20 bp, synthetic construct
  misc_feature 1..20: note = Description of Artificial Sequence: Syntheticoligonucleotide
  source 1..20: mol_type = other DNA
ccactcttgtgcctggactt

SEQ ID NO: 384 - DNA, 20 bp, synthetic construct
  misc_feature 1..20: note = Description of Artificial Sequence: Syntheticoligonucleotide
  source 1..20: mol_type = other DNA
cccactcttgtgcctggact

SEQ ID NO: 385 - DNA, 20 bp, synthetic construct
  misc_feature 1..20: note = Description of Artificial Sequence: Syntheticoligonucleotide
  source 1..20: mol_type = other DNA
tcccactcttgtgcctggac

SEQ ID NO: 386 - DNA, 20 bp, synthetic construct
  misc_feature 1..20: note = Description of Artificial Sequence: Syntheticoligonucleotide
  source 1..20: mol_type = other DNA
gtcccactcttgtgcctgga

SEQ ID NO: 387 - DNA, 20 bp, synthetic construct
  misc_feature 1..20: note = Description of Artificial Sequence: Syntheticoligonucleotide
  source 1..20: mol_type = other DNA
ggtcccactcttgtgcctgg

SEQ ID NO: 388 - DNA, 20 bp, synthetic construct
  misc_feature 1..20: note = Description of Artificial Sequence: Syntheticoligonucleotide
  source 1..20: mol_type = other DNA
gggtcccactcttgtgcctg

SEQ ID NO: 389 - DNA, 20 bp, synthetic construct
  misc_feature 1..20: note = Description of Artificial Sequence: Syntheticoligonucleotide
  source 1..20: mol_type = other DNA
gtgccctggctcacatctgt

SEQ ID NO: 390 - DNA, 20 bp, synthetic construct
  misc_feature 1..20: note = Description of Artificial Sequence: Syntheticoligonucleotide
  source 1..20: mol_type = other DNA
agtgccctggctcacatctg

SEQ ID NO: 391 - DNA, 20 bp, synthetic construct
  misc_feature 1..20: note = Description of Artificial Sequence: Syntheticoligonucleotide
  source 1..20: mol_type = other DNA
cagtgccctggctcacatct

SEQ ID NO: 392 - DNA, 20 bp, synthetic construct
  misc_feature 1..20: note = Description of Artificial Sequence: Syntheticoligonucleotide
  source 1..20: mol_type = other DNA
gcagtgccctggctcacatc

SEQ ID NO: 393 - DNA, 20 bp, synthetic construct
  misc_feature 1..20: note = Description of Artificial Sequence: Syntheticoligonucleotide
  source 1..20: mol_type = other DNA
agcagtgccctggctcacat

SEQ ID NO: 394 - DNA, 20 bp, synthetic construct
  misc_feature 1..20: note = Description of Artificial Sequence: Syntheticoligonucleotide
  source 1..20: mol_type = other DNA
tgagcagtgccctggctcac

SEQ ID NO: 395 - DNA, 20 bp, synthetic construct
  misc_feature 1..20: note = Description of Artificial Sequence: Syntheticoligonucleotide
  source 1..20: mol_type = other DNA
gcatggcttccagctgggac

SEQ ID NO: 396 - DNA, 20 bp, synthetic construct
  misc_feature 1..20: note = Description of Artificial Sequence: Syntheticoligonucleotide
  source 1..20: mol_type = other DNA
agctgctccagcagaacaga

SEQ ID NO: 397 - DNA, 20 bp, synthetic construct
  misc_feature 1..20: note = Description of Artificial Sequence: Syntheticoligonucleotide
  source 1..20: mol_type = other DNA
tatatgttcagctgctccag

SEQ ID NO: 398 - DNA, 20 bp, synthetic construct
  misc_feature 1..20: note = Description of Artificial Sequence: Syntheticoligonucleotide
  source 1..20: mol_type = other DNA
gtatatgttcagctgctcca

SEQ ID NO: 399 - DNA, 20 bp, synthetic construct
  misc_feature 1..20: note = Description of Artificial Sequence: Syntheticoligonucleotide
  source 1..20: mol_type = other DNA
tgtatatgttcagctgctcc

SEQ ID NO: 400 - DNA, 20 bp, synthetic construct
  misc_feature 1..20: note = Description of Artificial Sequence: Syntheticoligonucleotide
  source 1..20: mol_type = other DNA
gcagggcaacatctatgtat

SEQ ID NO: 401 - DNA, 20 bp, synthetic construct
  misc_feature 1..20: note = Description of Artificial Sequence: Syntheticoligonucleotide
  source 1..20: mol_type = other DNA
ggcagggcaacatctatgta

SEQ ID NO: 402 - DNA, 20 bp, synthetic construct
  misc_feature 1..20: note = Description of Artificial Sequence: Syntheticoligonucleotide
  source 1..20: mol_type = other DNA
gggcagggcaacatctatgt

SEQ ID NO: 403 - DNA, 20 bp, synthetic construct
  misc_feature 1..20: note = Description of Artificial Sequence: Syntheticoligonucleotide
  source 1..20: mol_type = other DNA
tcactctggtgaatccaagc

SEQ ID NO: 404 - DNA, 20 bp, synthetic construct
  misc_feature 1..20: note = Description of Artificial Sequence: Syntheticoligonucleotide
  source 1..20: mol_type = other DNA
gtcactctggtgaatccaag

SEQ ID NO: 405 - DNA, 20 bp, synthetic construct
  misc_feature 1..20: note = Description of Artificial Sequence: Syntheticoligonucleotide
  source 1..20: mol_type = other DNA
agtcactctggtgaatccaa

SEQ ID NO: 406 - DNA, 20 bp, synthetic construct
  misc_feature 1..20: note = Description of Artificial Sequence: Syntheticoligonucleotide
  source 1..20: mol_type = other DNA
tagtcactctggtgaatcca

SEQ ID NO: 407 - DNA, 20 bp, synthetic construct
  misc_feature 1..20: note = Description of Artificial Sequence: Syntheticoligonucleotide
  source 1..20: mol_type = other DNA
atagtcactctggtgaatcc

SEQ ID NO: 408 - DNA, 20 bp, synthetic construct
  misc_feature 1..20: note = Description of Artificial Sequence: Syntheticoligonucleotide
  source 1..20: mol_type = other DNA
catagtcactctggtgaatc

SEQ ID NO: 409 - DNA, 20 bp, synthetic construct
  misc_feature 1..20: note = Description of Artificial Sequence: Syntheticoligonucleotide
  source 1..20: mol_type = other DNA
tcatagtcactctggtgaat

SEQ ID NO: 410 - DNA, 20 bp, synthetic construct
  misc_feature 1..20: note = Description of Artificial Sequence: Syntheticoligonucleotide
  source 1..20: mol_type = other DNA
ctggtctctgttgggtccca

SEQ ID NO: 411 - DNA, 20 bp, synthetic construct
  misc_feature 1..20: note = Description of Artificial Sequence: Syntheticoligonucleotide
  source 1..20: mol_type = other DNA
atcctgtgcttcaggccttc

SEQ ID NO: 412 - DNA, 20 bp, synthetic construct
  misc_feature 1..20: note = Description of Artificial Sequence: Syntheticoligonucleotide
  source 1..20: mol_type = other DNA
aatcctgtgcttcaggcctt

SEQ ID NO: 413 - DNA, 20 bp, synthetic construct
  misc_feature 1..20: note = Description of Artificial Sequence: Syntheticoligonucleotide
  source 1..20: mol_type = other DNA
ctaatcctgtgcttcaggcc

SEQ ID NO: 414 - DNA, 20 bp, synthetic construct
  misc_feature 1..20: note = Description of Artificial Sequence: Syntheticoligonucleotide
  source 1..20: mol_type = other DNA
cctaatcctgtgcttcaggc

SEQ ID NO: 415 - DNA, 20 bp, synthetic construct
  misc_feature 1..20: note = Description of Artificial Sequence: Syntheticoligonucleotide
  source 1..20: mol_type = other DNA
tcctaatcctgtgcttcagg

SEQ ID NO: 416 - DNA, 20 bp, synthetic construct
  misc_feature 1..20: note = Description of Artificial Sequence: Syntheticoligonucleotide
  source 1..20: mol_type = other DNA
gtcctaatcctgtgcttcag

SEQ ID NO: 417 - DNA, 20 bp, synthetic construct
  misc_feature 1..20: note = Description of Artificial Sequence: Syntheticoligonucleotide
  source 1..20: mol_type = other DNA
agtcctaatcctgtgcttca

SEQ ID NO: 418 - DNA, 20 bp, synthetic construct
  misc_feature 1..20: note = Description of Artificial Sequence: Syntheticoligonucleotide
  source 1..20: mol_type = other DNA
cagtcctaatcctgtgcttc

SEQ ID NO: 419 - DNA, 20 bp, synthetic construct
  misc_feature 1..20: note = Description of Artificial Sequence: Syntheticoligonucleotide
  source 1..20: mol_type = other DNA
tcagtcctaatcctgtgctt

SEQ ID NO: 420 - DNA, 20 bp, synthetic construct
  misc_feature 1..20: note = Description of Artificial Sequence: Syntheticoligonucleotide
  source 1..20: mol_type = other DNA
ttcagtcctaatcctgtgct

SEQ ID NO: 421 - DNA, 20 bp, synthetic construct
  misc_feature 1..20: note = Description of Artificial Sequence: Syntheticoligonucleotide
  source 1..20: mol_type = other DNA
cttcagtcctaatcctgtgc

SEQ ID NO: 422 - DNA, 20 bp, synthetic construct
  misc_feature 1..20: note = Description of Artificial Sequence: Syntheticoligonucleotide
  source 1..20: mol_type = other DNA
gcttcagtcctaatcctgtg

SEQ ID NO: 423 - DNA, 20 bp, synthetic construct
  misc_feature 1..20: note = Description of Artificial Sequence: Syntheticoligonucleotide
  source 1..20: mol_type = other DNA
ggagttgtaagcctcctttg

SEQ ID NO: 424 - DNA, 20 bp, synthetic construct
  misc_feature 1..20: note = Description of Artificial Sequence: Syntheticoligonucleotide
  source 1..20: mol_type = other DNA
gctctggtcaaggctttggg

SEQ ID NO: 425 - DNA, 20 bp, synthetic construct
  misc_feature 1..20: note = Description of Artificial Sequence: Syntheticoligonucleotide
  source 1..20: mol_type = other DNA
tgctctggtcaaggctttgg

SEQ ID NO: 426 - DNA, 20 bp, synthetic construct
  misc_feature 1..20: note = Description of Artificial Sequence: Syntheticoligonucleotide
  source 1..20: mol_type = other DNA
gtgctctggtcaaggctttg

SEQ ID NO: 427 - DNA, 20 bp, synthetic construct
  misc_feature 1..20: note = Description of Artificial Sequence: Syntheticoligonucleotide
  source 1..20: mol_type = other DNA
ggtgctctggtcaaggcttt

SEQ ID NO: 428 - DNA, 20 bp, synthetic construct
  misc_feature 1..20: note = Description of Artificial Sequence: Syntheticoligonucleotide
  source 1..20: mol_type = other DNA
tgaggtgctctggtcaaggc

SEQ ID NO: 429 - DNA, 20 bp, synthetic construct
  misc_feature 1..20: note = Description of Artificial Sequence: Syntheticoligonucleotide
  source 1..20: mol_type = other DNA
tttctcatggcagcagatgg

SEQ ID NO: 430 - DNA, 20 bp, synthetic construct
  misc_feature 1..20: note = Description of Artificial Sequence: Syntheticoligonucleotide
  source 1..20: mol_type = other DNA
tgctgagtttctttaggcag

SEQ ID NO: 431 - DNA, 20 bp, synthetic construct
  misc_feature 1..20: note = Description of Artificial Sequence: Syntheticoligonucleotide
  source 1..20: mol_type = other DNA
ctgctgagtttctttaggca

SEQ ID NO: 432 - DNA, 20 bp, synthetic construct
  misc_feature 1..20: note = Description of Artificial Sequence: Syntheticoligonucleotide
  source 1..20: mol_type = other DNA
gctgctgagtttctttaggc

SEQ ID NO: 433 - DNA, 20 bp, synthetic construct
  misc_feature 1..20: note = Description of Artificial Sequence: Syntheticoligonucleotide
  source 1..20: mol_type = other DNA
ggctgctgagtttctttagg

SEQ ID NO: 434 - DNA, 20 bp, synthetic construct
  misc_feature 1..20: note = Description of Artificial Sequence: Syntheticoligonucleotide
  source 1..20: mol_type = other DNA
aggctgctgagtttctttag

SEQ ID NO: 435 - DNA, 20 bp, synthetic construct
  misc_feature 1..20: note = Description of Artificial Sequence: Syntheticoligonucleotide
  source 1..20: mol_type = other DNA
gaggctgctgagtttcttta

SEQ ID NO: 436 - DNA, 20 bp, synthetic construct
  misc_feature 1..20: note = Description of Artificial Sequence: Syntheticoligonucleotide
  source 1..20: mol_type = other DNA
tgaggctgctgagtttcttt

SEQ ID NO: 437 - DNA, 20 bp, synthetic construct
  misc_feature 1..20: note = Description of Artificial Sequence: Syntheticoligonucleotide
  source 1..20: mol_type = other DNA
ctgccaagtccctcagggtt

SEQ ID NO: 438 - DNA, 20 bp, synthetic construct
  misc_feature 1..20: note = Description of Artificial Sequence: Syntheticoligonucleotide
  source 1..20: mol_type = other DNA
actgccaagtccctcagggt

SEQ ID NO: 439 - DNA, 20 bp, synthetic construct
  misc_feature 1..20: note = Description of Artificial Sequence: Syntheticoligonucleotide
  source 1..20: mol_type = other DNA
tactgccaagtccctcaggg

SEQ ID NO: 440 - DNA, 20 bp, synthetic construct
  misc_feature 1..20: note = Description of Artificial Sequence: Syntheticoligonucleotide
  source 1..20: mol_type = other DNA
ctactgccaagtccctcagg

SEQ ID NO: 441 - DNA, 20 bp, synthetic construct
  misc_feature 1..20: note = Description of Artificial Sequence: Syntheticoligonucleotide
  source 1..20: mol_type = other DNA
tctactgccaagtccctcag

SEQ ID NO: 442 - DNA, 20 bp, synthetic construct
  misc_feature 1..20: note = Description of Artificial Sequence: Syntheticoligonucleotide
  source 1..20: mol_type = other DNA
ttctactgccaagtccctca

SEQ ID NO: 443 - DNA, 20 bp, synthetic construct
  misc_feature 1..20: note = Description of Artificial Sequence: Syntheticoligonucleotide
  source 1..20: mol_type = other DNA
tttctactgccaagtccctc

SEQ ID NO: 444 - DNA, 20 bp, synthetic construct
  misc_feature 1..20: note = Description of Artificial Sequence: Syntheticoligonucleotide
  source 1..20: mol_type = other DNA
atttctactgccaagtccct

SEQ ID NO: 445 - DNA, 20 bp, synthetic construct
  misc_feature 1..20: note = Description of Artificial Sequence: Syntheticoligonucleotide
  source 1..20: mol_type = other DNA
gatttctactgccaagtccc

SEQ ID NO: 446 - DNA, 20 bp, synthetic construct
  misc_feature 1..20: note = Description of Artificial Sequence: Syntheticoligonucleotide
  source 1..20: mol_type = other DNA
ggatttctactgccaagtcc

SEQ ID NO: 447 - DNA, 20 bp, synthetic construct
  misc_feature 1..20: note = Description of Artificial Sequence: Syntheticoligonucleotide
  source 1..20: mol_type = other DNA
tggatttctactgccaagtc

SEQ ID NO: 448 - DNA, 20 bp, synthetic construct
  misc_feature 1..20: note = Description of Artificial Sequence: Syntheticoligonucleotide
  source 1..20: mol_type = other DNA
ctggatttctactgccaagt

SEQ ID NO: 449 - DNA, 20 bp, synthetic construct
  misc_feature 1..20: note = Description of Artificial Sequence: Syntheticoligonucleotide
  source 1..20: mol_type = other DNA
atcttaggctggccccaaga

SEQ ID NO: 450 - DNA, 20 bp, synthetic construct
  misc_feature 1..20: note = Description of Artificial Sequence: Syntheticoligonucleotide
  source 1..20: mol_type = other DNA
tgatcttaggctggccccaa

SEQ ID NO: 451 - DNA, 20 bp, synthetic construct
  misc_feature 1..20: note = Description of Artificial Sequence: Syntheticoligonucleotide
  source 1..20: mol_type = other DNA
tttatctgccagcactgatc

SEQ ID NO: 452 - DNA, 20 bp, synthetic construct
  misc_feature 1..20: note = Description of Artificial Sequence: Syntheticoligonucleotide
  source 1..20: mol_type = other DNA
atttatctgccagcactgat

SEQ ID NO: 453 - DNA, 20 bp, synthetic construct
  misc_feature 1..20: note = Description of Artificial Sequence: Syntheticoligonucleotide
  source 1..20: mol_type = other DNA
aatttatctgccagcactga

SEQ ID NO: 454 - DNA, 20 bp, synthetic construct
  misc_feature 1..20: note = Description of Artificial Sequence: Syntheticoligonucleotide
  source 1..20: mol_type = other DNA
tatatcctatctagcccacc

SEQ ID NO: 455 - DNA, 20 bp, synthetic construct
  misc_feature 1..20: note = Description of Artificial Sequence: Syntheticoligonucleotide
  source 1..20: mol_type = other DNA
gtatatcctatctagcccac

SEQ ID NO: 456 - DNA, 20 bp, synthetic construct
  misc_feature 1..20: note = Description of Artificial Sequence: Syntheticoligonucleotide
  source 1..20: mol_type = other DNA
agtatatcctatctagccca

SEQ ID NO: 457 - DNA, 20 bp, synthetic construct
  misc_feature 1..20: note = Description of Artificial Sequence: Syntheticoligonucleotide
  source 1..20: mol_type = other DNA
aaccccaagggcctctaact

SEQ ID NO: 458 - DNA, 20 bp, synthetic construct
  misc_feature 1..20: note = Description of Artificial Sequence: Syntheticoligonucleotide
  source 1..20: mol_type = other DNA
gcaaccagatgtccatattc

SEQ ID NO: 459 - DNA, 20 bp, synthetic construct
  misc_feature 1..20: note = Description of Artificial Sequence: Syntheticoligonucleotide
  source 1..20: mol_type = other DNA
ggcttaggacccctgaaaga

SEQ ID NO: 460 - DNA, 20 bp, synthetic construct
  misc_feature 1..20: note = Description of Artificial Sequence: Syntheticoligonucleotide
  source 1..20: mol_type = other DNA
ggcatgattgtgggcttagg

SEQ ID NO: 461 - DNA, 20 bp, synthetic construct
  misc_feature 1..20: note = Description of Artificial Sequence: Syntheticoligonucleotide
  source 1..20: mol_type = other DNA
aggcatgattgtgggcttag

SEQ ID NO: 462 - DNA, 20 bp, synthetic construct
  misc_feature 1..20: note = Description of Artificial Sequence: Syntheticoligonucleotide
  source 1..20: mol_type = other DNA
gtaacccttttcaaagctga

SEQ ID NO: 463 - DNA, 20 bp, synthetic construct
  misc_feature 1..20: note = Description of Artificial Sequence: Syntheticoligonucleotide
  source 1..20: mol_type = other DNA
ggtaacccttttcaaagctg

SEQ ID NO: 464 - DNA, 20 bp, synthetic construct
  misc_feature 1..20: note = Description of Artificial Sequence: Syntheticoligonucleotide
  source 1..20: mol_type = other DNA
gggtaacccttttcaaagct

SEQ ID NO: 465 - DNA, 20 bp, synthetic construct
  misc_feature 1..20: note = Description of Artificial Sequence: Syntheticoligonucleotide
  source 1..20: mol_type = other DNA
agggtaacccttttcaaagc

SEQ ID NO: 466 - DNA, 20 bp, synthetic construct
  misc_feature 1..20: note = Description of Artificial Sequence: Syntheticoligonucleotide
  source 1..20: mol_type = other DNA
cagggtaacccttttcaaag

SEQ ID NO: 467 - DNA, 20 bp, synthetic construct
  misc_feature 1..20: note = Description of Artificial Sequence: Syntheticoligonucleotide
  source 1..20: mol_type = other DNA
ccagggtaacccttttcaaa

SEQ ID NO: 468 - DNA, 20 bp, synthetic construct
  misc_feature 1..20: note = Description of Artificial Sequence: Syntheticoligonucleotide
  source 1..20: mol_type = other DNA
cccagggtaacccttttcaa

SEQ ID NO: 469 - DNA, 20 bp, synthetic construct
  misc_feature 1..20: note = Description of Artificial Sequence: Syntheticoligonucleotide
  source 1..20: mol_type = other DNA
gcccagggtaacccttttca

SEQ ID NO: 470 - DNA, 20 bp, synthetic construct
  misc_feature 1..20: note = Description of Artificial Sequence: Syntheticoligonucleotide
  source 1..20: mol_type = other DNA
tgctcaacatggcaaactca

SEQ ID NO: 471 - DNA, 20 bp, synthetic construct
  misc_feature 1..20: note = Description of Artificial Sequence: Syntheticoligonucleotide
  source 1..20: mol_type = other DNA
tcctgctcaacatggcaaac

SEQ ID NO: 472 - DNA, 20 bp, synthetic construct
  misc_feature 1..20: note = Description of Artificial Sequence: Syntheticoligonucleotide
  source 1..20: mol_type = other DNA
gtcctgctcaacatggcaaa

SEQ ID NO: 473 - DNA, 20 bp, synthetic construct
  misc_feature 1..20: note = Description of Artificial Sequence: Syntheticoligonucleotide
  source 1..20: mol_type = other DNA
cccgtacgtcccagcgtgat

SEQ ID NO: 474 - DNA, 20 bp, synthetic construct
  misc_feature 1..20: note = Description of Artificial Sequence: Syntheticoligonucleotide
  source 1..20: mol_type = other DNA
ggccagcgtccgtgtcaccc

SEQ ID NO: 475 - DNA, 17 bp, synthetic construct
  misc_feature 1..17: note = Description of Artificial Sequence: Syntheticoligonucleotide
  source 1..17: mol_type = other DNA
ccatgcgagctgataaa

SEQ ID NO: 476 - DNA, 17 bp, synthetic construct
  misc_feature 1..17: note = Description of Artificial Sequence: Syntheticoligonucleotide
  source 1..17: mol_type = other DNA
gcatcgtcagcttacct

SEQ ID NO: 477 - DNA, 17 bp, synthetic construct
  misc_feature 1..17: note = Description of Artificial Sequence: Syntheticoligonucleotide
  source 1..17: mol_type = other DNA
ctttgcttttactgacc

SEQ ID NO: 478 - DNA, 17 bp, synthetic construct
  misc_feature 1..17: note = Description of Artificial Sequence: Syntheticoligonucleotide
  source 1..17: mol_type = other DNA
tcaactggtttgtagac

SEQ ID NO: 479 - DNA, 20 bp, synthetic construct
  misc_feature 1..20: note = Description of Artificial Sequence: Syntheticoligonucleotide
  source 1..20: mol_type = other DNA
ccttgctcaggtcaactggt

SEQ ID NO: 480 - DNA, 20 bp, synthetic construct
  misc_feature 1..20: note = Description of Artificial Sequence: Syntheticoligonucleotide
  source 1..20: mol_type = other DNA
ggttgacatcgtctgcctgt

SEQ ID NO: 481 - DNA, 20 bp, synthetic construct
  misc_feature 1..20: note = Description of Artificial Sequence: Syntheticoligonucleotide
  source 1..20: mol_type = other DNA
gtcccactcttgtgcctgga

SEQ ID NO: 482 - DNA, 20 bp, synthetic construct
  misc_feature 1..20: note = Description of Artificial Sequence: Syntheticoligonucleotide
  source 1..20: mol_type = other DNA
gacatcgtctgcctgtggct

SEQ ID NO: 483 - DNA, 20 bp, synthetic construct
  misc_feature 1..20: note = Description of Artificial Sequence: Syntheticoligonucleotide
  source 1..20: mol_type = other DNA
cccactcttgtgcctggact

SEQ ID NO: 484 - DNA, 20 bp, synthetic construct
  misc_feature 1..20: note = Description of Artificial Sequence: Syntheticoligonucleotide
  source 1..20: mol_type = other DNA
gtcccaggtctgcaaagtgg

SEQ ID NO: 485 - DNA, 20 bp, synthetic construct
  misc_feature 1..20: note = Description of Artificial Sequence: Syntheticoligonucleotide
  source 1..20: mol_type = other DNA
gtctgcctgtggctccacga

SEQ ID NO: 486 - DNA, 20 bp, synthetic construct
  misc_feature 1..20: note = Description of Artificial Sequence: Syntheticoligonucleotide
  source 1..20: mol_type = other DNA
agtcactctggtgaatccaa

SEQ ID NO: 487 - RNA, 20 nt, synthetic construct
  misc_feature 1..20: note = Description of Artificial Sequence: Syntheticoligonucleotide
  source 1..20: mol_type = other RNA
cggaaggtcagcttgtgggt

SEQ ID NO: 488 - RNA, 30 nt, synthetic construct
  misc_feature 1..30: note = Description of Artificial Sequence: Syntheticoligonucleotide
  source 1..30: mol_type = other RNA
gtgtctccaatgcctgcttcttcagctggt

SEQ ID NO: 489 - RNA, 20 nt, synthetic construct
  misc_feature 1..20: note = Description of Artificial Sequence: Syntheticoligonucleotide
  source 1..20: mol_type = other RNA
tcaactggtttgtagactat

SEQ ID NO: 490 - RNA, 20 nt, synthetic construct
  misc_feature 1..20: note = Description of Artificial Sequence: Syntheticoligonucleotide
  source 1..20: mol_type = other RNA
tatgatggatgttgcctaat

SEQ ID NO: 491 - RNA, 20 nt, synthetic construct
  misc_feature 1..20: note = Description of Artificial Sequence: Syntheticoligonucleotide
  source 1..20: mol_type = other RNA
gggctcagccatcctggttc

SEQ ID NO: 492 - RNA, 18 nt, synthetic construct
  misc_feature 1..18: note = Description of Artificial Sequence: Syntheticoligonucleotide
  source 1..18: mol_type = other RNA
tttcctccgccagggacg

SEQ ID NO: 493 - RNA, 20 nt, synthetic construct
  misc_feature 1..20: note = Description of Artificial Sequence: Syntheticoligonucleotide
  source 1..20: mol_type = other RNA
gtctttgcttttactgacca

SEQ ID NO: 494 - RNA, 20 nt, synthetic construct
  misc_feature 1..20: note = Description of Artificial Sequence: Syntheticoligonucleotide
  source 1..20: mol_type = other RNA
ttttttgtcatcgcttccag

SEQ ID NO: 495 - RNA, 20 nt, synthetic construct
  misc_feature 1..20: note = Description of Artificial Sequence: Syntheticoligonucleotide
  source 1..20: mol_type = other RNA
accagttgacctgagcaagg

SEQ ID NO: 496 - RNA, 20 nt, synthetic construct
  misc_feature 1..20: note = Description of Artificial Sequence: Syntheticoligonucleotide
  source 1..20: mol_type = other RNA
acaggcagacgatgtcaacc

SEQ ID NO: 497 - RNA, 20 nt, synthetic construct
  misc_feature 1..20: note = Description of Artificial Sequence: Syntheticoligonucleotide
  source 1..20: mol_type = other RNA
tccaggcacaagagtgggac

SEQ ID NO: 498 - RNA, 20 nt, synthetic construct
  misc_feature 1..20: note = Description of Artificial Sequence: Syntheticoligonucleotide
  source 1..20: mol_type = other RNA
agccacaggcagacgatgtc

SEQ ID NO: 499 - RNA, 20 nt, synthetic construct
  misc_feature 1..20: note = Description of Artificial Sequence: Syntheticoligonucleotide
  source 1..20: mol_type = other RNA
agtccaggcacaagagtggg

SEQ ID NO: 500 - RNA, 20 nt, synthetic construct
  misc_feature 1..20: note = Description of Artificial Sequence: Syntheticoligonucleotide
  source 1..20: mol_type = other RNA
ccactttgcagacctgggac

SEQ ID NO: 501 - RNA, 20 nt, synthetic construct
  misc_feature 1..20: note = Description of Artificial Sequence: Syntheticoligonucleotide
  source 1..20: mol_type = other RNA
tcgtggagccacaggcagac

SEQ ID NO: 502 - RNA, 20 nt, synthetic construct
  misc_feature 1..20: note = Description of Artificial Sequence: Syntheticoligonucleotide
  source 1..20: mol_type = other RNA
ttggattcaccagagtgact

SEQ ID NO: 503 - RNA, 25 nt, synthetic construct
  misc_feature 1..25: note = Description of Artificial Sequence: Syntheticoligonucleotide
  source 1..25: mol_type = other RNA
gtccactaacctttcaggccagcgt

SEQ ID NO: 504 - RNA, 25 nt, synthetic construct
  misc_feature 1..25: note = Description of Artificial Sequence: Syntheticoligonucleotide
  source 1..25: mol_type = other RNA
gcatcgtcagcttaccttggctttt

SEQ ID NO: 505 - RNA, 25 nt, synthetic construct
  misc_feature 1..25: note = Description of Artificial Sequence: Syntheticoligonucleotide
  source 1..25: mol_type = other RNA
ccatgcgagctgataaaatataaaa

SEQ ID NO: 506 - RNA, 25 nt, synthetic construct
  misc_feature 1..25: note = Description of Artificial Sequence: Syntheticoligonucleotide
  source 1..25: mol_type = other RNA
gactatttgcacctggagatgagag